SEQUENCE LISTING (18 sequences)

SEQ ID NO: 1 - DNA, 133 bp, adeno-associated virus 2
tagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 199 bp, Homo sapiens
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggg

SEQ ID NO: 3 - DNA, 806 bp, Homo sapiens
cagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcc

SEQ ID NO: 4 - DNA, 434 bp, Artificial Sequence
  Chicken beta-actin promoter exon-intron-exon (Ex/In/Ex)
tcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgatatcgaattcctgcag

SEQ ID NO: 5 - DNA, 2580 bp, Homo sapiens
atgaggcgctgccggtgggccgccctggccctggggctgctgcgcctctgcttggctcaggccaacttcgccccgcacttcttcgacaacggggtcggcagcaccaacggaaacatggctctgttcagcctcccagaggacacccctgtaggctctcacgtatacaccctgaatgggacagaccctgagggagaccccatctcctaccacatcagctttgaccccagcactagaagcgtcttttctgttgaccccacttttggaaacatcaccctggttgaagagctggacagagagagggaagatgagattgaagccatcatcagcatttctgatggcctgaatctggtggccgaaaaagtcgtgatcctggtgaccgatgccaatgatgaggcgcccaggttcatccaggagccttatgttgccctggttcccgaggacatacctgctgggagcatcatctttaaggtccatgcagtggacagggacacaggctctggagggagtgtcacctacttcctgcagaacctgcactccccatttgccgtggaccgccacagcggtgtgctgcgcctccaggctggggccactctggactacgagaggtcccggacccactacatcaccgtggtcgccaaggatggcggtgggaggcttcatggggctgatgtggtgttctcagccaccaccacggtcacggtcaatgtggaggatgttcaggacatggcccctgtcttcgtgggcacaccctactatggctatgtgtacgaggacacccttccgggctcggaggtactgaaggtggtcgccatggatggagaccggggcaaacccaatcgaattctctacagccttgtaaatgggaacgatggagcctttgaaattaatgagacatctggagccatctccatcactcagagcccggcccagctccagagagaggtgtatgagctgcatgtacaggtgactgaaatgagccctgcggggagcccagctgcccaggccaccgtcccagtcaccatcaggattgtggacctcaacaaccacccgccaacattctatggagagagcggaccccaaaacaggtttgagctgtccatgaatgagcacccaccccagggagagatcctgcggggcctcaagatcaccgtcaatgactccgaccagggagccaatgccaaattcaacttgcagctggtgggacccaggggcatcttccgagtggttccacagacagtcctgaatgaagcccaagtcacaatcattgtggagaactcagctgccattgactttgaaaagtccaaagtattaaccttcaagctcctggctgttgaagtgaacaccccagagaagttcagttccacagcggatgttgtgatccagctcctggacaccaatgacaatgtccccaagttcgactccctctactacgttgccaggattcctgagaacgccccagggggctccagcgtggtggctgtcacagctgtggatccagatacaggaccctggggcgaagtgaaatattccacctatgggactggggcagacctcttcctgatccacccatccactgggcttatctacacccagccctgggctagcctggacgctgaggccactgccaggtacaacttctatgtgaaggcagaggacatggaaggcaagtacagcgtagctgaggtgtttatcacactgctggatgtcaatgaccacccccctcagtttggaaagagcgttcagaagaagacgatggtgctagggaccccagtgaaaattgaggccatagacgaggatgcagaggaacccaacaacctggtggactattccatcacccatgcagagcccgccaacgtgttcgacatcaattcccacacgggggagatctggctcaagaattccatccgctccctggatgccctgcacaacatcacacctggaagggactgcctatggtccctagaggtgcaggccaaggaccggggctccccatccttcagcaccacagccttactcaagattgacatcacagatgctgagaccctctcccggagccccatggctgccttcctgatacagaccaaggacaaccccatgaaggccgtgggtgtgctggccggcaccatggccaccgtcgtggccatcactgtcctcatctccaccgccaccttctggcgcaacaagaagtctaacaaggtcctgccaatgcggcgggtgctccgcaagcggcccagccctgcgccccgcaccatccgcattgagtggctcaagtccaagagcaccaaagccgctaccaagttcatgctcaaagagaaacctcccaatgagaactgtaacaacaacagcccagaaagctctctgctcccgagagctccggctctccctccaccacccagcgtggcgcccagcactggcgcagcccagtggaccgtgcctactgtctctggctctctcactccgcagccgacccaacccccgccaaaacccaaaactatgggaagccccgtccagtcaactctgatctctgagctcaagcaaaagtttgagaagaagagtgtgcacaacaaggcttacttctaa

SEQ ID NO: 6 - protein, 859 aa, Homo sapiens
MRRCRWAALALGLLRLCLAQANFAPHFFDNGVGSTNGNMALFSLPEDTPVGSHVYTLNGTDPEGDPISYHISFDPSTRSVFSVDPTFGNITLVEELDREREDEIEAIISISDGLNLVAEKVVILVTDANDEAPRFIQEPYVALVPEDIPAGSIIFKVHAVDRDTGSGGSVTYFLQNLHSPFAVDRHSGVLRLQAGATLDYERSRTHYITVVAKDGGGRLHGADVVFSATTTVTVNVEDVQDMAPVFVGTPYYGYVYEDTLPGSEVLKVVAMDGDRGKPNRILYSLVNGNDGAFEINETSGAISITQSPAQLQREVYELHVQVTEMSPAGSPAAQATVPVTIRIVDLNNHPPTFYGESGPQNRFELSMNEHPPQGEILRGLKITVNDSDQGANAKFNLQLVGPRGIFRVVPQTVLNEAQVTIIVENSAAIDFEKSKVLTFKLLAVEVNTPEKFSSTADVVIQLLDTNDNVPKFDSLYYVARIPENAPGGSSVVAVTAVDPDTGPWGEVKYSTYGTGADLFLIHPSTGLIYTQPWASLDAEATARYNFYVKAEDMEGKYSVAEVFITLLDVNDHPPQFGKSVQKKTMVLGTPVKIEAIDEDAEEPNNLVDYSITHAEPANVFDINSHTGEIWLKNSIRSLDALHNITPGRDCLWSLEVQAKDRGSPSFSTTALLKIDITDAETLSRSPMAAFLIQTKDNPMKAVGVLAGTMATVVAITVLISTATFWRNKKSNKVLPMRRVLRKRPSPAPRTIRIEWLKSKSTKAATKFMLKEKPPNENCNNNSPESSLLPRAPALPPPPSVAPSTGAAQWTVPTVSGSLTPQPTQPPPKPKTMGSPVQSTLISELKQKFEKKSVHNKAYF

SEQ ID NO: 7 - DNA, 589 bp, Artificial Sequence
  WPRE
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 8 - DNA, 228 bp, Artificial Sequence
  Bovine growth hormone polyadenylation tail
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 9 - DNA, 118 bp, adeno-associated virus 2
tgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 10 - DNA, 3432 bp, Artificial Sequence
  Vector GRK1.CDHR1.pA (5′ITR to 3′ITR, inclusive)
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggggttaacgccaccatgaggcgctgccggtgggccgccctggccctggggctgctgcgcctctgcttggctcaggccaacttcgccccgcacttcttcgacaacggggtcggcagcaccaacggaaacatggctctgttcagcctcccagaggacacccctgtaggctctcacgtatacaccctgaatgggacagaccctgagggagaccccatctcctaccacatcagctttgaccccagcactagaagcgtcttttctgttgaccccacttttggaaacatcaccctggttgaagagctggacagagagagggaagatgagattgaagccatcatcagcatttctgatggcctgaatctggtggccgaaaaagtcgtgatcctggtgaccgatgccaatgatgaggcgcccaggttcatccaggagccttatgttgccctggttcccgaggacatacctgctgggagcatcatctttaaggtccatgcagtggacagggacacaggctctggagggagtgtcacctacttcctgcagaacctgcactccccatttgccgtggaccgccacagcggtgtgctgcgcctccaggctggggccactctggactacgagaggtcccggacccactacatcaccgtggtcgccaaggatggcggtgggaggcttcatggggctgatgtggtgttctcagccaccaccacggtcacggtcaatgtggaggatgttcaggacatggcccctgtcttcgtgggcacaccctactatggctatgtgtacgaggacacccttccgggctcggaggtactgaaggtggtcgccatggatggagaccggggcaaacccaatcgaattctctacagccttgtaaatgggaacgatggagcctttgaaattaatgagacatctggagccatctccatcactcagagcccggcccagctccagagagaggtgtatgagctgcatgtacaggtgactgaaatgagccctgcggggagcccagctgcccaggccaccgtcccagtcaccatcaggattgtggacctcaacaaccacccgccaacattctatggagagagcggaccccaaaacaggtttgagctgtccatgaatgagcacccaccccagggagagatcctgcggggcctcaagatcaccgtcaatgactccgaccagggagccaatgccaaattcaacttgcagctggtgggacccaggggcatcttccgagtggttccacagacagtcctgaatgaagcccaagtcacaatcattgtggagaactcagctgccattgactttgaaaagtccaaagtattaaccttcaagctcctggctgttgaagtgaacaccccagagaagttcagttccacagcggatgttgtgatccagctcctggacaccaatgacaatgtccccaagttcgactccctctactacgttgccaggattcctgagaacgccccagggggctccagcgtggtggctgtcacagctgtggatccagatacaggaccctggggcgaagtgaaatattccacctatgggactggggcagacctcttcctgatccacccatccactgggcttatctacacccagccctgggctagcctggacgctgaggccactgccaggtacaacttctatgtgaaggcagaggacatggaaggcaagtacagcgtagctgaggtgtttatcacactgctggatgtcaatgaccacccccctcagtttggaaagagcgttcagaagaagacgatggtgctagggaccccagtgaaaattgaggccatagacgaggatgcagaggaacccaacaacctggtggactattccatcacccatgcagagcccgccaacgtgttcgacatcaattcccacacgggggagatctggctcaagaattccatccgctccctggatgccctgcacaacatcacacctggaagggactgcctatggtccctagaggtgcaggccaaggaccggggctccccatccttcagcaccacagccttactcaagattgacatcacagatgctgagaccctctcccggagccccatggctgccttcctgatacagaccaaggacaaccccatgaaggccgtgggtgtgctggccggcaccatggccaccgtcgtggccatcactgtcctcatctccaccgccaccttctggcgcaacaagaagtctaacaaggtcctgccaatgcggcgggtgctccgcaagcggcccagccctgcgccccgcaccatccgcattgagtggctcaagtccaagagcaccaaagccgctaccaagttcatgctcaaagagaaacctcccaatgagaactgtaacaacaacagcccagaaagctctctgctcccgagagctccggctctccctccaccacccagcgtggcgcccagcactggcgcagcccagtggaccgtgcctactgtctctggctctctcactccgcagccgacccaacccccgccaaaacccaaaactatgggaagccccgtccagtcaactctgatctctgagctcaagcaaaagtttgagaagaagagtgtgcacaacaaggcttacttctaaaagctctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgca

SEQ ID NO: 11 - DNA, 4056 bp, Artificial Sequence
  Vector GRK1.CDHR1.WPRE.pA (5′ITR to 3′ITR, inclusive)
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggggttattgttaacgccaccatgaggcgctgccggtgggccgccctggccctggggctgctgcgcctctgcttggctcaggccaacttcgccccgcacttcttcgacaacggggtcggcagcaccaacggaaacatggctctgttcagcctcccagaggacacccctgtaggctctcacgtatacaccctgaatgggacagaccctgagggagaccccatctcctaccacatcagctttgaccccagcactagaagcgtcttttctgttgaccccacttttggaaacatcaccctggttgaagagctggacagagagagggaagatgagattgaagccatcatcagcatttctgatggcctgaatctggtggccgaaaaagtcgtgatcctggtgaccgatgccaatgatgaggcgcccaggttcatccaggagccttatgttgccctggttcccgaggacatacctgctgggagcatcatctttaaggtccatgcagtggacagggacacaggctctggagggagtgtcacctacttcctgcagaacctgcactccccatttgccgtggaccgccacagcggtgtgctgcgcctccaggctggggccactctggactacgagaggtcccggacccactacatcaccgtggtcgccaaggatggcggtgggaggcttcatggggctgatgtggtgttctcagccaccaccacggtcacggtcaatgtggaggatgttcaggacatggcccctgtcttcgtgggcacaccctactatggctatgtgtacgaggacacccttccgggctcggaggtactgaaggtggtcgccatggatggagaccggggcaaacccaatcgaattctctacagccttgtaaatgggaacgatggagcctttgaaattaatgagacatctggagccatctccatcactcagagcccggcccagctccagagagaggtgtatgagctgcatgtacaggtgactgaaatgagccctgcggggagcccagctgcccaggccaccgtcccagtcaccatcaggattgtggacctcaacaaccacccgccaacattctatggagagagcggaccccaaaacaggtttgagctgtccatgaatgagcacccaccccagggagagatcctgcggggcctcaagatcaccgtcaatgactccgaccagggagccaatgccaaattcaacttgcagctggtgggacccaggggcatcttccgagtggttccacagacagtcctgaatgaagcccaagtcacaatcattgtggagaactcagctgccattgactttgaaaagtccaaagtattaaccttcaagctcctggctgttgaagtgaacaccccagagaagttcagttccacagcggatgttgtgatccagctcctggacaccaatgacaatgtccccaagttcgactccctctactacgttgccaggattcctgagaacgccccagggggctccagcgtggtggctgtcacagctgtggatccagatacaggaccctggggcgaagtgaaatattccacctatgggactggggcagacctcttcctgatccacccatccactgggcttatctacacccagccctgggctagcctggacgctgaggccactgccaggtacaacttctatgtgaaggcagaggacatggaaggcaagtacagcgtagctgaggtgtttatcacactgctggatgtcaatgaccacccccctcagtttggaaagagcgttcagaagaagacgatggtgctagggaccccagtgaaaattgaggccatagacgaggatgcagaggaacccaacaacctggtggactattccatcacccatgcagagcccgccaacgtgttcgacatcaattcccacacgggggagatctggctcaagaattccatccgctccctggatgccctgcacaacatcacacctggaagggactgcctatggtccctagaggtgcaggccaaggaccggggctccccatccttcagcaccacagccttactcaagattgacatcacagatgctgagaccctctcccggagccccatggctgccttcctgatacagaccaaggacaaccccatgaaggccgtgggtgtgctggccggcaccatggccaccgtcgtggccatcactgtcctcatctccaccgccaccttctggcgcaacaagaagtctaacaaggtcctgccaatgcggcgggtgctccgcaagcggcccagccctgcgccccgcaccatccgcattgagtggctcaagtccaagagcaccaaagccgctaccaagttcatgctcaaagagaaacctcccaatgagaactgtaacaacaacagcccagaaagctctctgctcccgagagctccggctctccctccaccacccagcgtggcgcccagcactggcgcagcccagtggaccgtgcctactgtctctggctctctcactccgcagccgacccaacccccgccaaaacccaaaactatgggaagccccgtccagtcaactctgatctctgagctcaagcaaaagtttgagaagaagagtgtgcacaacaaggcttacttctaaaagcttgggagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgca

SEQ ID NO: 12 - DNA, 4491 bp, Artificial Sequence
  Vector GRK1.In.CDHR1.WPRE.pA (5′ITR to 3′ITR, inclusive)
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggggttattgtttcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcctagcttgatatcgaattcctgcagcaacgccaccaugaggcgcugccggugggccgcccuggcccuggggcugcugcgccucugcuuggcucaggccaacuucgccccgcacuucuucgacaacggggucggcagcaccaacggaaacauggcucuguucagccucccagaggacaccccuguaggcucucacguauacacccugaaugggacagacccugagggagaccccaucuccuaccacaucagcuuugaccccagcacuagaagcgucuuuucuguugaccccacuuuuggaaacaucacccugguugaagagcuggacagagagagggaagaugagauugaagccaucaucagcauuucugauggccugaaucugguggccgaaaaagucgugauccuggugaccgaugccaaugaugaggcgcccagguucauccaggagccuuauguugcccugguucccgaggacauaccugcugggagcaucaucuuuaagguccaugcaguggacagggacacaggcucuggagggagugucaccuacuuccugcagaaccugcacuccccauuugccguggaccgccacagcggugugcugcgccuccaggcuggggccacucuggacuacgagaggucccggacccacuacaucaccguggucgccaaggauggcggugggaggcuucauggggcugaugugguguucucagccaccaccacggucacggucaauguggaggauguucaggacauggccccugucuucgugggcacacccuacuauggcuauguguacgaggacacccuuccgggcucggagguacugaagguggucgccauggauggagaccggggcaaacccaaucgaauucucuacagccuuguaaaugggaacgauggagccuuugaaauuaaugagacaucuggagccaucuccaucacucagagcccggcccagcuccagagagagguguaugagcugcauguacaggugacugaaaugagcccugcggggagcccagcugcccaggccaccgucccagucaccaucaggauuguggaccucaacaaccacccgccaacauucuauggagagagcggaccccaaaacagguuugagcuguccaugaaugagcacccaccccagggagagauccugcggggccucaagaucaccgucaaugacuccgaccagggagccaaugccaaauucaacuugcagcuggugggacccaggggcaucuuccgagugguuccacagacaguccugaaugaagcccaagucacaaucauuguggagaacucagcugccauugacuuugaaaaguccaaaguauuaaccuucaagcuccuggcuguugaagugaacaccccagagaaguucaguuccacagcggauguugugauccagcuccuggacaccaaugacaauguccccaaguucgacucccucuacuacguugccaggauuccugagaacgccccagggggcuccagcgugguggcugucacagcuguggauccagauacaggacccuggggcgaagugaaauauuccaccuaugggacuggggcagaccucuuccugauccacccauccacugggcuuaucuacacccagcccugggcuagccuggacgcugaggccacugccagguacaacuucuaugugaaggcagaggacauggaaggcaaguacagcguagcugagguguuuaucacacugcuggaugucaaugaccaccccccucaguuuggaaagagcguucagaagaagacgauggugcuagggaccccagugaaaauugaggccauagacgaggaugcagaggaacccaacaaccugguggacuauuccaucacccaugcagagcccgccaacguguucgacaucaauucccacacgggggagaucuggcucaagaauuccauccgcucccuggaugcccugcacaacaucacaccuggaagggacugccuauggucccuagaggugcaggccaaggaccggggcuccccauccuucagcaccacagccuuacucaagauugacaucacagaugcugagacccucucccggagccccauggcugccuuccugauacagaccaaggacaaccccaugaaggccgugggugugcuggccggcaccauggccaccgucguggccaucacuguccucaucuccaccgccaccuucuggcgcaacaagaagucuaacaagguccugccaaugcggcgggugcuccgcaagcggcccagcccugcgccccgcaccauccgcauugaguggcucaaguccaagagcaccaaagccgcuaccaaguucaugcucaaagagaaaccucccaaugagaacuguaacaacaacagcccagaaagcucucugcucccgagagcuccggcucucccuccaccacccagcguggcgcccagcacuggcgcagcccaguggaccgugccuacugucucuggcucucucacuccgcagccgacccaacccccgccaaaacccaaaacuaugggaagccccguccagucaacucugaucucugagcucaagcaaaaguuugagaagaagagugugcacaacaaggcuuacuucuagaagcttgggagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgca

SEQ ID NO: 13 - protein, 89 aa, Homo sapiens
FSLPEDTPVGSHVYTLNGTDPEGDPISYHISFDPSTRSVFSVDPTFGNITLVEELDREREDEIEAIISISDGLNLVAEKVVILVTDAND

SEQ ID NO: 14 - protein, 103 aa, Homo sapiens
PYVALVPEDIPAGSIIFKVHAVDRDTGSGGSVTYFLQNLHSPFAVDRHSGVLRLQAGATLDYERSRTHYITVVAKDGGGRLHGADVVFSATTTVTVNVEDVQD

SEQ ID NO: 15 - protein, 100 aa, Homo sapiens
PYYGYVYEDTLPGSEVLKVVAMDGDRGKPNRILYSLVNGNDGAFEINETSGAISITQSPAQLQREVYELHVQVTEMSPAGSPAAQATVPVTIRIVDLNNH

SEQ ID NO: 16 - protein, 106 aa, Homo sapiens
FELSMNEHPPQGEILRGLKITVNDSDQGANAKFNLQLVGPRGIFRVVPQTVLNEAQVTIIVENSAAIDFEKSKVLTFKLLAVEVNTPEKFSSTADVVIQLLDTNDN

SEQ ID NO: 17 - protein, 96 aa, Homo sapiens
YVARIPENAPGGSSVVAVTAVDPDTGPWGEVKYSTYGTGADLFLIHPSTGLIYTQPWASLDAEATARYNFYVKAEDMEGKYSVAEVFITLLDVNDH

SEQ ID NO: 18 - protein, 92 aa, Homo sapiens
GTPVKIEAIDEDAEEPNNLVDYSITHAEPANVFDINSHTGEIWLKNSIRSLDALHNITPGRDCLWSLEVQAKDRGSPSFSTTALLKIDITDA

(19) United States
(12) Patent Application Publication
(10) Pub. No.: US 2024/0191257 A1
(43) Pub. Date: Jun. 13, 2024

(54) Gene Therapy for Retinal Disease

(71) Applicant: OXFORD UNIVERSITY INNOVATION LIMITED, Oxford, Oxfordshire (GB)

(72) Inventors: Imran H. Yusuf, Oxford, Oxfordshire (GB); Robert E. MacLaren, Oxford, Oxfordshire (GB); Peter Charbel Issa, Oxford, Oxfordshire (GB); Michelle E. McClements, Oxford, Oxfordshire (GB)

(21) Appl. No.: 18/553,295

(22) PCT Filed: Mar. 30, 2022
(86) PCT No.: PCT/GB2022/050800

§ 371 Date: Sep. 29, 2023

(30) Foreign Application Priority Data
Mar. 31, 2021 (GB) 2104611.5

(51) Int. Cl. C12N 15/86; A61K 48/00; A61P 27/02; C07K 14/705

(52) U.S. Cl. CPC C12N 15/86; A61K 48/0058; A61K 48/0075; A61P 27/02; C07K 14/705; C12N 2750/14143; C12N 2830/008; C12N 2830/48; C12N 2830/50

(57) ABSTRACT

The invention provides methods for treating, preventing or reversing retinal degeneration. The methods administering to the subject a vector that expresses a Cadherin-related family member 1 (CDHR1) polypeptide. The invention also provides gene therapy vectors that expresses a CDHR1 polypeptide, host cells that express the gene therapy vectors, and pharmaceutical compositions comprising the gene therapy vector.

### FIELD

The invention relates to gene therapy vectors for treating, preventing or reversing retinal degeneration.

### BACKGROUND

CDHR1 is essential for outer segment disc morphogenesis in the mammalian retina. CDHR1-associated retinal degeneration is a recessively inherited human disease for which there is no effective treatment. Biallelic null variants in CDHR1 result in mis-stacking of the outer segment discs of rod and cone photoreceptors, with early rod and cone dysfunction associated with shortened photoreceptor outer segments and progressive photoreceptor cell death. This phenotype is recapitulated by the Cdhr1^{-/-} murine model. Progressive photoreceptor cell death results in legal blindness in all affected patients before the age of 60. Patients with CDHR1-associated retinal degeneration experience both rod and cone photoreceptor degeneration, which means that they lose both their peripheral vision/visual field and also their central reading/colour vision which is normally preserved until late in many other forms of retinitis pigmentosa. This makes CDHR1-associated retinal degeneration visually debilitating. CDHR1-associated retinal degeneration is also relatively common: the global genetic prevalence is estimated at ˜211,832 affected individuals worldwide, including ˜62,665 individuals in Europe and North America. Biallelic hypomorphic variants in CDHR1 have also recently been identified as one of the main genetic causes of macular dystrophy.

CDHR1 encodes a photoreceptor-specific cadherin. Cadherins form a superfamily of proteins characterised by the presence of two or more extracellular cadherin repeats and serve crucial roles in cell-to-cell adhesion. In highly evolved sensory cells, such as the photoreceptors in the retina and the hair cells of the inner ear, cadherins serve more complex functions, principally through heterophilic interactions with other proteins to support the function of the cilia and outer segment. The cilia is crucial in that that links photoreceptor inner segments, where proteins are manufactured, to the outer segment discs which are highly specialised structures that support the process of phototransduction (i.e. conversion of light energy into electrical potentials). The heterophilic binding partner of CDHR1 in the inner segment is unknown.

Methods are being developed to deliver gene therapy to the cells of the retina using adeno-associated virus (AAV) (MacLaren et al. (2016), American Academy of Ophthalmology 123(10) Suppl. S98-S106; Fischer M D et al. (2017), Molecular Therapy. 25 (8): 1854-1865). However, membrane-bound proteins, such as cadherins, are not usually considered attractive targets for gene therapy as it is presumed they are more likely to evoke undesirable immune responses. To date no transgene expressing a cadherin has been shown to ameliorate any disease phenotype in vivo in any organ.

### SUMMARY

The inventors have undertaken longitudinal deep phenotyping of the Cdhr1^{-/-} mouse model to demonstrate the extent of mimicry of the human phenotype and identify suitable outcome measures to assess downstream gene rescue. They have designed vector constructs that efficiently express CDHR1 protein in the photoreceptor cells of the Cdhr1^{-/-} mouse retina. Treatment results in significant and unexpected long-term improvement of retinal function in both rod- and cone-specific responses. Surprisingly, rod and cone rescue was achieved through the prevention of photoreceptor cell death and, even more unexpected, regeneration of photoreceptor outer segments. In other words, treatment not only slowed or prevented further retinal degeneration, it also improved the cellular structure and function of the retina, reversing previous degeneration. AAV8.CDHR1 gene therapy restored outer retinal structure in the Cdhr1^{-/-} mouse model with restoration of the ellipsoid zone and photoreceptor outer segments as seen on optical coherence tomography imaging. This finding could explain the functional improvements since the photoreceptor outer segment houses key proteins (such as rhodopsin) which mediate phototransduction (i.e. convert light energy into electrical potentials).

Accordingly, in a first aspect the invention provides a method of treating, preventing or reversing retinal degradation in a subject in need thereof, the method comprising administering to the subject a vector that expresses a Cadherin-related family member 1 (CDHR1) polypeptide.

In a further aspect the invention provides gene therapy vector that expresses a CDHR1 polypeptide.

In a further aspect the invention provides host cell that produces the gene therapy vector.

In a further aspect the invention provides method for production of the gene therapy vector, the method comprises providing a host cell as described above and culturing the host cell under conditions suitable for the production of the vector.

In a further aspect the invention provides a pharmaceutical composition comprising the gene therapy vector.

In a further aspect the invention provides the gene therapy vector or the pharmaceutical composition for use in a method of treating, preventing or reversing retinal degeneration.

In a further aspect the invention provides the use of the gene therapy vector according in the manufacture of a medicament for of treating, preventing or reversing retinal degeneration in a subject.

The disclosure will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the invention which is defined by the claims. All documents cited herein, whether supra or infra, are expressly incorporated by reference in their entirety.

The present disclosure includes the combination of the aspects and features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “a vector” includes two or more such entities. In general, the term “comprising” is intended to mean including but not limited to. In some embodiments of the invention, the word “comprising” is replaced with the phrase “consisting of” or the phrase “consisting essentially of”. The term “consisting of” is intended to be limiting. The term “consisting essentially of” should be understood to mean that the sequence comprises no additional sequence units or elements that materially affect the function of the sequence element.

Section headings are used herein for convenience only and are not to be construed as limiting in any way.

### DESCRIPTION OF THE FIGURES

FIG. 1. Genotyping of Cdhr1 mutant mice. (A) Genotyping primers illustrated against the genomic sequence of Cdhr1 in the C57BL6J strain. In mice, the Cdhr1 gene lies on the reverse strand of chromosome 10 with 17 coding exons, indicated in yellow. WT primer pairs are generated against an intronic region downstream of the 17th coding exon, exploiting a single nucleotide polymorphism in C57BL/6J mice. The position of the knockout reverse primer is indicated, with the forward primer targeted against the PGK Neo-selectable marker (not shown in the wildtype sequence). (B) Agarose gel electrophoresis (1%) showing amplicons with original Jackson Laboratory primer pairs (green), and new wildtype primer pair (red). Both primer sets identify wildtype alleles, although only the new primer pair demonstrates an absence of WT amplicon in Cdhr1^{-/-} mice on the C57BL6J background. This enables accurate genotyping of the Cdhr1 mouse strain. The Jackson WT primer pair produces an amplicon in Cdhr1^{-/-} mice since the targeted sequence has likely reinserted elsewhere in the genome.

FIG. 2. Immunohistochemical and biochemical confirmation of the absence of Cdhr1 protein in Cdhr1^{-/-} mice. (A) Immunostaining with three anti-CDHR1 antibodies identifies Cdhr1 at the base of the photoreceptor outer segments in C57BL6J mice, not present in Cdhr1^{-/-} mice from within the experimental colony. (B) Western blot identifies full-length (FL) Cdhr1 ˜120 kDa, and a more abundant C-terminal fragment ˜25 kDa in C57BL6J whole retinal lysate. Less abundant bands of the same size are seen in Cdhr1^{+/-} heterozygotes. Cdhr1 protein is absent in Cdhr1^{-/-} retinal lysates and lysate of cultured HEK293T cells (NCT). The loading control labels mouse GAPDH.

FIG. 3. Retinal measurements derived from OCT imaging. Longitudinal OCT imaging in Cdhr1^{-/-} and C57BL6J mice demonstrates loss of the ellipsoid band by 6-months of age in Cdhr1^{-/-} mice with progressive outer retinal thinning. Longitudinal short-wavelength autofluorescence (SW-AF) imaging shows the presence of autofluorescent dots at 3-months of age and at later timepoints, accompanied by progressive retinal vascular constriction which is typical of photoreceptor degeneration.

FIG. 4. Longitudinal in vivo multimodal retinal imaging characteristics in Cdhr1^{-/-} and C57BL6J mice to 15 months. Upper panel: Red dots on the SLO NIR reflectance image identify the retinal loci at which thickness measurements are taken in each eye. The bars on the OCT B-scan label the limits of measurement for inner retinal thickness (green bar), photoreceptor layer thickness (blue bar), and ellipsoid zone to RPE (red bar). Lower panel: Longitudinal outer (upper) and inner (lower) retinal thickness measurements in Cdhr1^{-/-} and C57BL6J mice. Progressive outer retinal thinning is seen in Cdhr1^{-/-} mice with a significant difference between 1- and 3-months (P<0.0001; two-way ANOVA), but not 2-months. Outer retinal thinning is seen only at 12-months in C57BL6J mice due to senescence. There is mild inner retinal thinning of both Cdhr1^{-/-} and C57BL6J mice with age, although the effect size is small.

FIG. 5. Longitudinal in vivo optical coherence tomography retinal imaging characteristics in Cdhr1^{-/-} mice. (A) Optical coherence tomography (OCT) retinal imaging studies identified an indistinct ellipsoid zone and photoreceptor outer segment layer in Cdhr1^{-/-} mice at 1 month; neither are discernible at 6 months. Progressive thinning of the outer retina is seen at 6 and 12 months (additional timepoints shown in FIG. 3). Thickening of the innermost hyperreflective band is seen in Cdhr1^{-/-} mice at 1 month, also shown in panel B. (B) OCT reflectivity profiles confirm outer retinal thinning at 1 month in Cdhr1^{-/-} mice. The EZ reflection is diminished with reduced width and depth of the adjacent OS band, indicated by coloured boxes on the reflectivity profile. At 12 months, the outer retina is thinner and ELM and EZ are absent in Cdhr1^{-/-} mice. (C) Photoreceptor layer thickness measurements on OCT imaging in Cdhr1^{-/-} mice are reduced at 1 month compared to wildtype controls (mean 100.2 μm vs 85.6 μm). Data points represent measurements from the superior retina only. Repeated measures two-way ANOVA for overall effect of genotype on photoreceptor layer thickness: F_{1,152}=1754, p<0.0001. Progressive outer retinal degeneration is seen in Cdhr1^{-/-} mice to 15 months (p<0.0001, two-way ANOVA). In C57BL6J mice, the outer retina is not significantly thinned until 12 months of age (p=0.0006; two-way ANOVA). (D) The inner retina is thickened in Cdhr1^{-/-} mice at 1-month of age compared to wildtype with mild progressive thinning to 15 months. (E) Photoreceptor layer thinning progresses more quickly at inferior when compared to superior retinal locations in Cdhr1^{-/-} mice as shown across 8 retinal loci. Repeated measures two-way ANOVA for overall effect of retinal location on photoreceptor layer thickness in Cdhr1^{-/-} mice: F_{7,408}=20.17, P<0.0001. A direct comparison of superior and inferior retinal thickness measurements across age groups Cdhr1^{-/-} cohort identifies significant inferior retinal thinning from 2-9 months (p<0.05 at all timepoints; two-way ANOVA).

FIG. 6. Ellipsoid zone to retinal pigment epithelium measurements in Cdhr1^{-/-} and C57BL6J mice to 3 months. (A) Mean and SD of ellipsoid zone to RPE measurements (which include the photoreceptor outer segment length) for all retinal locations identified a significant difference between Cdhr1^{-/-} and C57BL6J mice over 3 months (P<0.0001; two- tailed, unpaired t-test with t-test estimation plot shown in (B). (C) Individual measurements plotted by retinal location show no significant change in retinal thickness measurement over 3 months in Cdhr1^{-/-} mice on multiple comparison two-way ANOVA. The dashed line indicates average retinal measurements in C57BL6J mice at 3 months for comparison. (D) An increase in ellipsoid zone to RPE measurements are seen in C57BL6J mice which was not statistically significant at most retinal locations.

FIG. 7. Representative OCT images and reflectivity profiles from the same eye at 6 months showing inferior outer retinal thinning in Cdhr1^{-/-} mice. This shows a potential effect of light toxicity, although the effect size is small.

FIG. 8. Dark-adapted electroretinography responses in Cdhr1^{-/-} and C57BL6J mice. (A) Representative dark-adapted and light-adapted electroretinography responses between Cdhr1^{-/-} and C57BL6J mice showing attenuation of responses over 12 months (B) Mean ERG responses across all cohorts across the dark-adapted luminance series showing reduction of both A-wave and B-wave amplitudes at all timepoints in Cdhr1^{-/-}mice. Plots represent mean+/−SEM indicated by shaded areas for each aged cohort. FIG. 9. Dark-adapted electroretinography luminance series in Cdhr1^{-/-} and C57BL6J mice. A-wave and B-wave responses were progressively diminished in Cdhr1^{-/-} mice over 15 months, compared to wildtype. Traces represent mean +/−95% confidence interval. Darker lines represent C57BL6J mice and lighter ones Cdhr1^{-/-} mice.

FIG. 10. Light-adapted and flicker ERG responses in Cdhr1^{-/-} and C57BL6J mice by age. A significant difference between genotype groups is identified at 1 month (Two-way ANOVA; p<0.0001 for all tests). Improvement of cone responses reduces the size of the effect at 2-6 months, except in the single-flash luminance test at 10 cd·s/m² which remains significant at all timepoints (p<0.0001). Reduction in flicker responses in C57BL6J mice is seen at 15 months of age. Plots represent mean+/−standard deviation. Darker bars represent C57BL6J mice and lighter ones Cdhr1^{-/-} mice.

FIG. 11. Functional testing by longitudinal dark-adapted electroretinography in Cdhr1^{-/-} and C57BL6J mice. (A) Representative raw dark-adapted ERG traces at 1, 6 and 12 months. (B) Scotopic B-wave responses at 0.01 cd·s/m² were significantly reduced in Cdhr1^{-/-} mice. Repeated measures two-way ANOVA for overall effect of genotype F_{1.91}=182.7, P<0.0001. Responses did not differ significantly at 2- and 6-month timepoints. (C) Scotopic A-wave responses at 10 cd·s/m² were significantly reduced in Cdhr1^{-/-} mice (p<0.0001 for all timepoints). Repeated measures two-way ANOVA for overall effect of genotype on A-wave amplitude: F_{1.92}=554.8, P<0.0001. (D) Scotopic B-wave responses at 10 cd·s/m² were significantly reduced in Cdhr1^{-/-} mice (P<0.0001, except at 2- and 6-months). Repeated measures two-way ANOVA for overall effect of genotype on B-wave amplitude: F_{1.92}=136.6, P<0.0001. Mean and SEM across representative flash intensities are presented, with full datasets presented in FIGS. 8 & 9.

FIG. 12. Functional testing by longitudinal light-adapted electroretinography in Cdhr1^{-/-} and C57BL6J mice. (A) Representative raw ERG traces at 1, 6 and 12 months showing single-flash (above) and 20 Hz flicker responses (below) at 10 cd·s/m² (B) Photopic single-flash response responses were significantly reduced in Cdhr1^{-/-} mice (P<0.05 for all timepoints). Repeated measures two-way ANOVA for overall effect of genotype on photoptic single-flash amplitude: F_{1.92}=182.8, p<0.0001. (C) Photopic 20 Hz flicker responses were significantly reduced overall in Cdhr1^{-/-} mice. Repeated measures two-way ANOVA for overall effect of genotype on photopic flicker responses: F_{1.92}=89.42, P<0.0001. However, responses did not differ by genotype at the 3- and 6-month timepoints on Šidák's multiple comparisons test at both flash intensities. Mean and SEM across representative flash intensities are presented, with full datasets presented in FIG. 10.

FIG. 13. Relative structural degeneration and functional decline over time in Cdhr1^{-/-} mice. (A) Mixed rod-cone (10 cd·s/m²) A-wave responses on dark-adapted ERG normalised to C57BL6J responses at 1 month shows severe functional deficits at 1-month (62% reduction) when compared to outer retinal thickness measurements on OCT imaging in (C). (B) Normalised light-adapted 20 Hz flicker responses exhibited severe deficits in cone function (70% reduction) in Cdhr1^{-/-} mice at 1 month, with improvement of responses to 3 months, followed by decline. (D) OCT measurements of ellipsoid zone to RPE, normalised to C57BL6J at 1 month shows stable retinal measurements to 3 months. The ellipsoid band is lost in Cdhr1^{-/-} mice at later timepoints.

FIG. 14. CDHR1 expression plasmids. (A) All constructs are packaged in AAV2 ITRs. The full-length human CDHR1 transgene is driven by the human rhodopsin kinase promoter for expression in rod and cone photoreceptor cells. A Kozak consensus sequence drives translation initiation. (B,C) The WPRE and Ex/In/Ex sequences are cis-acting enhancing sequences that have been inserted into two plasmids. Constructs (A) and (C) were selected for AAV packaging to permit a comparison of a minimally enhancing to the maximally enhancing construct.

FIG. 15. AAV8.CDHR1 vector production. (A) SDS-PAGE of constructs A and C from FIG. 14 showing pure AAV preparations with AAV capsid proteins VP1, VP2, VP3 forming bands at ˜87 kDa, ˜73 kDa, and ˜61 kDa without contaminants. (B,C) Quantitative PCR, suing primers directed against the CDHR1 transgenes was used to titres the two AAV8.CDHR1 preparations, achieving high titres as indicated in (D).

FIG. 16. CDHR1 in vivo expression studies in Cdhr1^{-/-} mice. Both AAV8.CDHR1 vectors were delivered by sub-retinal injection at 4-6 weeks of age at a dose of 1.5×10⁹ genome copies, delivered under the superior retina of the right eye. The left eye was used as uninjected control. Eyes were harvested after 4-6 weeks, fixed, cryosectioned and immunostained according to a standardised protocol using primary anti-CDHR1 antibodies.

FIG. 17. Subretinal injection used for downstream in vivo assessments. Under general and topical anaesthesia, mice underwent ab externo sub-retinal injection using an operating microscope and contact lens retinal viewing system with 1.5 μl of vector delivered under the superior retina, achieving a surgical superior hemiretinal detachment.

FIG. 18. Expression of CDHR1 following subretinal delivery of AAV8.GRK1.CDHR1.pA. Immunohistochemical analysis identified expression of CDHR1 within the photoreceptor inner segments and around photoreceptor nuclei (outer nuclear layer) at a dose of 1.5×10⁹. In un-injected control eyes, CDHR1 was not identified. In the inferior retina of treated eyes, less pronounced CDHR1 expression was identified within the inner segments. CDHR1 was identified using primary anti-CDHR1 antibodies targeted against epitopes within the n-terminal cadherin repeats and the extreme c-terminus, supporting the expression of full-length CDHR1.

FIG. 19. Expression of CDHR1 following subretinal delivery of AAV8.GRK1.In.CDHR1.WPRE.pA. CDHR1 expression was similarly identified in the inner segments of photoreceptors using anti-CDHR1 antibodies. Immunostaining was not identified in uninjected control eyes, and in control sections that were not incubated in primary anti-CDHR1 antibodies.

FIG. 20. Expression of CDHR1 in cone photoreceptors following subretinal delivery of AAV8.GRK1.CDHR1.pA. Co-localisation of CDHR1 with peanut agglutinin conjugated to fluorescein proves expression of the CDHR1 transgene within cone photoreceptors in Cdhr1^{-/-} mice.

FIG. 21. CDHR1 in vivo dosing studies in Cdhr1^{-/-} and C57BL6J mice. Overview of study design that seeks to evaluate the maximum tolerated dose of both AAV8.CDHR1 vectors in both Cdhr1^{-/-} and C57BL6J mice. 112 mice underwent paired injections of AAV8 vector in one eye and PBS-PF68 0.001% vehicle control in the fellow eye, at a dosing range from 7.5×10⁶ to 7.5×10⁹ (3 log units). Mice were injected at 4-6 weeks of age, with retinal imaging using OCT undertaken at 4-6 weeks post-injection. The maximum tolerated dose was identified by the absence of retinal thinning in the superior retina of injected eyes in both Cdhr1^{-/-} and C57BL6J mice. If retinal thinning was excluded, Cdhr1^{-/-} mice underwent electroretinography 4-weeks after the retinal imaging studies in order to determine if an early therapeutic effect was present. This enabled a priori power calculations to adequately power a prospective study with respect to functional outcome measures, and to further guide dose selection (if more than one dose was shown to be safe on retinal imaging studies).

FIG. 22. AAV8.GRK1.CDHR1.pA dosing study in Cdhr1^{-/-} mice. Retinal thickness measurements 4-weeks post-subretinal injection show retinal thinning affecting the superior retinas of some treated eyes at a dose of 1.5×10⁹ (P=0.025 versus 7.5×10⁸). This was not observed at lower doses (i.e. 7.5×10⁸ or lower). Normalised retinal thickness measurements between paired eyes show mild thinning of the inferior retina at a dose of 1.5×10⁹ 2 of 7 eyes.

FIG. 23. AAV8.GRK1.CDHR1.pA dosing study in C57BL6J mice. Retinal thickness measurements 4-weeks post-subretinal injection show retinal thinning in the superior retina at a dose of 7.5×10⁹, and some retinas at doses of 1.5×10⁹ to 7.5×10⁸. This was not observed at a dose of 1.5×10⁸ or lower, where inferior retinas did not demonstrate thinning. This is shown also using normalised data comparing AAV with PBS injected paired eyes (i.e. no retinal thinning at a dose of 1.5×10⁸ or lower).

FIG. 24. AAV8.GRK1.In.CDHR1.WPRE.pA dosing study in Cdhr1^{-/-} mice. Retinal thickness measurements 4-weeks post-subretinal injection show a dose-dependent thinning in the superior retina at a dose of 7.5×10⁸, persisting with a dose of 1.5×10⁷. Normalised data comparing AAV and PBS-injected paired eyes shows mild retinal thinning at a dose of 1.5×10⁷.

FIG. 25. AAV8.GRK1.In.CDHR1.WPRE.pA dosing study in C57BL6J mice. In wildtype mice, doses of 7.5×10⁶ to 1.5×10⁷ did not show any retinal thinning following subretinal injection; also seen on normalisation of the data between AAV- and PBS-injected control eyes. There was no significant difference between 7.5×10⁶ to 1.5×10⁷.

FIG. 26. Dark-adapted electroretinography following AAV8.GRK1.CDHR1.pA injection in Cdhr1^{-/-} mice (pilot as part of vector dosing study). A-wave amplitudes (representing photoreceptor responses) at different light stimulus intensities are plotted for a range of AAV doses (1.5×10⁹ to 1.5×10⁸), and compared against PBS-injected eyes, uninjected eyes (natural history) and wildtype eyes. This shows a beneficial effect on mean responses in both the 1.5×10⁸ and 7.5×10⁸ groups which showed no retinal thinning on OCT imaging 4-weeks previously. These beneficial functional effects were statistically significant using two-way ANOVA as indicated on the chart. Note that the natural history data and PBS-injected controls show well matched responses (indicating no functional effect following retinal detachment).

FIG. 27. Light-adapted electroretinography following AAV8.GRK1.CDHR1.pA injection in Cdhr1^{-/-} mice (pilot as part of vector dosing study). Dark-adapted flicker responses were significantly improved in AAV-injected eyes versus PBS injected eyes and uninjected control eyes in both the 1.5×10⁸ and 7.5×10⁸ groups (P<0.05 using two-way ANOVA for both groups against control groups). Light-adapted flicker responses were improved in both the 1.5×10⁸ and 7.5×10⁸ groups although this was not statistically significant at this timepoint at these doses.

FIG. 28. A priori power calculation used to determine the sample size for prospective, open-label controlled study of AAV8.CDHR1 gene therapy. Based on dark-adapted A-wave responses, independent group means, with alpha=0.05 and a one-tailed test, it was calculated that 26 mice were required in the Cdhr1^{-/-} group at a dose of 1.5×10⁸ to achieve 95% power. The dose of 1.5×10⁸ was designated as the high-dose arm, and 1.5×10⁷ was designated as the low dose arm. The lower dose group was not supported by a power calculation since a pilot study had not been undertaken at that dose. Moreover, ERG analysis was not undertaken on the wildtype group at the same doses, although were shown on OCT imaging not to exhibit a toxic effect.

FIG. 29. Experimental design for longitudinal evaluation of AAV8.CDHR1 gene therapy. (a) Prospective, open-label, longitudinal controlled study, comparing the effect of sub-retinal AAV with PBS. 103 mice were included in the study, 24-28 per dosing group. Cdhr1^{-/-} mice were used to determine safety and efficacy and C57BL6J mice to determine safety. Mice were injected after weaning (3-4 weeks of age), followed by serial electroretinography-ERG) at 2,4,6 and 8-months post-injection, and OCT imaging at 1,6, and 12 months post-injection. (b) Study schedule showing additional timepoints. ERG at 8, 10 and 12 months, optomotor response testing (OMR) of visual behaviour at 18 to 22 months, and OCT imaging at 12 and 18 months were conducted in high dose groups only.

FIG. 30. OCT imaging 4 weeks post injection with AAV8.GRK1.CDHR1.pA in Cdhr1^{-/-} and C57BL6J mice. In C57BL6J wildtype mice (upper) and Cdhr1^{-/-} mice (lower), there was no apparent retinal thinning between AAV- and PBS-injected eyes at equivalent retinal loci, at either high or low dose. These data indicated that at 1-month after AAV injection, there was no evidence of retinal toxicity at high or low dose in either group. Mean values are annotated numerically, with each measurement presented as an individual data-point. Error bars represent standard deviation. Two-way ANOVA compares AAV- and PBS-injected control eyes at both retinal locations (superior and inferior), at both doses (P>0.05 for all comparisons).

FIG. 31. OCT imaging 6-months post injection with AAV8.GRK1.CDHR1.pA in Cdhr1^{-/-} and C57BL6J mice. In C57BL6J mice (upper panel), there was no difference in retinal thickness measurements at equivalent superior retinal loci between AAV- or PBS-injected eyes at either high-dose (P=0.15) and low-dose (P>0.99). In Cdhr1^{-/-} eyes, retinal thickness measurements were significantly greater (76.6 um versus 49.7 um) in AAV-injected eyes versus PBS-injected eyes (two-way ANOVA, p<0.0001). Furthermore, retinal thickness measurements were also significantly greater at inferior retinal loci (58.0 versus 52.1 um; p=0.012), although the effect size was smaller. At the low dose, there was a slight increase in superior retinal thickness measurements in AAV-injected eyes (49.7 versus 45.3; P=0.028), although no difference at inferior retinal locations between groups (P=0.66).

FIG. 32. OCT imaging 12-months post injection with AAV8.GRK1.CDHR1.pA in Cdhr1^{-/-} and C57BL6J mice. In C57BL6J mice, there was no difference in retinal thickness at both superior and inferior retinal locations between AAV- and PBS-injected eyes (upper). In Cdhr1−/− mice, superior retinal thickness measurements remained greater in AAV- versus PBS-injected control eyes (70.2 um versus 29.3 um; P<0.0001), with a smaller therapeutic effect seen inferiorly (P=0.0032).

FIG. 33. OCT imaging appearances 6 and 12-months post injection with AAV8.GRK1.CDHR1.pA in Cdhr1^{-/-} mice. OCT imaging 6-months follow AAV injection shows preservation of the outer nuclear layer representing photoreceptor cell bodies, indicating prevention of photoreceptor cell death (OPL-RPE measurements are indicated by red bars). White arrows indicate the OCT layer representing photoreceptor outer segments, which are not present in Cdhr1^{-/-} mice at any timepoint since the absence of the gene results in disordered outer segments. Moreover, at 12 months, the ellipsoid zone band has become wider and more reflective in AAV injected eyes, whilst the outer segment band (white arrow) is maintained. In PBS injected eyes, progressive retinal degeneration is seen with loss of photoreceptors as seen as thinning of the outer nuclear layer, as demonstrated in the mouse natural history data (FIGS. 4 & 5).

FIG. 34. OCT reflectivity profiles post injection with AAV8.GRK1.CDHR1.pA in Cdhr1^{-/-} mice. At 1-month post-injection, both AAV and PBS injected control eyes show an ellipsoid band which appears as a highly reflective peak (arrow), with little or no distinct hyporeflective band following it consistent with the OCT appearance of significantly shortened outer segments. At 6 months, AAV injected retinae show regeneration of the ellipsoid band (arrow), which appears more reflective (indicating improved photoreceptor organisation versus 1-month), and the hyporeflective band that follows indicates regeneration of photoreceptor outer segments. In PBS injected eyes, the ellipsoid band is lost by 6 months, consistent with continued degeneration of photoreceptors and their outer segments. It is notable that outer segment regeneration is also seen at inferior retinal locations in some AAV injected eyes, as shown in the lower panel.

FIG. 35. Extent and timing of outer segment regeneration in Cdhr1^{-/-} mice following AAV gene therapy. Upper panel shows a representative OCT image taken from an AAV injected eye, 6-months after sub-retinal injection with AAV8.GRK1.CDHR1.pA. The internal measurement callipers as part of the Heidelberg imaging software were used to identify the length across which outer segment regeneration was identifiable (measuring >10 um parallel to the A-scan). This analysis showed that outer segment regeneration is identifiable as early as 1-month post injection (p=0.001 versus PBS-injected control eyes). At 6-months, regeneration within the group is more frequent and more extensive (P<0.0001 versus control eyes). Note that an outer segment >10 um in length is not identifiable in Cdhr1^{-/-} mice, or following PBS injection.

FIG. 36. Dark-adapted electroretinography following high dose (1.5×10⁸) AAV8.GRK1.CDHR1.pA injection in Cdhr1^{-/-} mice. Plots for all ERG traces in FIG. 36-43 represent mean +/−95% confidence intervals. Statistical analysis is presented on each chart, both overall across the light-intensities or group of tests presented on any chart, and for individual tests, where relevant. From 2-months post-injection, dark-adapted A-wave responses were significantly better in AAV- versus PBS-injected eyes (p<0.0001). The therapeutic benefit between AAV- and paired PBS-injected eyes increased at each successive timepoint (P<0.0001 throughout). B-wave responses (representing bipolar cells) were greater in AAV-treated eye, but not significant at 2 months post-injection (P=0.058). However, after 4-months, B-wave responses were significantly improved (P=0.015), and continue to show increasing benefit at each successive timepoint.

FIG. 37. Light-adapted electroretinography following high dose (1.5×10⁸) AAV8.GRK1.CDHR1.pA injection in Cdhr1^{-/-} mice. Across five flicker and light-adapted tests, AAV-injected retinas demonstrated improved responses in comparison to paired PBS-injected control eyes as early as 2-months (P<0.0001, overall). However, at the earliest timepoint, only dark-adapted flicker was significantly improved in AAV injected eyes (P<0.0001). At each successive timepoint, there was a greater benefit seen in AAV-injected eyes versus controls, which by 10-months post-injection was significant for all ERG tests. DA—dark-adapted; LA—light-adapted

FIG. 38. Dark-adapted electroretinography following high dose (1.5×10⁸) AAV8.GRK1.CDHR1.pA injection in C57BL6J mice. A- and B-wave responses were not found to be significantly different in AAV- and PBS-injected eyes at 6-months post-injection (P=0.65 for A-wave and P=0.47 for B-wave). At 10 months, there was a slight decrease in A-wave responses in AAV-injected eyes (p=0.0015) but this was insufficient to be significantly different at any single light intensity. However, there was no difference in B-wave responses (P=0.057).

FIG. 39. Light-adapted electroretinography following high dose (1.5×10⁸) AAV8.GRK1.CDHR1.pA injection in C57BL6J mice. No difference was identified between AAV- and PBS-injected control eyes in any light-adapted or flicker ERG test at any timepoint. This strongly suggests that high dose AAV does not have any detrimental effect on cone function in C57BL6J mice.

FIG. 40. Dark-adapted electroretinography following low dose (1.5×10⁷) AAV8.GRK1.CDHR1.pA injection in Cdhr1^{-/-} mice. A beneficial effect on A-wave (P=0.027) and B-wave responses (P=0.03) of AAV- over PBS-injected control eyes was seen with low dose AAV. However, the effect size was much smaller than in the high-dose group (FIG. 36).

FIG. 41. Light-adapted electroretinography following low dose (1.5×10⁷) AAV8.GRK1.CDHR1.pA injection in Cdhr1^{-/-} mice. There was no significant difference between AAV- and PBS-injected eyes, except a small statistical benefit overall at 6 months (P=0.017). However, no single test was statistically significant at any timepoint.

FIG. 42. Dark-adapted electroretinography following low dose (1.5×10⁷) AAV8.GRK1.CDHR1.pA injection in C57BL6J mice. There was no significant difference between A-wave and B-wave responses in AAV- and PBS-injected eyes at 4 and 6 months. A difference at 2-months was attributed to a faulty electrode which were subsequently alternated between treated and control eyes all subsequent experiments.

FIG. 43. Light-adapted electroretinography following low dose (1.5×10⁷) AAV8.GRK1.CDHR1.pA injection in C57BL6J mice. There was no significant difference between AAV- and PBS-injected eyes at 4- and 6-months post injection (P=0.59 at 6 months). Moreover, there was no significant difference in any individual ERG test at any timepoint between groups.

FIG. 44. Data from the international clinical CDHR1 study. Age distribution of individuals with CDHR1-associated retinal degeneration shows that the majority are of working age. Natural history data show that for individuals with biallelic null variants in CDHR1, severe visual loss occurs in 100% of individuals before the age of retirement.

FIG. 45. Raw dark- and light-adapted ERG traces showing rescue of photoreceptor function in Cdhr1−/− mice at 12 months post-injection of high-dose AAV8.GRK1.CDHR1.pA. Raw ERG traces recorded simultaneously from the same mouse 12-month post-injection show preserved responses in the AAV- (right eye) versus the PBS-injected eye (left eye). Note the presence of the A-wave on dark-adapted ERG in AAV-treated eyes only, indicating preserved rod and cone photoreceptor function, which are absent in the PBS-injected eye. B-wave responses are also larger and oscillatory potentials discernible in AAV-injected eyes only.

FIG. 46. Dark- and light-adapted ERG following high-dose AAV8.GRK1.CDHR1.pA in Cdhr1−/− mice to 12 months post-injection. Rescue of rod and cone photoreceptor function in AAV-treated eyes is demonstrated as early as 2-months post-injection, with the therapeutic effect size increasing at each successive timepoint. Plots represent mean+95% C.I. Shapiro-Wilk indicated parametric datasets; P values were derived from two-way ANOVA for effect of treatment with Šídák's multiple comparison test for further comparisons.

FIG. 47. Dark- and light-adapted ERG following low-dose AAV8.GRK1.CDHR1.pA in Cdhr1−/− mice to 6 months post-injection. A statistical benefit of AAV- over PBS-injected eyes was demonstrated in A-wave responses from 4-months post-injection, and B-wave responses from 6-months post-injection. Light-adapted single-flash and flicker responses were improved overall, although not significantly different for individual tests. Plots represent mean+95% C.I. Shapiro-Wilk indicated parametric datasets; P values were derived from two-way ANOVA (for effect of treatment) with Šídák's multiple comparison test used for post-hoc analysis of light-adapted responses.

FIG. 48. Cone photoreceptor response kinetics following subretinal injection of AAV8.GRK1.CDHR1.pA to 12 months post-injection. High-dose AAV- shortens A-wave implicit times on light-adapted single flash ERG in Cdhr1−/− mice from 2-months post-injection (P=0.03) with increasing effect at later timepoints (P<0.0001). AAV-injected responses in Cdhr1−/− eyes were no different to those in C57BL/6J at 6-months (P=0.58), although differed at 12-months (P<0.0001). In the low-dose group, responses were shorter in AAV-injected eyes at 6-months (P=0.018), but not at 2-months post-injection (P=0.73). AAV-injection did not affect A-wave implicit times in C57BL/6J mice at any dose or timepoint. Plots represent mean+95% C.I. Shapiro-Wilk indicated parametric datasets; P values are derived from two-way

FIG. 49. Raw light-adapted ERG traces illustrating A-wave implicit times after high dose AAV8.GRK1.CDHR1.pA in Cdhr1−/− mice. Longitudinal ERG traces are presented for the same Cdhr1−/− mouse over 12 months. Increases in response amplitudes at 6 months, consistent with natural history data (Chapter 3). Vertical bars indicate A-wave peaks for AAV-(red) and PBS-(blue) injected eyes. At 2-months post-injection, A-wave implicit times are shorter in AAV-injected eyes, a difference that increases at the 6-month timepoint. At 12-months, A-waves are not clearly discernible in all PBS-injected eyes.

FIG. 50. Dark- and light-adapted ERG following high-dose AAV8.GRK1.CDHR1.pA in C57BL/6J mice to 12 months post-injection. There is no difference in dark- and light-adapted ERG between AAV- and PBS-injected eyes in C57BL/6J mice to 12-months post-injection. Plots represent mean+95% C.I. Shapiro-Wilk indicated parametric datasets; P values are derived from two-way ANOVA for effect of treatment, stated overall between treatment groups at all light intensities. Šídák's multiple comparison test is used to compare individual light-adapted and flicker tests between treatment groups.

FIG. 51. Dark- and light-adapted ERG following low-dose AAV8.GRK1.CDHR1.pA in C57BL/6J mice to 6 months post-injection. Dark- and light-adapted ERG does not detect differences between AAV- and PBS-injected eyes to 6-months post-injection with low-dose AAV8.GRK1.CDHR1.pA. Plots represent mean+95% C.I. Shapiro-Wilk indicated parametric datasets; P values are derived from two-way ANOVA for effect of treatment with Šídák's multiple comparison test. The recorded difference between groups at 2-months was due to a faulty electrode which were subsequently alternated between AAV- and PBS-injected eyes at later timepoints, and all other ERG assessments.

FIG. 52. Dark-adapted A-wave implicit times following subretinal injection of AAV8.GRK1.CDHR1.pA to 12 months post-injection. Data are plotted from both AAV- and PBS-injected Cdhr1−/− and C57BL/6J mice, at the five highest light intensities on the dark-adapted luminance series. In the high dose group, AAV-injected Cdhr1−/− eyes demonstrated shorter A-wave implicit times compared to PBS-injected eyes (P<0.0001 at all timepoints). This effect was sustained to 12-months. A benefit was not seen in the low dose group. In C57BL/6J mice, A-wave implicit times were no different in AAV- and PBS-injected eyes in either dosing group. Plots represent mean+95% C.I. Shapiro-Wilk indicated parametric datasets; P values are derived from two-way ANOVA for effect of treatment with Śídák's multiple comparison test.

FIG. 53. Rescue of photopic and scotopic optomotor reflexes to 21-months post-injection of high-dose AAV.GRK1.CDHR1.pA in Cdhr1−/− mice. Upper left panel shows responses from the AAV-injected right eye isolated by anticlockwise rotation of the OKN drum under photopic conditions. A smooth pursuit head movement (optomotor response) in the direction of rotation of the drum is maintained for 2 seconds, indicating visualisation and fixation of the vertical grating. Upper right panel shows an absence of optomotor response from the PBS-injected control eye. Lower left panel shows significantly greater OMR responses in AAV- versus PBS-injected control eyes at both fast (15 s/rotation; P<0.0001) and slow (30 s/rotation; P<0.0001) OKN drum speeds under photopic conditions (1000 lux) at 19-months post-injection, with beneficial effects also demonstrated under scotopic conditions (0.01 lux) at 21-months post-injection at both rotation speeds (P<0.0001), shown in the lower right panel. Statistical tests presented are two-way ANOVA for the effect of treatment.

FIG. 54. Preservation of photopic and scotopic optomotor reflexes to 22-months post-injection following high-dose AAV8.GRK1.CDHR1.pA in C57BL/6J mice. Photopic (1000 lux) and scotopic (0.01 lux) optomotor responses were no different between AAV- and PBS-injected eyes in C57BL/6J mice at 20- and 22-months post-injection, respectively (P=0.25 for photopic, P=0.12 for scotopic testing on two-way ANOVA for the effect of treatment). Moreover, no differences were observed between groups at two speeds of rotation of the OKN drum at either level of illuminance, at a fixed spatial frequency of 0.1 cycles per degree. Plots present mean of three technical replicates. Error bars represent SEM.

FIG. 55. Structure-function correlation in Cdhr1−/− and C57BL/6J mice following CDHR1 gene therapy. Plots represent mean A-wave amplitudes on dark adapted, single-flash ERG at 10 cd·s/m² taken at 2-, 6- and 12-months post-injection and mean superior photoreceptor layer thickness measurements on OCT imaging at 1-, 6- and 12-months post-injection. Simple linear regression lines are plotted for natural history data (dashed lines, Chapter 3), AAV-injected eyes (solid lines), and PBS-injected eyes (dotted lines). In C57BL/6J mice, all three groups demonstrate a similar structure-function relationship. In Cdhr1−/− mice, structural and functional degeneration is slowed in AAV-injected eyes compared to natural history data and PBS-injected eyes, as exemplified by differences in the slopes. The dashed vertical line represents the floor of retinal thickness measurements.

FIG. 56. Functional rod and cone photoreceptor rescue in Cdhr1−/− mice relative to C57BL/6J following high dose AAV8.GRK1.CDHR1.pA. Rescue of rod and cone responses are well matched in Cdhr1−/− mice to 12-months post-injection of high-dose AAV8.GRK1.CDHR1.pA. Note that normalised cone responses also fall below those of rods in PBS-injected Cdhr1−/− eyes relative to C57BL/6J. All responses are normalised to age-matched and treatment-matched C57BL/6J mice. Peak normalised responses at 5 months of age are consistent with Cdhr1−/− natural history data (Chapter 3). Cone responses are derived from light-adapted flicker responses at 10 cd·s/m² and rod responses from dark-adapted single-flash B-wave responses at 0.01 cd·s/m².

FIG. 57. Photoreceptor layer thickness measurements on OCT imaging following high-dose AAV8.GRK1.CDHR1.pA to 18 months post-injection in Cdhr1−/− and C57BL/6J mice. AAV-injected Cdhr1-/- eyes demonstrate a slowing of photoreceptor degeneration as demonstrated by preserved photoreceptor layer thickness measurements at 6-months post-injection (P<0.0001), and at all later timepoints. The rescue effect is marked superiorly where the bleb is delivered, and mild inferiorly at 6- and 12-months post-injection. In C57BL/6J mice, there is no significant retinal thinning in AAV-injected eyes, except in the superior retina at 18-months. Plots represent mean+95% C.I. Shapiro-Wilk indicated parametric datasets; P values are derived from two-way ANOVA (effect of treatment) with Šídák's multiple comparison test.

FIG. 58. Structural superior-to-inferior therapeutic gradient following high-dose AAV8.GRK1.CDHR1.pA in Cdhr1−/− mice. Representative OCT images from two mice 18-months post-injection showing a superior to inferior gradient of structural preservation that is lost (dashed red line) in the peripheral inferior retina where retinal structure appears similar to a PBS-injected control eye at the same timepoint.

FIG. 59. Morphological outer retinal features on OCT imaging following high-dose AAV8.GRK1.CDHR1.pA to 18 months post-injection. Raw OCT images from a single Cdhr1−/− mouse show preservation of the photoreceptor layer in the superior retina following AAV-injection to 18-months post-injection, with severe outer retinal degeneration in the paired PBS-injected eye at the same loci. Reflectivity profiles highlight congenitally shortened OS at 1-month in both treatment groups which lengthened and become defined at 6- and 18-months post injection in AAV-, but not PBS-injected eyes. Increased reflectivity of the ellipsoid zone was seen in AAV-injected eyes from 6-months and later timepoints, which appeared iso-reflective to the ELM at 1-month in both groups. In PBS-injected eyes, the EZ and OS were absent at 6-months, and near total outer retinal degeneration with loss of ONL is seen at 18-months. The ELM was preserved to 18-months post-injection in AAV-injected eyes, and absent from 12-months in PBS-injected eyes. Coloured bars on reflectivity profiles indicate photoreceptor layer thickness measurements which diverge in treatment groups. Reflectivity profiles were generated from the presented raw OCT images in the axis of the OCT A-scan using ImageJ.

FIG. 60. Multimodal retinal imaging findings in a representative Cdhr1−/− mouse 18-months post-injection with AAV.GRK1.CDHR1.pA. IR reflectance (A), SW-AF (B), NIR-AF (C), and OCT images (D) are presented for paired AAV- (right) and PBS-injected eyes (left). Dashed lines represent the approximate limits of the sub-retinal bleb. In AAV-injected eyes, en face retinal imaging techniques demonstrate an absence of features of retinal degeneration in the superior retina, although mild degenerative features are seen inferiorly (A-C). In PBS-injected eyes, severe generalised retinal degeneration is seen with a distinct peripapillary distribution with degeneration, but not loss, of the RPE. OCT images from equivalent retinal loci (as indicated by green line in A) show a clear rescue effect in AAV-injected eyes with preservation of ONL, EZ and OS regeneration. PBS-injected eyes exhibit severe outer retinal degeneration.

FIG. 61. Timing and extent of outer segment regeneration on OCT imaging following high dose AAV.GRK1.CDHR1.pA in Cdhr1−/− mice. Upper: Measurement of the radial extent of OS regeneration by an observer blinded to the treatment allocation demonstrated evidence of OS regeneration at 1-month post-injection, which increased in extent to 12-months and was sustained to 18-months post-injection (P<0.0001 at all timepoints). Plots represent mean and 95% CI. OS regeneration was not identified in PBS-injected eyes. Lower: OCT images of the superior retina of the same Cdhr1−/− mouse showing regeneration of congenitally shortened OS at 12-months post AAV-injection. The reflectivity of the ellipsoid band appears greater where OS length is greater

FIG. 62. Restoration of outer segment length, alignment and morphology on transmission electron microscopy 21-months after high dose AAV.GRK1.CDHR1.pA in Cdhr1−/− mice. (A) Full-length (˜30 μm), aligned and correctly orientated photoreceptor OS are identified in the superior retina following CDHR1 gene therapy. (B) Elongated, vertically orientated mitochondria are identified in the IS with normal morphology compared to wildtype mice (data not shown). (C) Incisures (arrowheads) in the rod photoreceptor OS are demonstrated, supporting the restoration of the higher-order organisation following CDHR1 gene therapy. (D) Normal morphology of nascent photoreceptor discs with close alignment to the periciliary ridge of the inner segment. Regular spacing of the OS discs throughout the length of the proximal OS is apparent adjacent to the connecting cilium. In the PBS-injected fellow eye of this mouse, photoreceptors and outer segments were not identifiable. Tissue recovery and fixation performed by the author; embedding, sectioning and TEM image capture performed by Dr. Thomas Burgoyne. Scale bars represent 2 μm, 400 nm, 20 nm and 20 nm, respectively. BM—Bruch's membrane; Mt—mitochondrion; CC—connecting cilium.

FIG. 63. Area of RPE degeneration by retinal location in Cdhr1−/− mice 18-months post-injection with high-dose AAV8.GRK1.CDHR1.pA. (A) AAV gene therapy slows down RPE degeneration as seen on NIR-AF imaging; P<0.0001 overall compared to PBS-injected eyes. This effect is most marked in the superior retina of AAV-treated eyes (P<0.0001), although the beneficial effect is also seen inferiorly (P<0.0001). Severe, generalised RPE degeneration is seen in PBS-injected eyes (B). Overall, inferior retinal locations exhibited more RPE degeneration than superior ones, despite superior surgically-induced retinal detachments. The area of degeneration was calculated using Heidelberg imaging software following standardised 55° NIR-AF imaging centred on the optic disc. Shapiro Wilk: non-parametric datasets. Wilcoxon paired signed rank test used.

### DESCRIPTION OF THE SEQUENCES

SEQ ID NO: 1 set forth the 2AAV 5′ITR sequence.
SEQ ID NO: 2 sets forth the sequence of the human rhodopsin kinase promoter (GRK1).
SEQ ID NO: 3 sets forth the sequence of the human rhodopsin promoter (RHOp)
SEQ ID NO: 4 sets forth the chicken beta-actin promoter exon-intron-exon sequence.
SEQ ID NOs: 5 sets forth human CDHR1 nucleic acid sequence.
SEQ ID NOs: 6 sets forth human CDHR1 amino acid sequences.
SEQ ID NO: 7 sets forth the WPRE sequence.
SEQ ID NO: 8 sets forth the bovine growth hormone polyadenylation tail sequence.
SEQ ID NO: 9 set forth the 2AAV 3′ITR sequence.
SEQ ID NO: 10 sets forth the polynucleotide sequence of vector GRK1.CDHR1.pA
SEQ ID NO: 11 sets forth the polynucleotide sequence of vector GRK1.CDHR1.WPRE.pA
SEQ ID NO: 12 sets forth the polynucleotide sequence of vector GRK1.In.CDHR1.WPRE.pA
SEQ ID NO: 13 sets forth the amino acid sequence of CDHR1 extracellular cadherin repeat domain 1.
SEQ ID NO: 14 sets forth the amino acid sequence of CDHR1 extracellular cadherin repeat domain 2.
SEQ ID NO: 15 sets forth the amino acid sequence of CDHR1 extracellular cadherin repeat domain 3.
SEQ ID NO: 16 sets forth the amino acid sequence of CDHR1 extracellular cadherin repeat domain 4.
SEQ ID NO: 17 sets forth the amino acid sequence of CDHR1 extracellular cadherin repeat domain 5.
SEQ ID NO: 18 sets forth the amino acid sequence of CDHR1 extracellular cadherin repeat domain 6.

### DETAILED DESCRIPTION

### Vectors

The invention relates to vectors and gene therapy vectors. A gene therapy vector is any vector suitable for use in gene therapy, i.e. any vector suitable for the therapeutic delivery of nucleic acid polymers into target cells. In the present case, the gene therapy vectors encode a therapeutic gene product, a CDHR1 polypeptide, and can be used to express the product in photoreceptor cells in the retina. The vector may be of any suitable type, such as a plasmid vector or a minicircle DNA. Typically, the vector is a viral vector. The viral vector may, for example, be an adeno-associated virus (AAV), a retrovirus, a lentivirus, a herpes simplex virus, or an adenovirus. Relevant sections of the description relating the AAV derived vectors also apply in the case of vectors derived from other sources, such as those discussed further below.

### AAV Vectors

The vector may comprise a genome from a naturally derived serotype, isolate or clade of AAV or a derivative or one or more functional units thereof. An AAV genome is a polynucleotide sequence which encodes one or more functions needed for production of an AAV viral particle. Naturally occurring AAV viruses are replication-deficient and rely on the provision of helper functions in trans for completion of a replication and packaging cycle. Accordingly, the AAV genome of the vector of the invention is typically replication-deficient.

The AAV genome may be in single-stranded form, either positive or negative-sense, or in double-stranded form. The use of a double-stranded form allows bypass of the DNA replication step in the target cell and so can accelerate transgene expression.

In general, for therapeutic purposes, the only sequences required in cis, in addition to the therapeutic gene is at least one inverted terminal repeat sequence (ITR). In naturally derived AAV, the ITR sequence(s) act in cis to provide a functional origin of replication, and allows for integration and excision of the vector from the genome of a cell. The natural AAV genome also comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV viral particle. Capsid variants are discussed below.

A promoter may be operably linked to each of the packaging genes. Specific examples of such promoters include the p5, p19 and p40 promoters (Laughlin et al., 1979, PNAS, 76:5567-5571). For example, the p5 and p19 promoters are generally used to express the rep gene, while the p40 promoter is generally used to express the cap gene.

In therapeutic AAVs, the cap and/or rep genes may be removed. The removal of the viral genes renders rAAV incapable of actively inserting its genome into the host cell DNA. Instead, the rAAV genomes fuse via the ITRs, forming circular, episomal structures, or insert into pre-existing chromosomal breaks. For viral production, the structural and packaging genes, now removed from the rAAV, are supplied in trans, in the form of a helper plasmid. This is discussed further below. Removal of the cap and/or rep genes provides additional capacity for the insertion of a transgene such as, in the present case, CDHR1. Hence, the gene therapy vectors described herein are recombinant viral vectors.

As is known to the skilled person, AAV viruses occurring in nature may be classified according to various biological systems.

Commonly, AAV viruses are referred to in terms of their serotype. A serotype corresponds to a variant subspecies of AAV which, owing to its profile of expression of capsid surface antigens, has a distinctive reactivity that can be used to distinguish it from other variant subspecies. Typically, a virus having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11, also recombinant serotypes, such as Rec2 and Rec3, identified from primate brain. In vectors of the invention, the genome may be derived from any suitable AAV serotype, such as AAV2 or AAV9.

The capsid may also be derived from any suitable AAV serotype, such as AAV8. Reviews of AAV serotypes may be found in Choi et al. (Curr Gene Ther. 2005; 5(3); 299-310) and Wu et al (Molecular Therapy. 2006; 14(3), 316-327).

Examples of AAV genome sequences that may in some cases be suitable, or of functional sequence units, including ITR sequences, rep or cap genes and regulatory elements, that may in some cases be suitable, may be derived from the following accession numbers: Adeno-associated virus 1 NC_002077.1, AF063497; Adeno-associated virus 2 NC_001401.2; Adeno-associated virus 3 NC_001729.1; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature. The term genetic isolate describes a population of AAV viruses which has undergone limited genetic mixing with other naturally occurring AAV viruses, thereby defining a recognisably distinct population at a genetic level.

Examples of clades and isolates of AAV that may similarly be suitable include:
Clade A: AAV1 NC_002077, AF063497, AAV6 NC_001862, Hu. 48 AY530611, Hu 43 AY530606, Hu 44 AY530607, Hu 46 AY530609
Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AY530624, Hu64 AY530625, Hu13 AY530578, Hu56 AY530618, Hu57 AY530619, Hu49 AY530612, Hu58 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, Hu T70 AY695373, Hu T40 AY695372, Hu T32 AY695371, Hu T17 AY695370, Hu LG15 AY695377,
Clade C: Hu9 AY530629, Hu10 AY530576, Hu11 AY530577, Hu53 AY530615, Hu55 AY530617, Hu54 AY530616, Hu7 AY530628, Hu18 AY530583, Hu15 AY530580, Hu16 AY530581, Hu25 AY530591, Hu60 AY530622, Ch5 AY243021, Hu3 AY530595, Hu1 AY530575, Hu4 AY530602 Hu2, AY530585, Hu61 AY530623
Clade D: Rh62 AY530573, Rh48 AY530561, Rh54 AY530567, Rh55 AY530568, Cy2 AY243020, AAV7 AF513851, Rh35 AY243000, Rh37 AY242998, Rh36 AY242999, Cy6 AY243016, Cy4 AY243018, Cy3 AY243019, Cy5 AY243017, Rh13 AY243013
Clade E: Rh38 AY530558, Hu66 AY530626, Hu42 AY530605, Hu67 AY530627, Hu40 AY530603, Hu41 AY530604, Hu37 AY530600, Rh40 AY530559, Rh2 AY243007, Bb1 AY243023, Bb2 AY243022, Rh10 AY243015, Hu17 AY530582, Hu6 AY530621, Rh25 AY530557, Pi2 AY530554, Pi1 AY530553, Pi3 AY530555, Rh57 AY530569, Rh50 AY530563, Rh49 AY530562, Hu39 AY530601, Rh58 AY530570, Rh61 AY530572, Rh52 AY530565, Rh53 AY530566, Rh51 AY530564, Rh64 AY530574, Rh43 AY530560, AAV8 AF513852, Rh8 AY242997, Rh1 AY530556
Clade F: Hu14 (AAV9) AY530579, Hu31 AY530596, Hu32 AY530597, Clonal Isolate AAV5 Y18065, AF085716, AAV 3 NC_001729, AAV 3B NC_001863, AAV4 NC_001829, Rh34 AY243001, Rh33 AY243002, Rh32 AY243003/

The skilled person can select an appropriate serotype, clade, clone or isolate of AAV for use in the present invention on the basis of their common general knowledge. It should be understood however that the invention also encompasses use of an AAV genome of other serotypes that may not yet have been identified or characterised.

The AAV genome used in the invention may be the full genome of a naturally occurring AAV virus. However, while such a vector may in principle be administered to patients, this will be done rarely in practice. The AAV genome may instead be derivatised for the purpose of administration to patients. Such derivatisation is standard in the art and the present invention encompasses the use of any suitable known derivative of an AAV genome, and derivatives which could be generated by applying techniques known in the art. Derivatisation of the AAV genome and of the AAV capsid (discussed below) are reviewed in Coura and Nardi (Virology Journal, 2007, 4:99), and in Choi et al. and Wu et al., referenced above.

Derivatives of an AAV genome include any truncated or modified forms of an AAV genome which allow for expression of a CDHR1 polypeptide from the vector in vivo in accordance with the present invention. Typically, it is possible to truncate the AAV genome significantly to include minimal viral sequence yet retain the above function. Reducing the size of the AAV genome in this way allows for increased flexibility in incorporating a CDHR1 transgene and other sequence elements such as regulatory elements within the vector. It may also reduce the possibility of integration of the vector into the host cell genome, reduce the risk of recombination of the vector with wild-type virus, and avoid the triggering of a cellular immune response to viral gene proteins in the target cell.

Typically, a derivative will include at least one inverted terminal repeat sequence (ITR), or two ITRs or more. Typically the vector will have two ITRs, that flank the transgene. In some cases, the ITRs may be derived from AAV genomes having different serotypes, or may be a chimeric or mutant ITR. An example mutant ITR is one having a deletion of a trs (terminal resolution site). This deletion allows for continued replication of the genome to generate a single-stranded genome which contains both coding and complementary sequences i.e. a self-complementary AAV genome. This allows for bypass of DNA replication in the target cell, and so enables accelerated transgene expression.

The one or more ITRs may flank a polynucleotide sequence encoding a transgene polypeptide (CDHR1) at either end. The inclusion of one or more ITRs may aid concatemer formation of the vector of the invention in the nucleus of a host cell, for example following the conversion of single-stranded vector DNA into double-stranded DNA by the action of host cell DNA polymerases. The formation of such episomal concatemers protects the vector construct during the life of the host cell, thereby allowing for prolonged expression of the transgene in vivo. The ITR sequences may, for example, be those of AAV2 having, for example, the sequence of SEQ ID NOs: 1 (5′ITR) and/or 9 (3′UTR) or variants having at least 80% or 85%, or 90%, or 95% or 98% or 99% sequence identity to SEQ ID NOs: 1 or 9, or up to 1, 2, 3, 4 or 5 insertions, deletions, or substitutions in the amino acid sequences of SEQ ID NO: 1 or 9.

In some embodiments, ITR elements may be the only AVV sequences retained in the vector. In some embodiments, one or more rep and/or cap genes or other viral sequences may be retained. Naturally occurring AAV virus integrates with a high frequency at a specific site on human chromosome 19, and shows a negligible frequency of random integration, such that retention of an integrative capacity in the vector may be tolerated in a therapeutic setting.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

### Capsid Coats, Viral Particles, Vesicles, Nanoparticles and Exosomes

A viral vector of the invention may have a capsid coat. Such an encapsidated vector may be referred to as a viral particle.

The vectors or particles of the invention include transcapsidated forms wherein an genome or derivative having the ITR(s) or other genome components of one serotype or virus type, for example AAV2, is packaged in the capsid of a different serotype, for example AAV8. This may be referred to as pseudotyping. The vectors or particles of the invention also include mosaic forms wherein a mixture of modified or unmodified capsid proteins from two or more different serotypes makes up the viral coat. The invention encompasses the provision of capsid protein sequences from different serotypes, clades, clones, or isolates within the same vector or viral particle. The vector may be a chimeric, shuffled or capsid modified derivative.

The capsid coat is typically selected to provide one or more desired functionalities for the viral vector, such as increased efficiency of gene delivery, decreased immunogenicity (humoral or cellular), an altered tropism range and/or improved targeting of a particular cell type compared to a viral vector comprising a naturally occurring genome. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

The capsid may determine the tissue specificity or tropism of a viral vector. Accordingly, the capsid serotypes for use in the invention will typically be one that has natural tropism for or a high efficiency of infection of the target cells. For example, AAV8 capsid serotypes have a natural tropism for cells of the retina, whilst AAV2 and AAV9 have a natural tropism for neurons. The vector may comprise an AAV8 capsid coat or a derivative thereof.

Chimeric capsid proteins include those generated by recombination between two or more capsid coding sequences of naturally occurring serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. For example, hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid genes created in this way by shuffling the capsid genes of several serotypes can be screened to identify viral clones having a desired functionality. Similarly, error prone PCR may be used to randomly mutate capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type. It may thereby confer improved binding to a target cell or improve targeting or the specificity of targeting of the vector to a particular target cell population, for example, photoreceptor cells of the retina. In other cases, the unrelated protein may be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al., referenced above. The vectors or particles also includes chemically modified forms bearing ligands adsorbed to the capsid surface. For example, such ligands may include antibodies for targeting a particular cell surface receptor.

In some cases, the viral or non-viral vectors described herein may be packaged in a vesicle, liposome, exosome or nanoparticle or other suitable means of packaging as are known to those skilled in the art.

### Retrovirus Derived Vectors

The vector may comprise a retrovirus genome or a derivative thereof. Derivatives of a retrovirus genome include any truncated or modified forms of a retrovirus genome which allow for expression of a CDHR1 transgene from the vector in vivo in accordance with the present invention.

As with AAV derived vectors, a retrovirus derived vector will typically comprise a derivative of a retroviral genome comprising the minimal viral sequences required for packaging and subsequent integration into a host. For retrovirus derived vectors, one or more long terminal repeats (LTRs) are the minimum element required for replication and packaging of the vectors and subsequent integration into the target cell to provide permanent transgene expression. However, other elements may also be present. For example, a human immuno deficiency virus (HIV) derived vector will typically comprises the HIV 5′ LTR, which is necessary for integration into the host cell genome, the Psi signal, which is necessary for packaging of viral RNA into virions, a promoter for the transgene, and the 3′ LTR. Other suitable retroviral vectors may for example be derived from murine leukemia virus (MuLV), gibbon ape leukemia virus (GaLV), Simian Immuno deficiency virus (SIV), and combinations thereof.

The tropism of a retrovirus derived vector is determined by the viral envelope proteins. Targeting of the appropriate cells, for example photoreceptor cells or RPE cells of the retina, may be enhanced by incorporating ligands for the target cells into the viral envelope.

### Adenovirus Derived Vector

The vector may comprise an adenovirus genome or a derivative thereof. Derivatives of an adenovirus genome include any truncated or modified forms of an adenovirus genome which allow for expression of a CDHR1 transgene from the vector in vivo in accordance with the present invention.

A large number of human adenoviral serotypes have been identified and they are categorized into six subgenera (A through F) based on nucleic acid comparisons, fibre protein characteristics, and biological properties. For example, group A includes serotypes 12 and 31, group B includes serotypes 3 and 7, group C includes serotypes 2 and 5, group D includes serotypes 8 and 30, group E includes serotype 4, and group F includes serotypes 40 and 41.

The core of an adenovirus virion contains the linear double-stranded DNA genome and associated proteins V, VII, X (mu), IVa2, and terminal protein (TP). The genome organization of different adenoviruses is conserved and has been proposed to have a timing function, wherein the ends of the genome are transcribed first (the immediate early genes E1 and E4 are located at opposite ends of the linear genome). Early transcription of E1 and E4 leads to the opening of the central region of the genome, allowing transcription of the central region.

Adenoviral genomes typically comprise eight RNA polymerase II transcriptional units: five early units, E1A, E1B, E2A-E2B, E3, and E4; two delayed early units, IX and IVa2; and the Major Late transcriptional unit. The Major Late transcriptional unit is further subdivided into L1-L5 regions based upon the use of alternative splicing sites. The transcriptional units often express proteins of similar function. For example, the E1A unit codes for two proteins responsible for activation of transcription and induction of S-phase upon cellular infection; the E1B transcription unit encodes two proteins that inhibit cellular apoptosis; the E3 transcriptional unit is involved in evasion of the immune response; and the Major Late transcriptional unit encodes structural proteins necessary for assembly of the capsid.

Heterologous transgene sequences may be inserted into adenoviral genomes, for example in the early transcriptional units and in the coding regions of various structural proteins, such as hexon, penton, and fiber. Deletions may have been made in the adenoviral genome (e.g., in the El regions) to create replication-defective adenoviral vectors, which have generally been considered safer for administration to human subjects.

In the present invention, the adenovirus may be any adenovirus or derivative suitable for delivery of the transgene to target cells. The adenovirus may be any serotype but is typically Ad5 or Ad2. An adenovirus derived vector of the invention may comprise all or part of the genome of any adenoviral serotype, as well as combinations thereof (i.e., hybrid genomes).

The adenoviral vector used in the invention may be either replication incompetent or replication competent. Such vectors are well known. For example, in a replication incompetent vector the E1 region may be deleted and replaced with an expression cassette with an exogenous promoter driving expression of the heterologous transgene. Usually, the E3 region is also deleted. Deletion of E3 allows for larger inserts into the E1 region. Such vectors may be propagated in appropriate cell lines such as HEK 293 cells which retain and express the E1A and E1B proteins. Other vectors also lack the E4 region, and some vectors further lack the E2 region. E2 and E4 vectors must be grown on cell lines that complement the E1, E4 and E2 deletions.

Vectors may also be helper dependent vectors, which lack most or all of the adenoviral genes but retain cis-acting sequences such as the inverted terminal repeats as well as packaging sequences that are required for the genome to be packaged and replicated. These vectors are propagated in the presence of a helper adenovirus, which must be eliminated from the vector stocks. Once again, such systems are well known in the art.

The capsid is composed of seven structural proteins: II (hexon), III (penton), IIIa, IV (fiber), VI, VII, and IX. The capsid comprises 252 capsomeres, of which 240 are hexon capsomeres and 12 are penton capsomeres. Hexon capsomeres, which are trimers of the hexon protein, make up about 75% of the protein of the capsid. Penton capsomeres, which are pentamers of the penton protein, are situated at each of the 12 vertices of the virion. Each penton capsomer is bound to six adjacent hexon capsomeres and a fiber. The fiber, which is usually a trimer of the fiber protein, projects from the penton capsomer. The hexon protein and, to a lesser extent, the fiber protein comprise the main antigenic determinants of an adenovirus and also determine serotype specificity.

An adenovirus derived vector is particularly suitable for use when a transient expression of a transgene is preferred.

### Herpes Simplex Virus Derived Vectors

The vector may comprise an herpes simplex virus (HSV) genome or a derivative thereof. Derivatives of an HSV genome include any truncated or modified forms of a HSV genome which allow for expression of a CDHR1 transgene from the vector in vivo in accordance with the present invention.

Herpes simplex virus (HSV) naturally establishes a life-long latent infection of human peripheral sensory neurons. Recombinant HSV vectors are genetically modified to be incapable of replication, but establish a latent-like state in neurons in vitro and in vivo.

### CDHR1

The term “CDHR1 polypeptide” as used herein encompasses any wildtype human CDHR1 as expressed by a healthy subject not having or expected to develop a CDHR1-associated retinal degeneration, for example CDHR1 having the amino acid sequence of SEQ ID NO: 6. The term also encompasses functional variants of such wildtype CDHR1, that is any variant that retains the normal function(s) of CDHR1 in vivo. CDHR1 appears to function in the development of nascent outer segment discs, possibly assisting in their horizontal elongation through connections with the periciliary ridge of the inner segment. There is then a cleavage event which likely severs the connections between CDHR1 and the inner segment binding partner (currently unidentified). Is has been proposed that ADAM10 may be this catalyst, although this has not been verified. ADAM10 is regulated by Sfrp1 (PMID: 32198470). Overall, CDHR1 functions in outer segment disc morphogenesis in order to produce regularly stacked outer segment discs. It is not known to have any other function. The normal function of a CDHR1 polypeptide could, for example, be tested in the Cdhr1−/− mouse model, for example with use of immunohistochemistry, western blot, ERG testing, or a combination of these tests.

In some cases, the CDHR1 polynucleotide may be a variant of the polynucleotide sequence of SEQ ID NO: 6 comprising one or more (for example up to 2, 3, 4, 5, 10, 15, 20, 25, 30, 35, 40 or 50) amino acid additions, substitutions or deletions. In some cases, the CDHR1 sequence may also have additional sequence elements or tags at the 5′ or 3′ end. Substitutions preferably introduce one or more conservative changes, which replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative change may introduce another amino acid that is aromatic or aliphatic in the place of a pre-existing aromatic or aliphatic amino acid. Conservative amino acid changes are well known in the art and may be selected in accordance with the properties of the 20 main amino acids as defined in Table 1 below.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

For the purpose of this invention, in order to determine the percent identity of two sequences (such as two polypeptide sequences), the sequences are aligned for optimal comparison purposes (e.g., gaps can be introduced in a first sequence for optimal alignment with a second sequence). The amino acids at each position are then compared. When a position in the first sequence is occupied by the same amino acid as the corresponding position in the second sequence, then the amino acids are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence, for example, for a CDHR1 polypeptide, SEQ ID NO: 6. If the sequence is shorter than SEQ ID NO: 1, the gaps or missing positions should be considered to be non-identical positions.

The skilled person is aware of different computer programs that are available to determine the homology or identity between two sequences using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. Other examples of suitable programs are the BESTFIT program provided by the UWGCG Package (for example used on its default settings) (Devereux et al (1984) Nucleic Acids Research 12, 387-395) and the PILEUP and BLAST algorithms c (for example used on its default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/).

Variants also include truncations, wherein a part of the sequence is deleted from the 5′ or 3′ end. Any truncation may be used so long as the variant is functional as described above. Truncations will typically be made to remove sequences that are non-essential for function in vivo and/or do not affect conformation of the folded protein, in particular folding of the active site or relevant binding site. Appropriate truncations can routinely be identified by systematic truncation of sequences of varying length from the N- or C-terminus.

Wildtype CDHR1 has 6 extracellular cadherin repeat domains, each of which contain calcium binding sites which appear crucial for its function (SEQ ID NOs: 13 to 18). The extracellular domains of CDHR1 are highly conserved which supports an important biological function. These may include binding sites for interacting partners (such as the inner segment binding partner, and the enzyme which cleaves the Cdhr1 ectodomain as shown by Rattner et al, 2004). The cytoplasmic domain is less conserved. Accordingly, in some cases the CDHR1 polypeptide used in the present invention comprises at least one, or at least 2, 3, 4, 5 or all 6 of the cadherin repeat domains corresponding to SEQ ID NOs: 13 to 18, or corresponding to SEQ ID NOs: 13 to 18 except for a small number of conservative changes as described above.

In some cases, the CDHR1 polypeptide is a codon-optimised version of the CDHR1 polypeptide of SEQ ID NO: 6, or of any other suitable CDHR1 polypeptide described herein.

A polynucleotide sequence encoding a CDHR1 polypeptide is any sequence which encodes such a CDHR1 polypeptide as described above. For example, vectors comprising a codon-optimised version of the nucleotide sequence of SEQ ID NO: 5 that encodes the same CDHR1 polypeptide of SEQ ID NO: 6 are explicitly encompassed within the scope of the present invention. In some cases the vector comprises the polynucleotide sequence of SEQ ID NO: 5.

### Promoters and Other Regulatory Elements

In the vector, the nucleic acid encoding the transgene product, i.e. the CDHR1 polypeptide, is typically operably linked to a promoter. In some cases the promoter may be constitutive i.e. operational in any host cell background, for example, the ubiquitous CAG promoter. More typically, the promoter is a cell-specific promoter, which drives expression a particular target cell type, for example photoreceptor cells of the retina. Examples of suitable promoters include the human rhodopsin kinase promoter (GRK1), which may have the sequence of SEQ ID NO: 2, or the human rhodopsin promoter, which may have the sequence of SEQ ID NO: 3, or functional variants thereof. One or more other regulatory elements, such as enhancers, postregulatory elements and polyadenylation sites may also be present in addition to the promoter. A regulatory sequence that is operably linked to the transgene is any sequences that facilitates or controls expression of the transgene, for example by promoting or otherwise regulating transcription, processing, nuclear export of mRNA or stability. The term “operably linked” means that the regulatory element is present at an appropriate position relative to another nucleic acid sequence (such as a transgene) so as to effect expression of that nucleic acid sequence., i.e. in their intended manner. A control sequence (e.g. a promoter) “operably linked” to a transgene is ligated in such a way that expression of the transgene is achieved under conditions compatible with the control sequences.

A vector of the invention may typically comprise the following elements in a 5′ to 3′ direction: (a) an inverted terminal repeat sequence (5′ITR), such as any ITR sequence or 5′ITR sequence described herein, or the sequence of SEQ ID NO: 1, or a variant having at least 70%, or 80% or 85% or 90% or 95% or 98% or 99% sequence identity to SEQ ID NO:1; (b) a promoter sequence, for example any promoter described herein, wherein the promoter is operably linked to a sequence encoding the CDHR1 polypeptide, for example the GRK1 promoter comprising the sequence of SEQ ID NO: 2; (c) a translation initiation sequence, such as the Kozak consensus sequence GCCACC; (d) optionally a chicken beta-actin promoter exon-intron-exon sequence (Ex/In/Ex), such as the sequence of SEQ ID NO: 4, or a variant having at least 70%, or 80% or 85% or 90% or 95% or 98% or 99% sequence identity to SEQ ID NO: 4; (e) a sequence encoding the CDHR1 polypeptide, such as the sequence of SEQ ID NO: 5; (f) optionally a woodchuck hepatitis post-transcriptional regulatory element (WPRE) having the sequence of SEQ ID NO: 7, or a variant having at least 70%, or 80% or 85% or 90% or 95% or 98% or 99% sequence identity to SEQ ID NO: 7; (g) a polyadenylation tail sequence, such as the bovine growth hormone polyadenylation tail sequence of SEQ ID NO: 8; and (h) a 3′ inverted terminal repeat sequence (3′ITR), such as any ITR sequence or 5′ITR sequence described herein, or the sequence of SEQ ID NO: 9, or a variant having at least 70%, or 80% or 85% or 90% or 95% or 98% or 99% sequence identity to SEQ ID NO:9. These components (a) to (h) ((d) and (f) being optional, i.e. each independently either present or absent) may be referred to as an expression cassette.

There may be intervening sequences between the some or all of the different components (a) to (h). An intervening sequence between any two adjacent elements in the sequence may in some cases be up to 200 nucleotides, or up to 150, 100, 75, 50, 40, 30, 20, 15, 10, or 5 nucleotides in length. The vector may also include additional nucleotide sequences encoding additional or alternative regulatory elements such as one or more (further) promoters or enhancers or locus control regions (LCRs). The vector may also comprise other sequence elements or remnants of sequence elements used for the construction, cloning, selection and so on of the vector, as are well known to those skilled in the art.

In some cases the vector comprises the sequence of any one of SEQ ID NOs: 10, 11 and 12. In some cases the vector has the sequence of SEQ ID NO: 10 or SEQ ID NO: 12, or variants thereof as set out below. In some cases the vector comprises the expression cassette (components (a) to (h)) within SEQ ID NOs: 10, 11 or 12, but a different vector backbone to that described in the Examples herein. For example, a different antibiotic resistance gene, such as kanamycin resistance gene, could be used in place of the ampicillin resistance gene. In some cases the vector comprises the expression cassette of any one of the vectors of SEQ ID NOs: 10, 11 and 12, except that one of the components (a) to (h) (or two or three or four of the components) is a variant of the component present in the reference sequence SEQ ID NOs: 10, 11 and 12 as set out above. For example, the vector may have the expression cassette of SEQ ID NO: 10, except that component (a) is a variant of the 5′ITR of SEQ ID NO: 1, having at least 70%, or 80% or 85% or 90% or 95% or 98% or 99% sequence identity to SEQ ID NO:1. In some cases the vector comprises a sequence having at least 70%, or at least 80%, or at least 85% or 90% or 95% or 98% or 99% or 99.6%, or 99.9% sequence identity to SEQ ID NO: 10, SEQ ID NO: 11 or SEQ ID NO: 12, optionally in combination with any of the sequence variation restraints set out above. For example, in one embodiment, the vector may comprise a sequencing having at least 95% sequence identity to the vector of SEQ ID NO: 10 or SEQ ID NO: 12, but each of components (a), (b), (c), (e), (g) and (h) is individually has at least 98% or 99% sequence identity to the corresponding component of SEQ ID NO: 10 or 12. In another example, the vector may have at least 95% sequence identity to the vector of SEQ ID NO: 10 or SEQ ID NO: 12, but comprise the full sequence of the expression cassette of SEQ ID NO: 10 or SEQ ID NO: 12, except that a component (a) is a variant of the 5′ITR of SEQ ID NO:1, having at least 70%, or 80% or 85% or 90% or 95% or 98% or 99% sequence identity to SEQ ID NO:1.

### Preparation of Vector

A vector of the invention may be prepared by standard means known in the art for provision of vectors for gene therapy. Thus, well established public domain transfection, packaging and purification methods can be used to prepare a suitable vector. This includes known methods for packaging vectors into vesicles, liposomes, exosomes or nanoparticles or the like.

Viral vectors used in gene therapy are typically generated by producing a cell line that packages a nucleic acid vector into a viral particle. The vectors typically contain the minimal viral sequences required for packaging and subsequent integration into a host, as described above, other viral sequences being deleted, leaving capacity for an expression cassette for one or more transgenes. The missing viral functions are typically supplied in trans by the packaging cell line.

Packaging cells are typically used to form virus particles that are capable of infecting a host cell. The packaging cells may be any suitable cell type known in the art. The packaging cells are typically human or human derived cells. Suitable cells include Human Embryonic Kidney (HEK) 293 or 293T cells, or HEK 293 derived cell clones (for example to package adenovirus derived vectors), Hela cells (for example to package HIV or other lentivirus derived vectors) and ψ2 cells or PA317 cells (for example to package retrovirus derived vectors). Other examples are BHK or CHO cells.

AAV derived vectors of the invention may comprise the full genome of a naturally occurring AAV virus in addition to the elements for gene therapy, i.e. a CDHR1 transgene. However, commonly a derivatised genome will be used, for instance a derivative which has at least one inverted terminal repeat sequence (ITR), but which may lack any AAV genes such as rep or cap.

In order to provide for assembly of a derivatised or recombinant genome into the viral particle, additional genetic constructs providing AAV and/or helper virus functions will be provided in a host cell in combination with the derivatised/recombinant genome. For AVV vectors, these additional constructs will typically contain genes encoding structural AAV capsid proteins i.e. cap, VP1, VP2, VP3, and genes encoding other functions required for the AAV life cycle, such as rep. The selection of structural capsid proteins provided on the additional construct will determine the serotype of the packaged viral vector. For replication incompetent viral vectors, helper virus functions, for example adenovirus helper functions, will typically also be provided on one or more additional constructs to allow for replication. The additional constructs may be provided as plasmids or other episomal elements in the host cell, or alternatively one or more constructs may be integrated into the genome of the host cell. Suitable genes and constructs may in some cases be any of those described herein.

The invention provides a host cell that produces the gene therapy vector as described herein. The host cell may have any suitable properties as described above.

The invention also provides a method for production of a vector of the invention. The method comprises providing a host cell according to the invention as described above and culturing the host cell under conditions suitable for the production of the vector. The method may comprise providing means and/or conditions for the replication of the vector and/or assembly of the vector into a viral particle and/or into other suitable packaging such as a vesicle, liposome, exosome or nanoparticle. Optionally, the method further comprises a step of purifying the vectors or viral particles and/or formulating the vectors or viral particles for therapeutic use.

The properties of the vectors and other products of the invention as described herein can be tested using techniques known by the person skilled in the art. In particular, a vector or other construct of the invention can be delivered to a test animal, such as a mouse, and the effects observed and compared to a control. Such use is also an aspect of the invention.

### Methods of Therapy and Medical Uses

The vectors described herein may be used as methods of treatment. Specifically, the vectors may be used as treatment for individuals with retinal degeneration attributed to biallelic variants in CDHR1. No current treatment exists for such patients, with inevitable blindness. Such methods of treatment form part of the present invention, as does use of the vectors and other products of the invention as described herein in the manufacture of a medicament for use in the treatments described herein. Specifically, the vectors may be used in the treatment, prevention or reversal of CDHR1-associated retinal degeneration or retinal dystrophy, or any condition associated with a loss of function of CDHR1 in a subject or patient. In some cases the subject may have biallelic null mutants of CDHR1. Examples are provided in Example 9 and Table 2. In other cases, the subject may have a one or more hypomorphic alleles of CDHR1. Conditions that may be treated include CDHR1-associated retinal degeneration or retinal dystrophy, cone-rod dystrophy, cone-dystrophy, rod-dystrophy, rod-cone dystrophy (retinitis pigmentosa), macular dystrophy, or late-onset macular dystrophy, macular degeneration, central areolar choroidal degeneration or geographic atrophy or any other retinal phenotype pathology attributed to sequence variants in the CDHR1 gene.

In some cases, the treatment achieves or is intended to achieve any one or more of the following effects:
Improvement from baseline in microperimetry
   Proportion of patients with improved microperimetry
Improvements in best corrected visual acuity (BCVA)
   ETDRS visual acuity chart
Improvements on retinal function on microperimetry
   Change in sensitivity (dB)
Spectral domain optical coherence tomography (SD-OCT)
   Changes in reflectivity of the ellipsoid zone
   Changes in outer segment length (i.e. regeneration of photoreceptor outer segments)
   Changes in outer nuclear layer thickness at pre-defined retinal loci (i.e. prevention of photoreceptor cell death)
   Changes in the external limiting membrane
Fundus autofluorescence (AF)
   To assess changes in the retina from baseline in autofluorescence imaging (i.e. reduction in the rate of central macular degeneration)
Visual Fields
   Octopus 900 pro will be used to assess changes in central peripheral vison from baseline
Electroretinography:
Changes in rod or cone responses
Changes in macular responses (i.e. pattern ERG)

Also, where applicable for patients: regeneration of the photoreceptor outer segment; prevention of photoreceptor (rod and/or cone) cell death; reduced rate of photoreceptor (rod and/or cone) cell death; increased photoreceptor layer thickness; increased retina thickness; increased superior retina thickness; increased inferior retina thickness; increased outer retinal thickness, increased inner retina thickness; increased distance between the outer plexiform layer and the retinal pigment layer; lengthening of the photoreceptor outer segment band; thickening of the ellipsoid band; increased distance between the inner and outer boundaries of the photoreceptors; thickening or regeneration of the external limiting membrane; restoration of the photoreceptor outer segment band; improved cone and/or rod photoreceptor function; improved/increased electroretinography (A-wave amplitudes and/or B-wave amplitudes) responses; improved eyesight or vision, improved eyesight or vision at low light intensity; improved night vision; a prevention of decline in any one or more of these measurements or the prevention of blindness. Any suitable method(s) may be used to measure these outcomes.

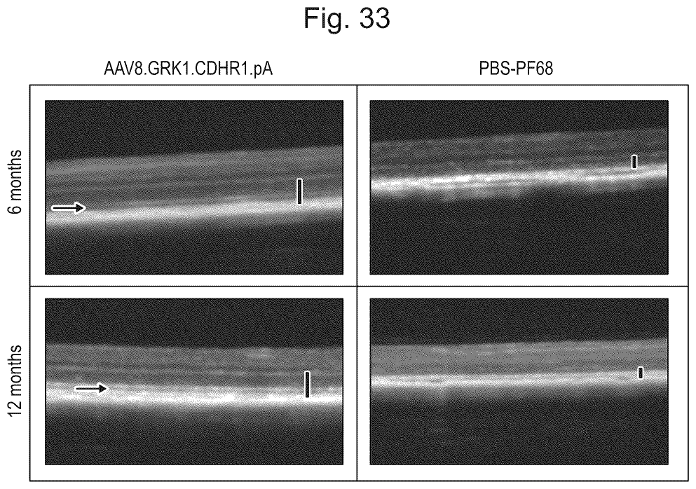

A particularly surprising result of the mouse model treatment described herein was the ability of the CDHR1-expressing vectors to reverse structural degeneration and the magnitude of functional responses seen at early timepoint in AAV-treated Cdhr1^{-/-} mice. For example, the improvement in A-wave responses following high-dose AAV injection was of a greater effect size than seen at the equivalent time-point in other pre-clinical proof-of-concept studies, such as Rpgr gene therapy (ref: PMID: 28549772). Moreover the beneficial effects appeared to increase over time as the degeneration progressed in untreated eyes. A sustained therapeutic effect is further supported by the surprising finding of preserved retinal thickness measurements in AAV-treated Cdhr1^{-/-} mice for 12 months following treatment. Detailed natural history studies using OCT imaging in Cdhr1 mice identified that relatively severe early functional deficits (affecting both rod and cones) was associated with poorly formed photoreceptor outer segments to which we attributed the early functional deficits; further functional losses were attributed to progressive photoreceptor cell death. Preservation of outer segments was therefore a key therapeutic goal following AAV gene therapy, since it is one of the key morphological characteristics of Cdhr1^{-/-} histological sections and human CDHR1-associated retinal degeneration. Importantly, restoration of the photoreceptor outer segments following AAV gene therapy was entirely unexpected and the most surprising of all demonstrated beneficial effects, since it implies restoration of the key function of Cdhr1—the morphogenesis of photoreceptor outer segments. These effects have not to our knowledge been shown following gene therapy in any pre-clinical model of retinal degeneration. Furthermore, the photoreceptor outer segments continued to regenerate between 1- and 6-months post-injection within the treated retina; both in terms of the reflectivity of the band representing outer segments, and in the extent of the regeneration. Moreover, the ellipsoid zone, formed of the junction of inner segment and outer segment boundaries was most hyperreflective at 12-months, with preservation of the regenerated outer segments (FIG. 33).

These observations are remarkable as they suggest sustained expression of Cdhr1 within episomes in photoreceptor cells, correct trafficking of Cdhr1 to the base of the photoreceptor outer segments, appropriate post-translational modification that permits Cdhr1 to interact with at least two as yet unconfirmed interacting molecules (the inner segment binding partner which permits horizontal outer segment disc elongation and the catalyst that cleaves its ectodomain, allowing outer segment discs to grow outwards towards the RPE). Furthermore, all of these beneficial molecular responses have been achieved through the expression of a cadherin cell surface molecule that may be considered an unattractive target for gene therapy given that Cdhr1 is a membrane-bound protein. However, at equivalent doses, we showed no toxicity in either wildtype or Cdhr1^{-/-} retinae.

The method of treatment may be regarded as a method of gene therapy. The term “gene therapy” means the therapeutic delivery of nucleic acid polymers into a subject, and usually to specific target cells, as discussed further below.

The subject may be a human or a non-human animal. Non-human animals include, but are not limited to, rodents (including mice and rats), and other common laboratory, domestic and agricultural animals, including rabbits, dogs, cats, horses, guinea pigs, cows, sheep, goats, pigs, chickens, amphibians, reptiles etc.

### Pharmaceutical Compositions and Modes of Administration

The one or more vectors or other therapeutic products of the invention as described herein may be formulated into pharmaceutical compositions. Such pharmaceutical compositions and their use in methods of treatment as described herein form part of the invention. Pharmaceutical compositions may comprise, in addition to the vector etc., a pharmaceutically acceptable excipient, carrier, buffer, stabiliser or other materials well known to those skilled in the art. Such materials should be non-toxic and should not interfere with the efficacy of the vectors. Examples of suitable compositions and methods of administration are provided in Esseku and Adeyeye (2011) and Van den Mooter G. (2006). The precise nature of the carrier or other material may be determined by the skilled person according to the route of administration. Examples of techniques and protocols can be found in Remington's Pharmaceutical Sciences, 20th Edition, 2000, pub. Lippincott, Williams & Wilkins.

The vectors of the invention may be administered by any suitable route and means that allows for transduction of the target cells. The target cells are rod and cone photoreceptor cells within the retina. Typically, delivery is by subretinal injection, or less commonly, by intravitreal injection. The vector may be delivered surgically beneath the retina, for example by sub-retinal injection.

For injection at the site of affliction, the active ingredient will be in the form of an aqueous solution which is pyrogen-free and has suitable pH, isotonicity and stability. Those of relevant skill in the art are well able to prepare suitable solutions using, for example, isotonic vehicles such as phosphate-buffered saline, Sodium Chloride Injection, Ringer's Injection, Lactated Ringer's Injection, Hartmann's solution. Preservatives, stabilisers, buffers, antioxidants and/or other additives may be included, as required.

Dosages and dosage regimes can be determined within the normal skill of the medical practitioner responsible for administration of the composition. The dose of a vector of the invention may be determined according to various parameters, especially according to the age, weight and condition of the patient to be treated; the route of administration; and the required regimen.

Administration is typically in a “prophylactically effective amount” or a “therapeutically effective amount” (as the case may be, although prophylaxis may be considered therapy), this being sufficient to show benefit to the individual. For example, a therapeutically effective amount of a vector of the invention or an effective method of treatment in accordance with invention may be one that results in expression of the transgene in target cells/photoreceptor cells. Outcome measures are as described elsewhere herein above.

In other words, the treatment is sufficient to result in a clinical response or to show clinical benefit to the individual, for example to cure disease, prevent or delay onset or progression of the disease or condition or one or more symptoms, to ameliorate or alleviate one or more symptoms, to induce or prolong remission, or to delay relapse or recurrence. In some cases the treatment is sufficient to improve the subject's eyesight. In some cases the treatment is sufficient to slow down, reduce or prevent (further) degeneration of the subject's sight over time. More specifically the treatment may in some cases improve or reduce loss of vision in low light conditions.

A typical single dose of the one or more vectors of the invention may between 10⁸, or 10⁹ or 2.5×10⁹ or 5×10⁹; and 10¹⁵, or 10¹⁴, or 10¹³, or 10¹², 10¹¹ or 5×10¹⁰ or 2.5×10¹⁰ or 10¹⁰ viral genomes (vg), or any range thereof, such as 2.5×10⁹ to 5×10¹⁰ vg. A dose at the lower end of these ranges will typically be used for administration direct to the retina, whilst a dose at the higher end of the range will typically be needed for systemic administration.

The dosing range of vectors used for retinal gene therapy in patients is determined through phases 1-3 of clinical trial. However, extensive dosing studies of vectors expressing the CDHR1 transgene were performed in Cdhr1−/− and C57BL6J (wildtype mice). These data suggest a therapeutic dosing range, for example for GRK1.CDHR1.pA in the Cdhr1−/− mouse. In the case of CDHR1, the finding of therapeutic efficacy at a dose of 1.5×10⁸ is a log unit less than was required to show a therapeutic benefit in a mouse model of RPGR-related retinitis pigmentosa (PMID: 28549772). A dose of 2×10⁹ of AAV8.RPGR was required to show therapeutic benefit in the Rpgr mouse model. Benefit was shown in phase 1 clinical trials with the resulting vector (PMID: 32094925) with doses ranging from 5×10¹⁰-2.5×10¹¹. An approximation suggests that a dosing range of 5×10⁹-2.5×10¹⁰ may be sufficient to show a therapeutic benefit in patients with CDHR1-associated retinal degeneration.

A single AAV capsid that contains a single stranded DNA molecule is a single viral genome (vg). Vg can be quantified by any suitable method as well known in the art, for example real-time PCR. The one or more vectors are preferably administered only once, resulting, depending on the vector used, in permanent or transient knock down of the target gene, but repeat administrations, for example in future years and/or with different serotypes may be considered.

A composition of the invention may be administered or for administration alone or in combination with other suitable therapeutic compositions or treatments. Published data exist regarding the safety of adjunctive substances, such as blue dye to aid subretinal delivery (PMID: 28706756), or hydroxychloroquine to augment the efficacy of gene therapy (PMID: 31309129)

### Kits

The vectors, pharmaceutical compositions or other products of the invention as described herein can be packaged into a kit. The kit may additionally comprise suitable means for administering the product and/or instructions for use. Such kits are an aspect of the present invention.

### EXAMPLES

### Example 1—Deep Phenotyping of the Cdhr1^{-/-} Mouse

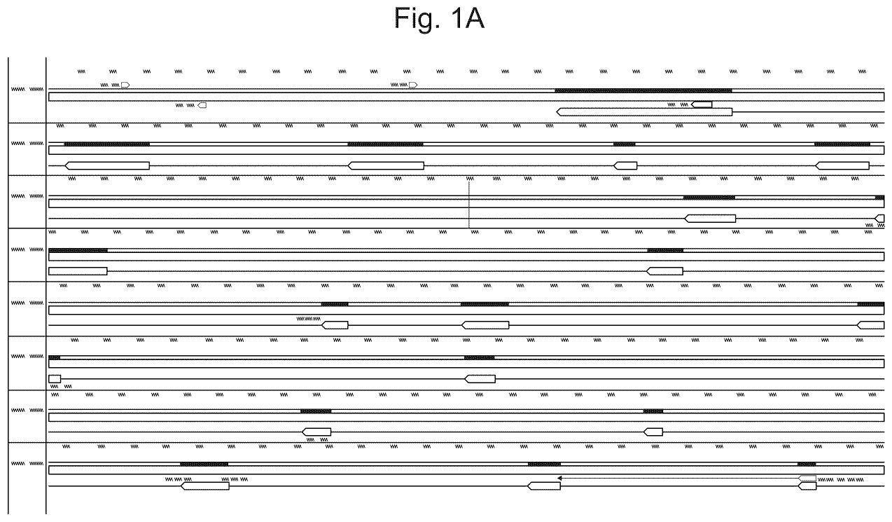

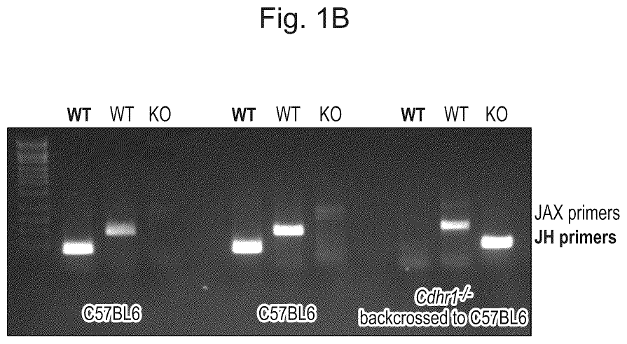

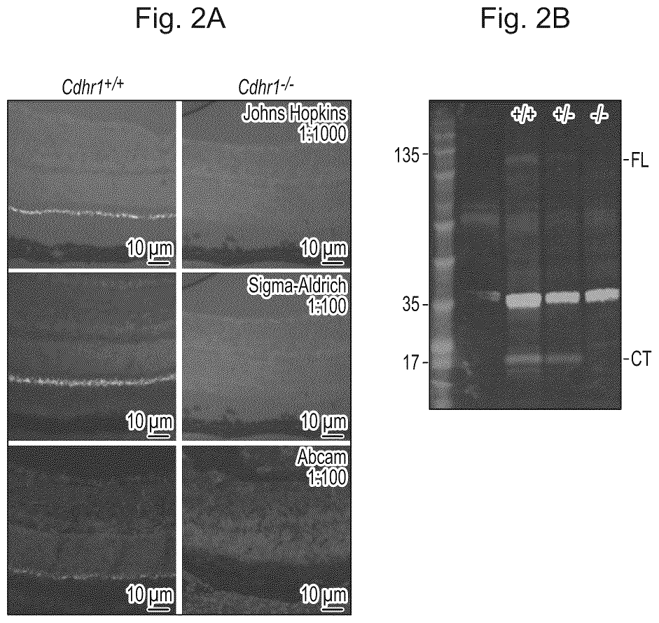

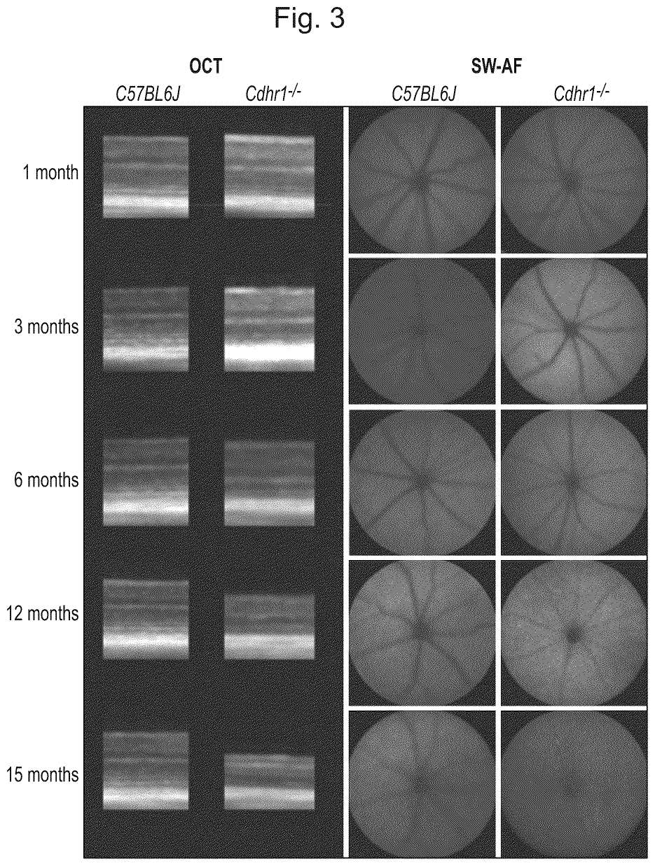

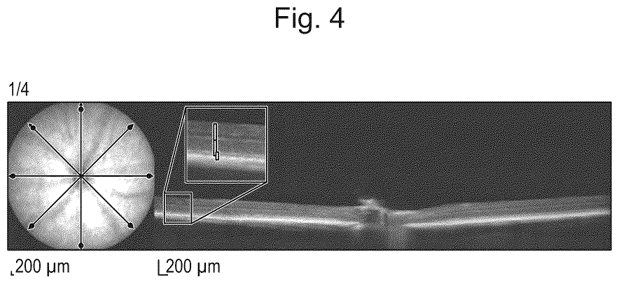

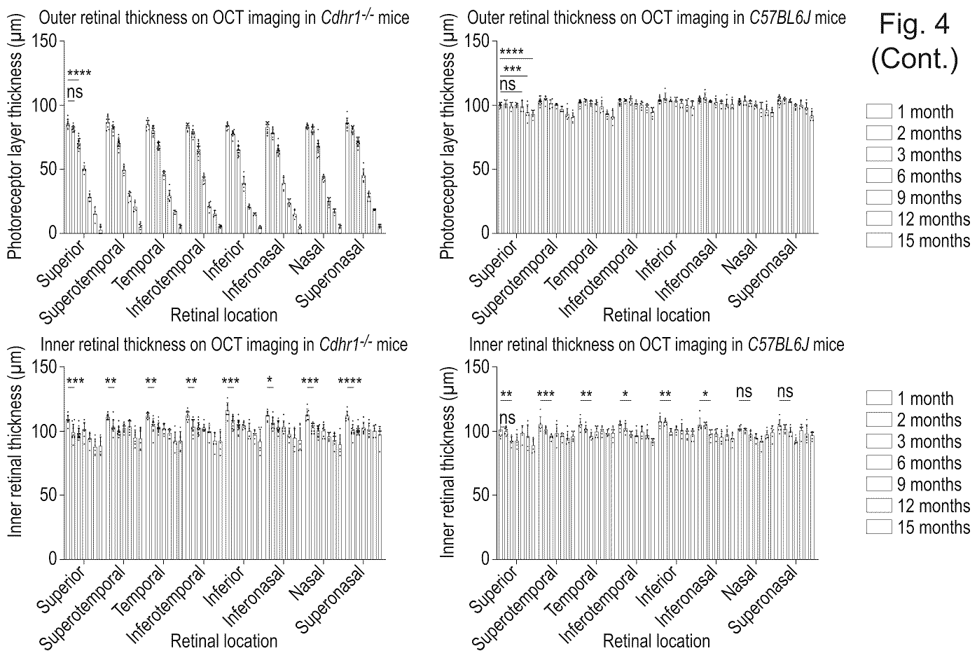

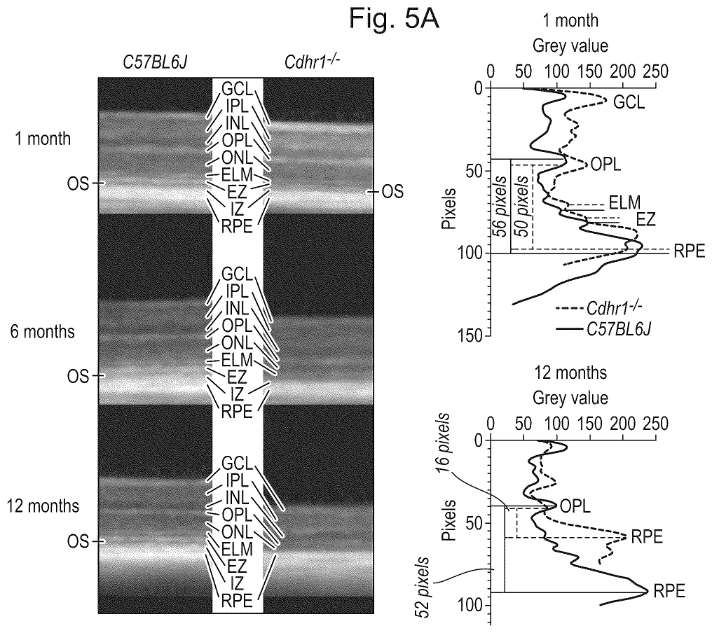

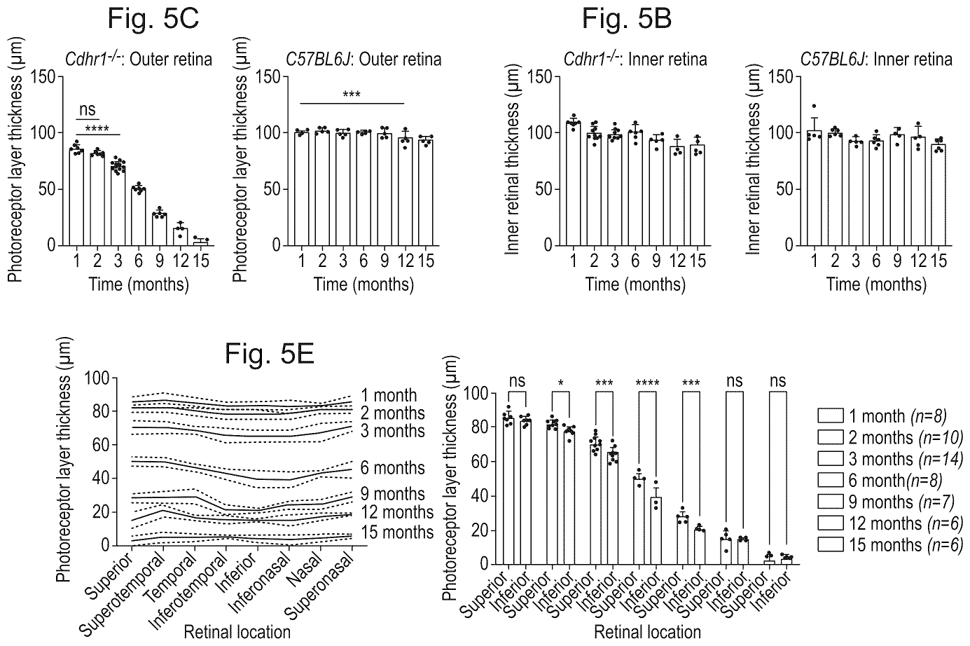

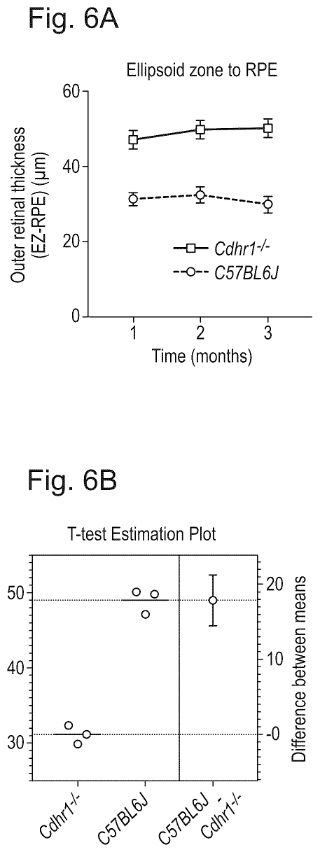

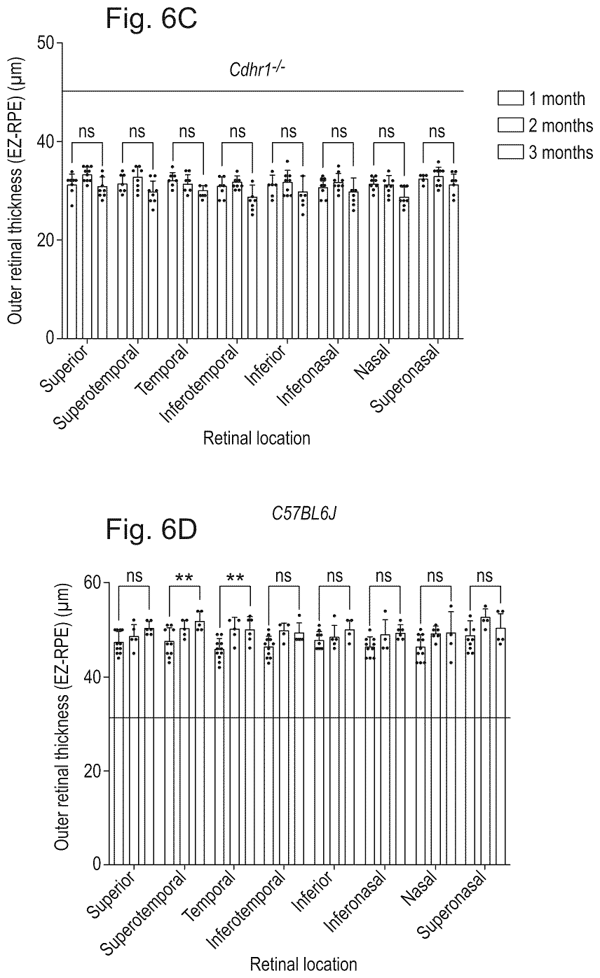

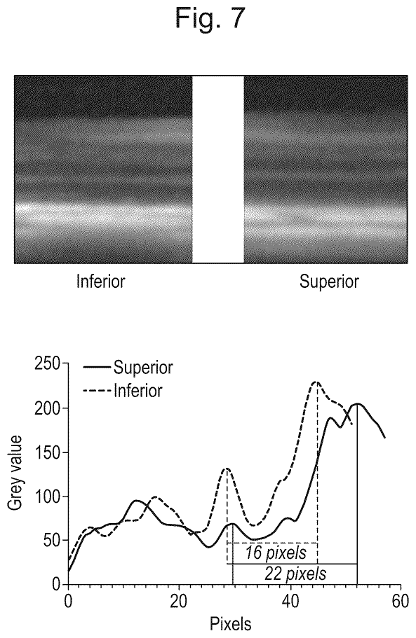

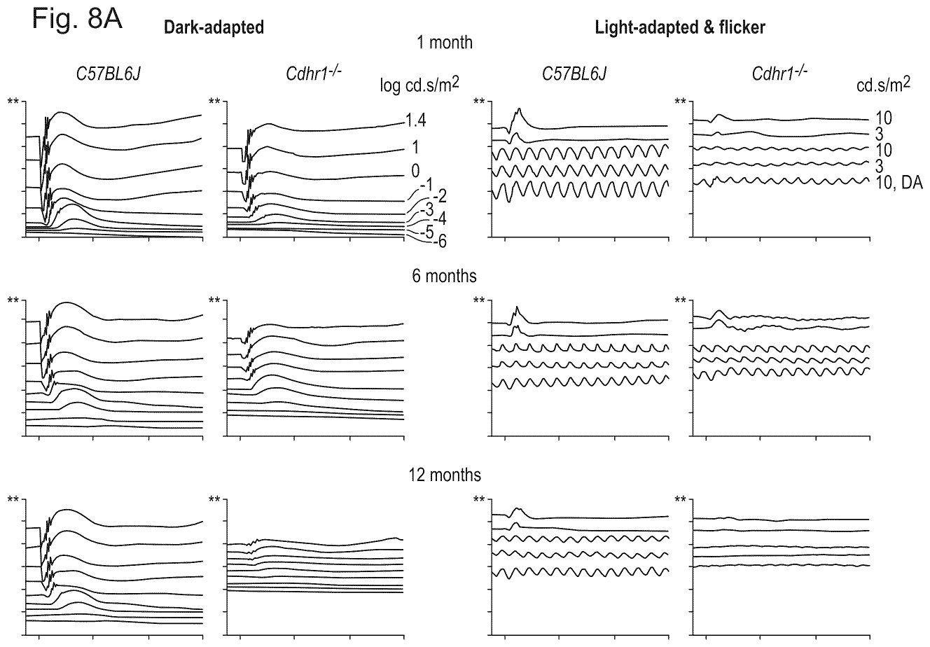

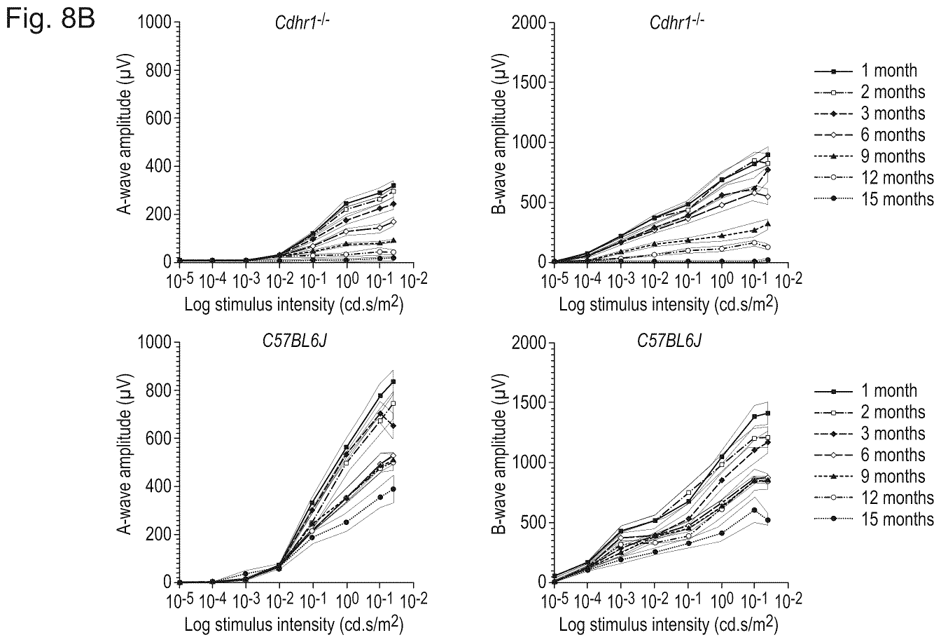

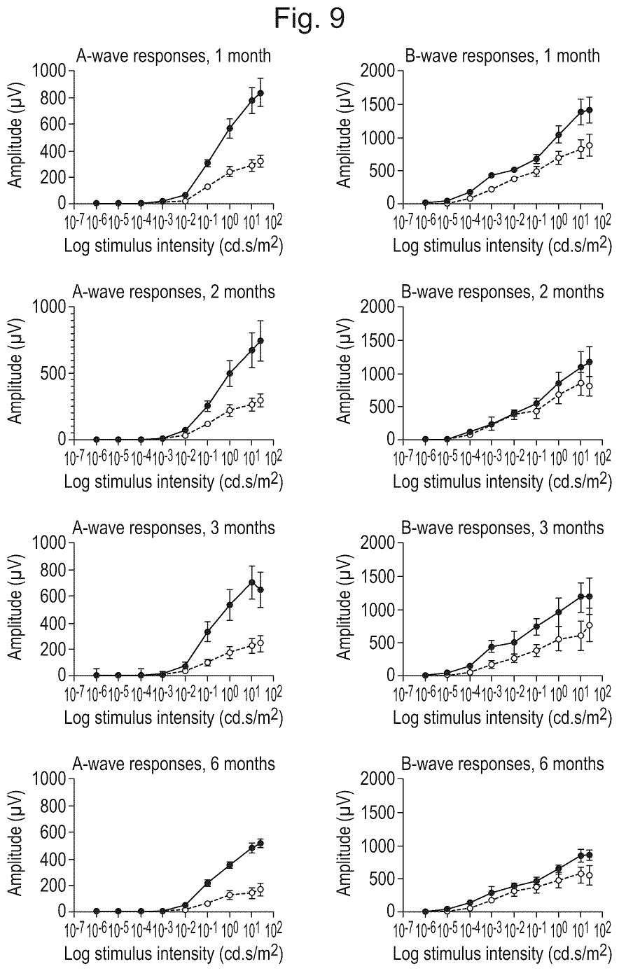

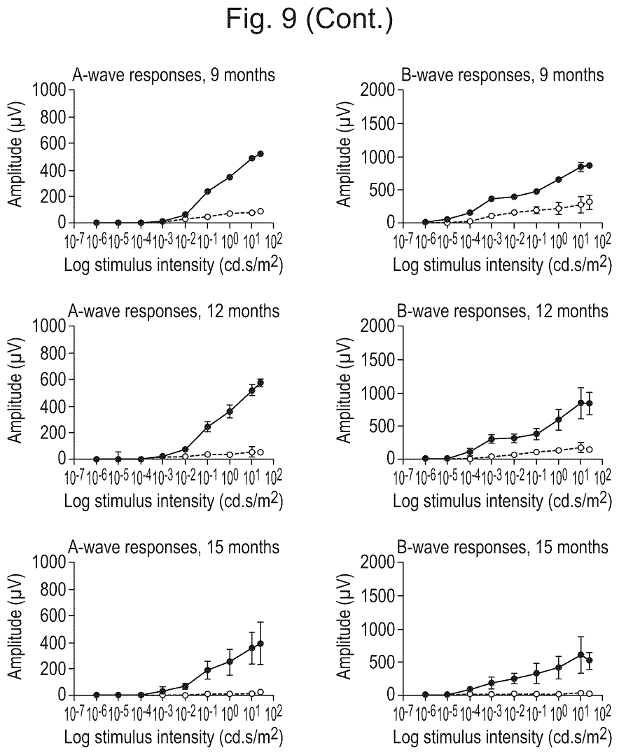

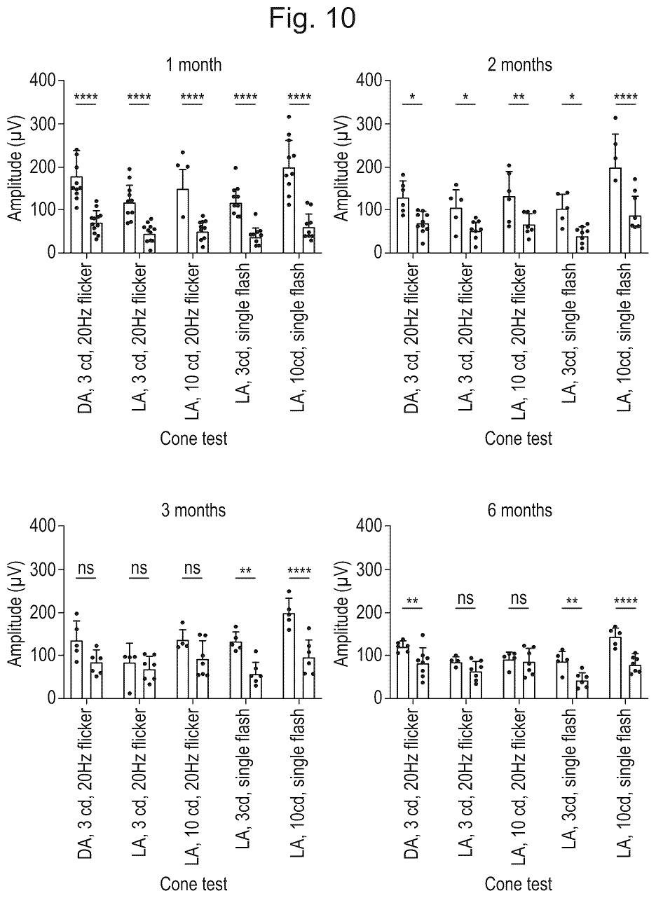

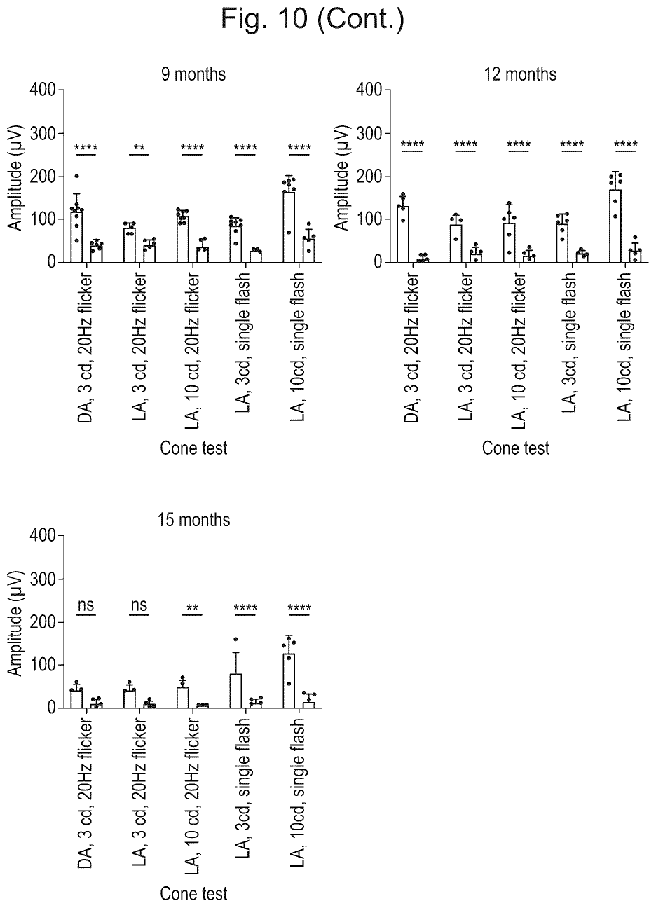

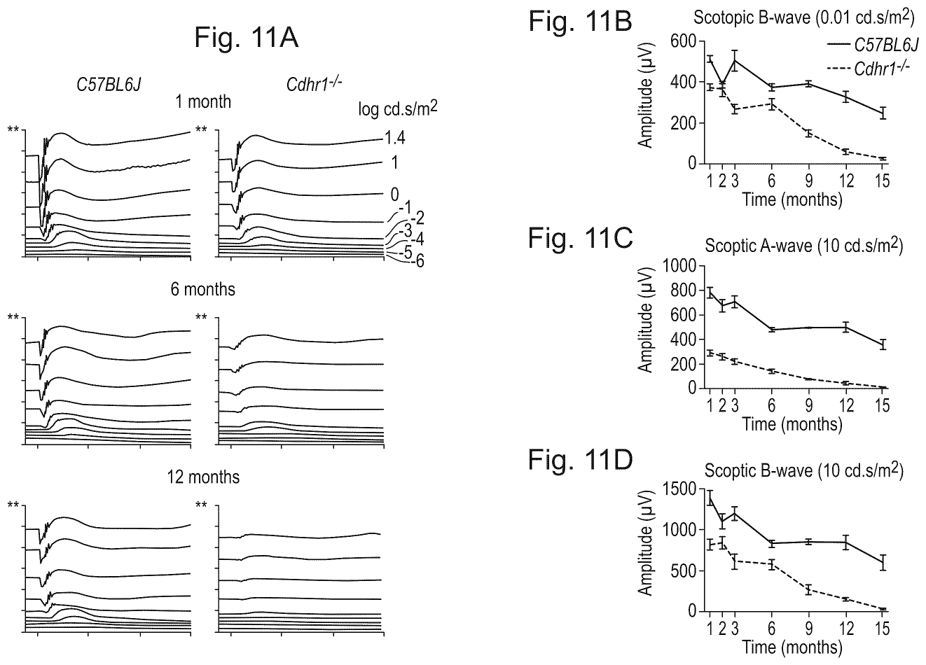

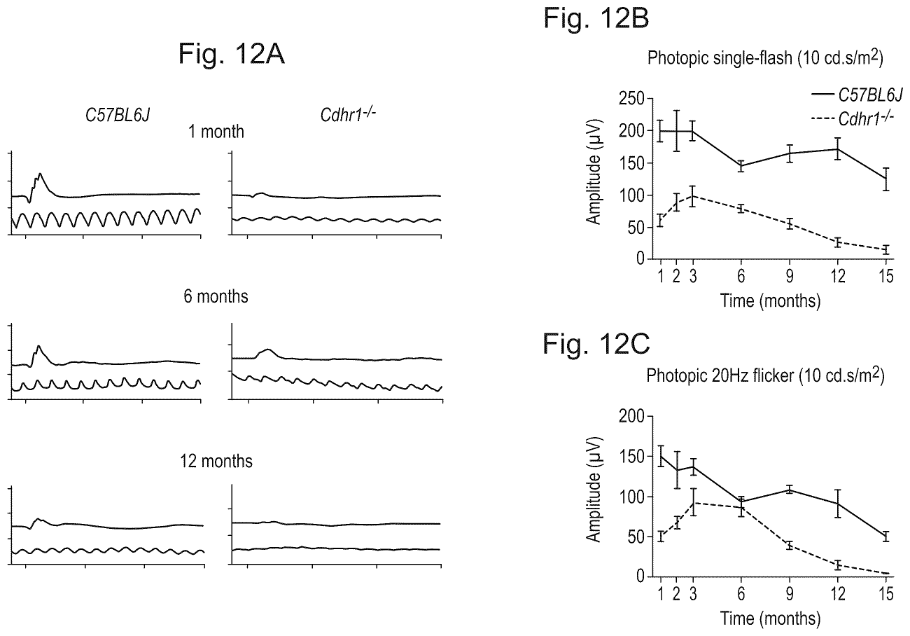

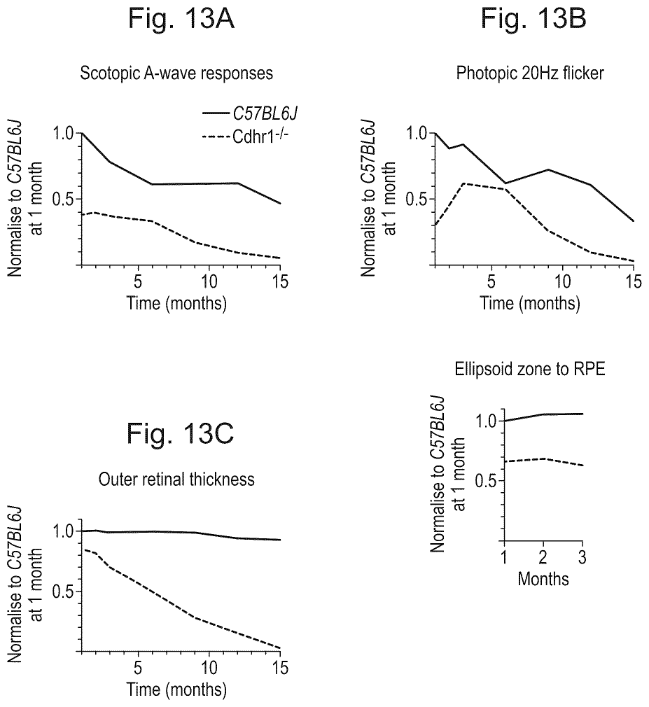

Retinal imaging studies of Cdhr1 mutant mice have never previously been undertaken. The objectives of retinal imaging studies in Cdhr1 mutant mice and experimental results pertaining to that objective are presented below.
(1) To accurately genotype Cdhr1^{-/-} mice: New primer pairs were designed that enabled accurate genotyping of Cdhr1^{-/-} litters when backcrossed onto a C57BL6J background (FIG. 1). Confirmation of immunohistochemical and biochemical absence of Cdhr1 in mice is presented in FIG. 2. The aim of immunohistochemistry experiments was to identify and localise Cdhr1 in Cdhr1^{+/+} mice, and demonstrate the absence of Cdhr1 protein in Cdhr1^{-/-} mice. In addition, immunohistochemistry using validated antibodies will later be used in Cdhr1^{-/-} mice following gene rescue with AAV8.CDHR1 to identify human Cdhr1 protein expression. Four antibodies against human Cdhr1 were tested, with varied antigenic epitopes across the Cdhr1 protein (Abcam Polyclonal Rabbit 98% homology Amino acid 50-100; Sigma-Aldrich Polyclonal Rabbit 84% homology Amino acid 250-425; Santa-Cruz Monoclonal Rabbit 92% homology Amino acid 327-529; and Johns Hopkins Polyclonal Rabbit 87% homology Amino acid 844-859). Three antibodies stained retinas of Cdhr1^{+/+} mice at the junction of inner and outer segments of photoceptors in a pattern identical to that described previously with the Johns Hopkins antibody only.^{1, 7} The Santa-Cruz antibody did not detect Cdhr1 protein at any concentration (data not shown). None of the three antibodies stained Cdhr1^{-/-} retinas at the same primary antibody concentrations. Given the distribution of target epitopes and the absence of a signal with all three primary antibodies, it is unlikely that the Cdhr1^{-/-} mice produce a truncated form of Cdhr1.
(2) To determine which measures of retinal thickness allow distinction between Cdhr1^{-/-} and WT mice on SD-OCT: Spectral-domain optical coherence tomography (SD-OCT) imaging allows visualisation of the retinal layers in vivo, permitting longitudinal assessment. Cdhr1 is expressed at the base of the outer segments and candidate OCT measures of retinal thickness focused on this region. The ellipsoid zone is lost entirely by 6 months of age in Cdhr1^{-/-} mice which limits its utility for longitudinal assessment. (FIG. 3) The external limiting membrane (ELM) was preserved to 6 months and interdigitation zone and retinal pigment epithelium (RPE) to 12 months of age in Cdhr1^{-/-} mice. Therefore, outer plexiform layer (OPL) to RPE was used as a primary outcome measure. These measures identified a statistically significant difference between Cdhr1^{+/+/+/-} and Cdhr1^{-/-} mice at all timepoints from 1-month of age (FIG. 4).
(3) To determine the rate of retinal degeneration in Cdhr1^{-/-} mice. Previous histological data suggested a 50% reduction in outer nuclear layer thickness in Cdhr1^{-/-} mice compared to WT controls at 6 months.¹ In vivo imaging data with SD-OCT shows a slowly progressive rate of outer retinal degeneration over time which appears consistent with earlier histological studies (FIG. 4) There was no evidence of an inner retinal degeneration when compared to wildtype (FIGS. 4 and 5). Cdhr1^{-/-} mice were found to exhibit inferior retinal degeneration, suggesting a possible modifier role of light in the degeneration (FIGS. 5 and 7).
(4) To determine whether Cdhr1^{-/-} mice demonstrate qualitative abnormalities on retinal imaging studies. Short-wavelength AF imaging in Cdhr1^{-/-} mice demonstrates punctate autofluorescent signal as early as 4 weeks of age in some mice, persisting through 12 months (the latest time point tested) shown in. Autofluorescent dots vary in position over time, vary between eyes and between Cdhr1^{-/-} animals. They are rarely seen in Cdhr1^{+/+} or Cdhr1^{+/-} mice, and when present, often only at advanced age. Hyperautofluorescent dots have been demonstrated in mouse models of other retinal disorders and are thought to represent sub-retinal macrophage response to retinal degeneration.⁶ This feature helps to distinguish Cdhr1^{-/-} mice from other genotypes in combination with SD-OCT imaging and PCR analysis to reliably genotype littermates. OCT imaging further identified key structural features supporting histological descriptions. Cdhr1^{-/-} mice lack formed photoreceptor outer segments which is reflected by a shortened photoreceptor outer segment on OCT at the earliest timepoint tested (FIG. 5). Reflectivity profiles of OCT images confirm this. Longitudinal retinal thickness measurements on OCT show that the layers in the outermost retina (EZ-RPE) do not change in Cdhr1^{-/-} mice over the 3 months in which they are measurable (after which the ellipsoid zone is lost). These segments are shortened compared to wildtype (FIG. 6). Since photoreceptor outer segments are essential for phototransduction, it is hypothesized that the significant functional deficits seen in Cdhr1^{-/-} mice are due to morphological abnormalities in the photoreceptor outer segments, and that functional losses over time are due to progressive photoreceptor cell death.
(5) To determine the natural history of rod and cone dysfunction in Cdhr1^{-/-} mice. Dark- and light-adapted electroretinography recordings (FIGS. 8 and 9) show a significant deficit in rod and cone function in Cdhr1^{-/-} mice from 1-month of age (˜60% reduction compared to wildtype (FIGS. 11-13) which is approximately the proportion of thinning of the outermost retinal layers which include the outer segments), followed by progressive decline in rod function. Cone responses appear to improve to 3-months, followed by progressive decline with increasing age.

### Example 2—Design of AAV8.CDHR1 Vectors and In Vivo Transgene Expression Following Subretinal Injection

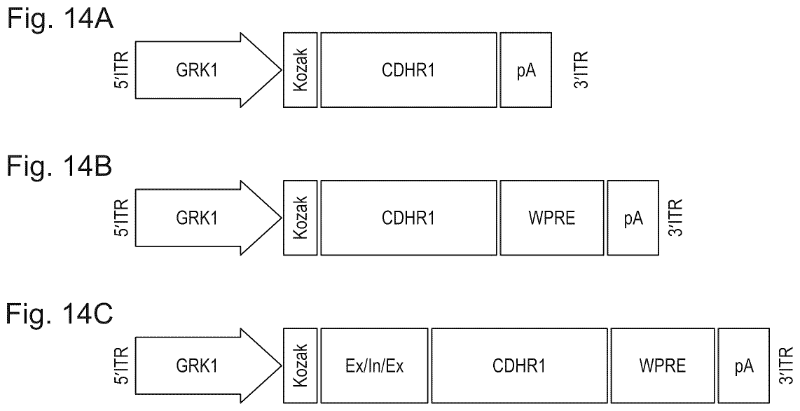

The full-length human CDHR1 coding sequence (NCBI transcript ID: NM_033100; 2,580 bp) was amplified by KOD polymerase chain reaction, and subcloned into a vector backbone containing the human rhodopsin kinase promoter (GRK1, 199 bp), a Kozak consensus sequence (GCCACC) for translation initiation in the 5′ position, and a downstream bovine growth hormone polyadenylation tail for the stabilisation of mRNA transcripts (GRK1.CDHR1.pA). A second plasmid construct was created in which cis-acting enhancing sequences were added; the exon-intron-exon sequence from the chicken beta-actin promoter cloned in following the GRKI promoter and the woodchuck hepatitis post-transcriptional regulatory element (WPRE) downstream of the CDHR1 coding sequence (GRK1.In.CDHR1.WPRE.pA). A third plasmid construct omitted the exon-intron-exon sequence, but the unenhanced (A), and the maximally enhanced (C) constructs were packaged into AAV for downstream assessments. A schematic representation of the three vector construct designs containing the human CDHR1 transgene are shown in FIG. 14.

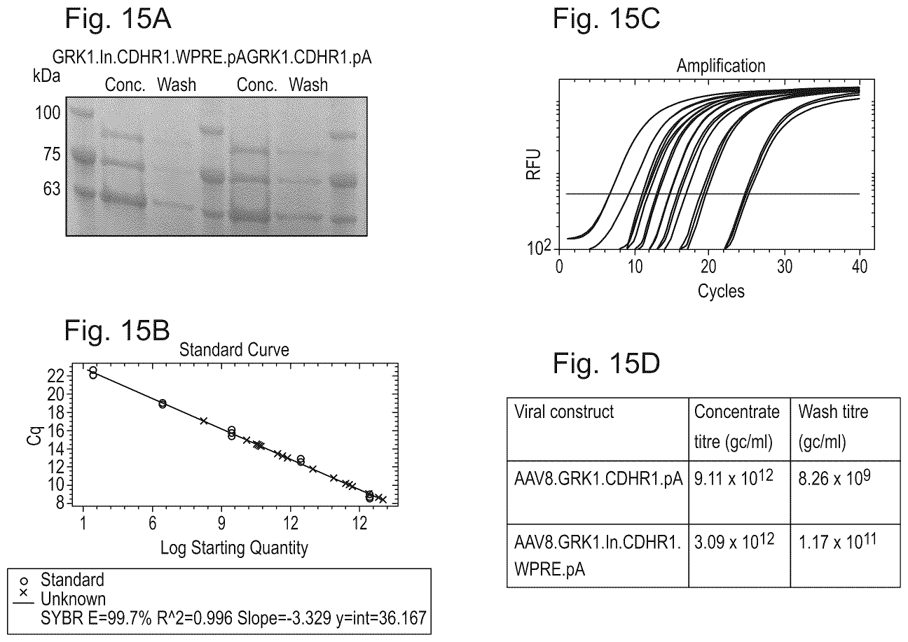

The CDHR1 expressing constructs were sequence verified, amplified and purified. Two of the three constructs, were selected for production of experimental AAVs (p.AAV8.GRK1.CDHR1.pA and p.AAV8.GRK1.In.CDHR1.WPRE.pA), being packaged into wildtype AAV8 capsids (PlasmidFactory, Bielefield, Germany) following culture in HEK293T cells, lysis and isolation of viral particles using an iodixanol gradient, and purification. SDS-polyacrylamide gel electrophoresis confirmed viral purity through the presence of AAV viral capsid proteins (VPN1, VPN2, VPN3) without contaminants (FIG. 15). AAV titre was achieved using quantitative polymerase chain reaction (qPCR) with primers directed against the CDHR1 transgene; achieved viral titres were 8.95×10¹² for GRK1.CDHR1.pA and 2.97×10¹² for GRK1.In.CDHR1.WPRE.pA (FIG. 15). Dilution of AAV was performed on the day of injection from AAV stocks using sterile PBS diluted with Pluronic® F-68 to a final concentration of 0.001%.¹¹

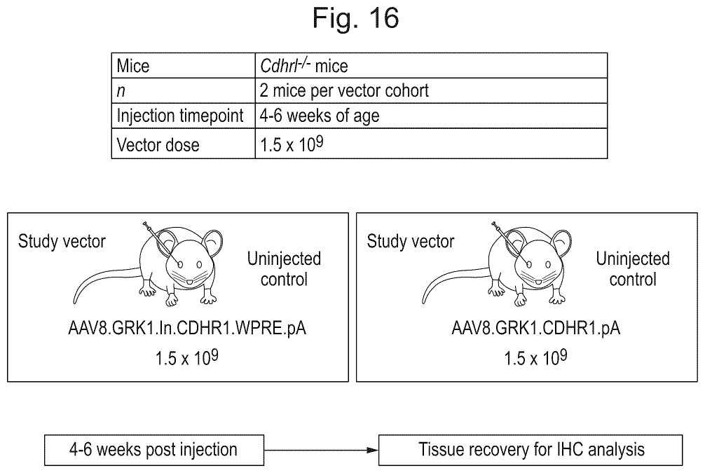

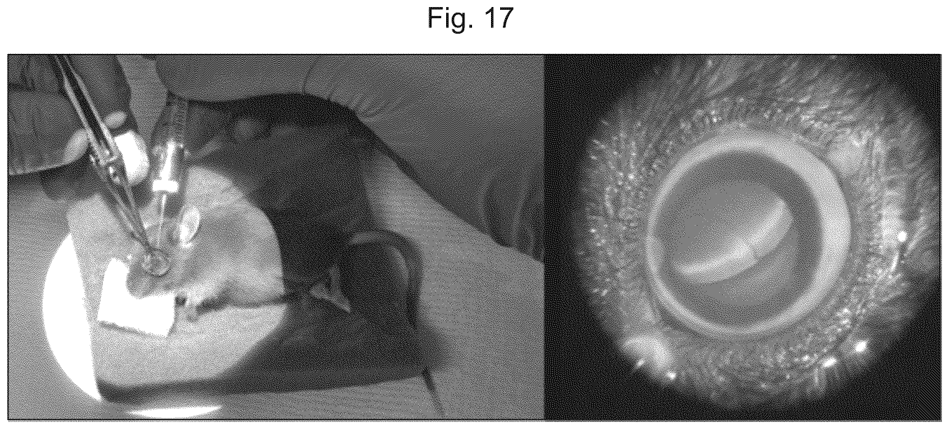

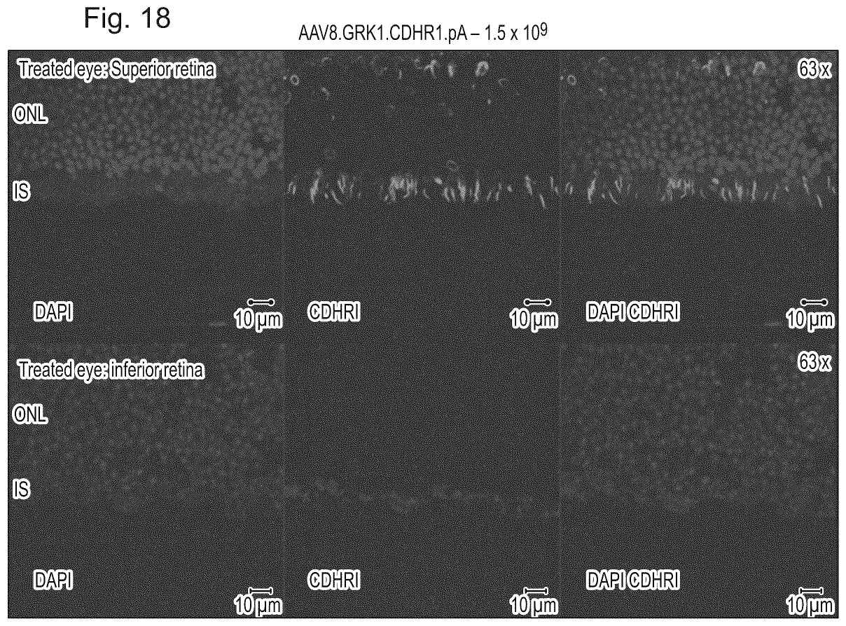

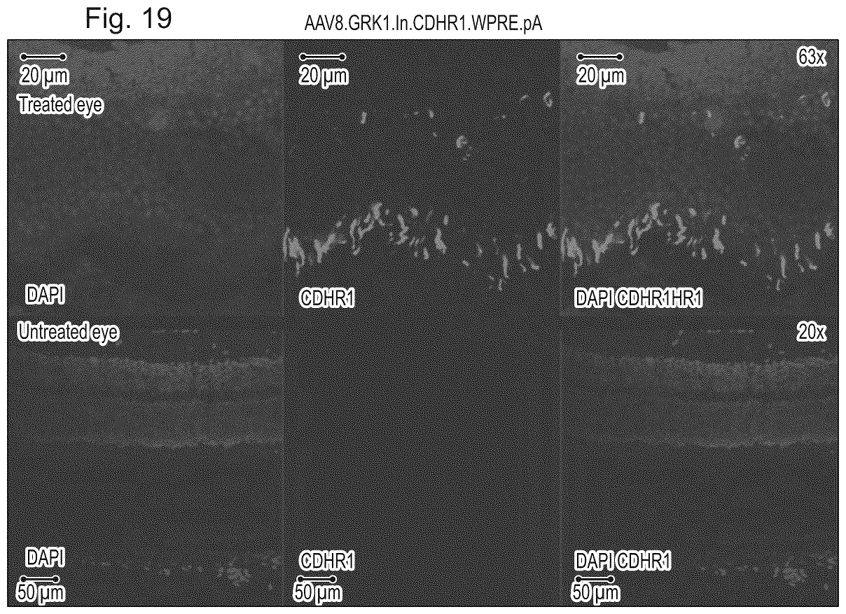

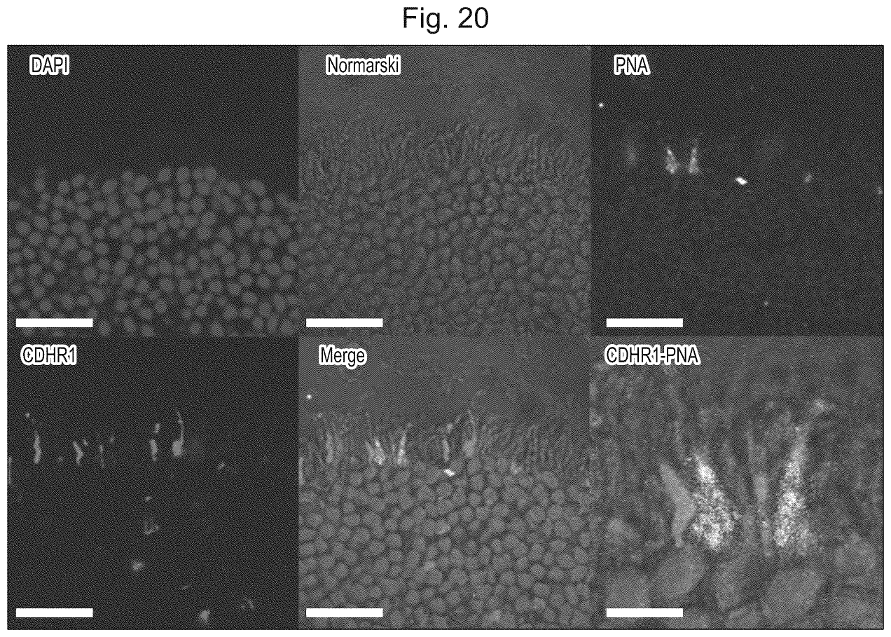

In the absence of a stable photoreceptor cell line, expression of CDHR1 was demonstrated in vivo in the Cdhr1^{-/-} knockout mouse line. The AAV8.GRK1.CDHR1.pA and AAV8.GRK1.In.CDHR1.WPRE.pA vectors were delivered by subretinal injection at a dose of 1.5×10⁹ with vehicle control (PBS-0.001% PF68) of the same volume delivered to the fellow eye to control for the effect of surgical retinal detachment (experimental design summarised in FIG. 16 with surgical techniques shown in FIG. 17). Paired eyes were harvested, fixed, cryosectioned and immunostained with antibodies against CDHR1. The superior retinas of AAV8.CDHR1 treated eyes exhibited immunostaining of CDHR1 which localised to the inner segment with perinuclear staining evident in the outer nuclear layer (FIGS. 18 and 19). This finding was evident using anti-CDHR1 antibodies directed against the N-terminal cadherin repeats, and the extreme c-terminus suggesting expression of the full-length protein (data not shown). The inferior retinas of treated eyes exhibited a more diffuse distribution of CDHR1 within the inner segments (FIG. 18). PBS-injected control eyes did not express CDHR1 (FIG. 19). Sub-retinal delivery of both vectors at a dose of 1.5×10⁹ constituted an overexpression of CDHR1 on immunohistochemistry in comparison to expression in C57BL6J mice (FIG. 2). Expression of CDHR1 in cone photoreceptors was confirmed using peanut agglutinin (PNA) which stains cone photoreceptor sheaths; following AAV8.CDHR1 delivery, CDHR1 co-localised with PNA indicating expression of CDHR1 in cone photoreceptors (FIG. 20).

### Example 3—AAV8.CDHR1 Vector Dosing Study

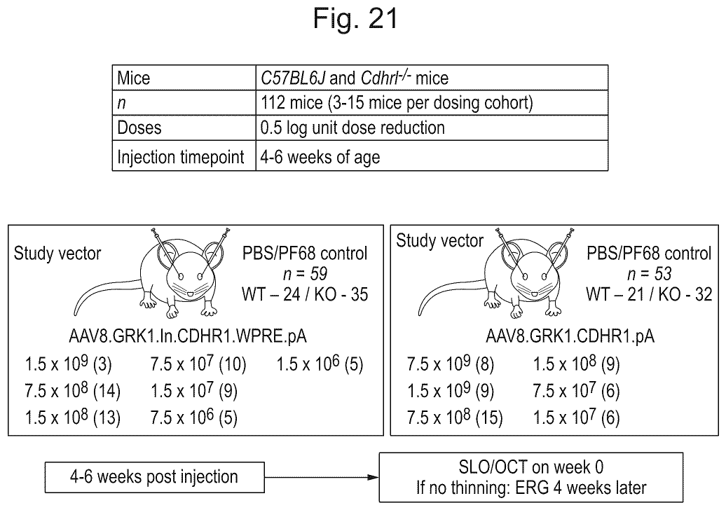

The maximum tolerated sub-retinal dose of each AAV8.CDHR1 vector was determined using outer retinal thickness measurements on optical coherence tomography (OCT) imaging 4-weeks after intervention (experimental design is summarised in FIG. 21). An assessment was made in both Cdhr1^{-/-} and C57BL6J mice, in order to determine the safe dosing limit across both groups. A dose-reduction strategy was used whereby 0.5 log unit decrements in dose from 1.5×10⁹ gc were assessed for each AAV8.CDHR1 vector. In order to control for the effect of retinal detachment on retinal thickness measurements, paired control eyes were injected with an equivalent volume of vehicle control (PBS-PF68 0.001%).

### AAV8.GRK1.CDHR1.pA Vector Dosing

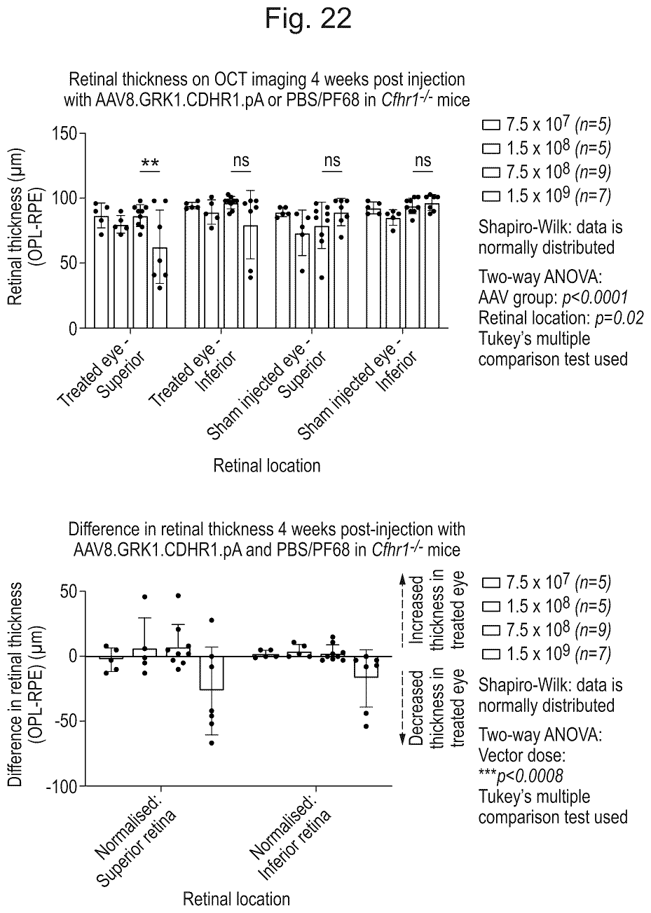

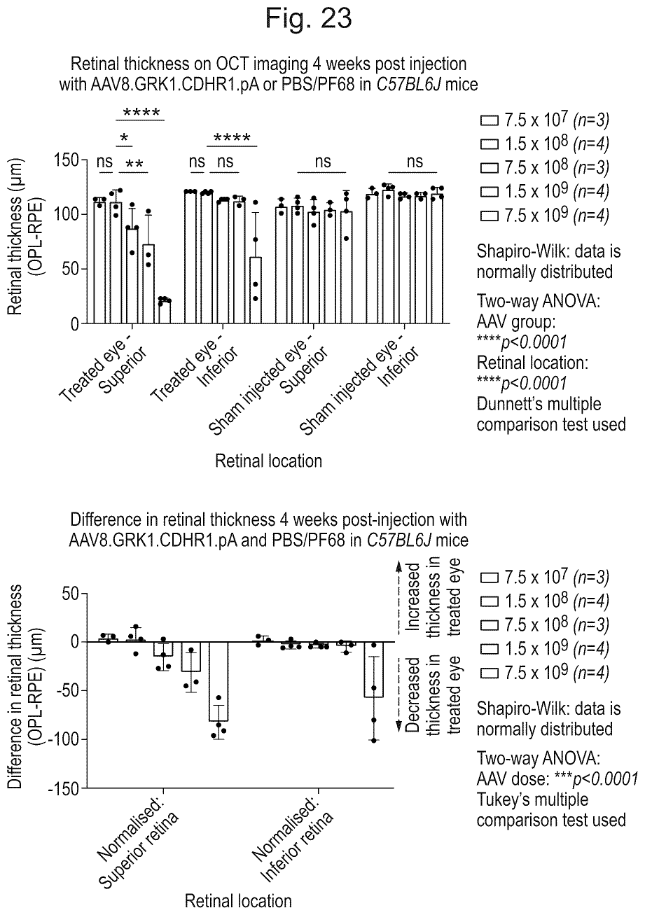

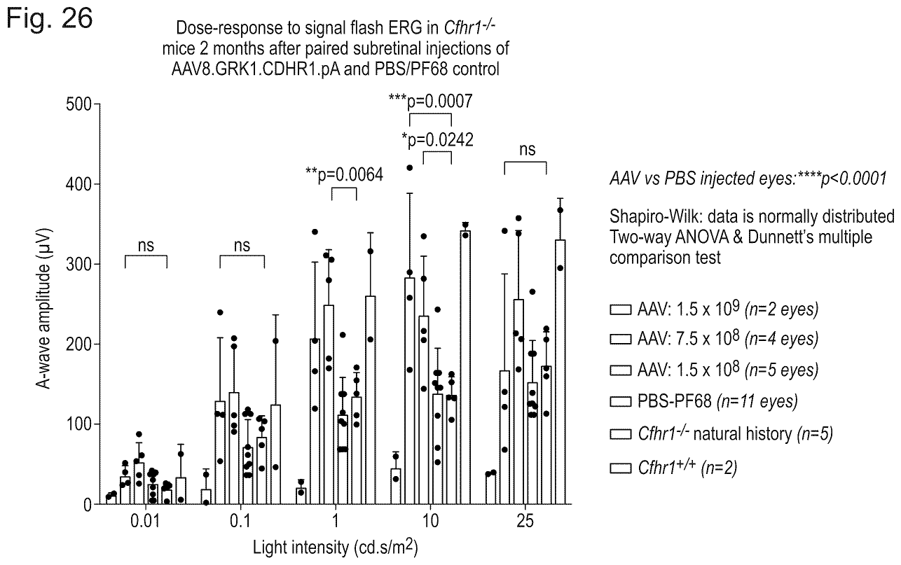

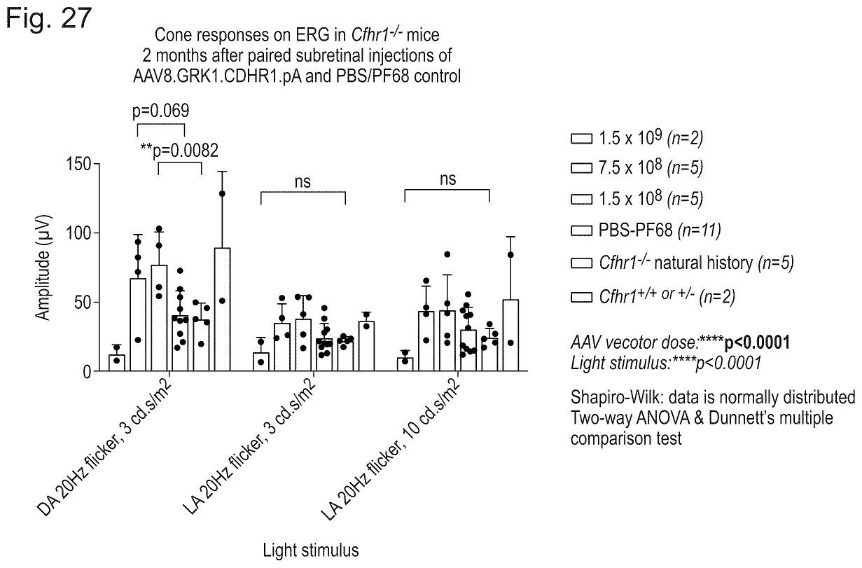

In Cdhr1^{-/-} mice, the maximum tolerated dose as evaluated by retinal thickness measurements 4-weeks following subretinal injection was 7.5×10⁸ gc (FIG. 22). Dose-dependent retinal thinning was seen at 1.5×10⁹ gc in some animals within the group, with an effect also seen at inferior retinal locations. In C57BL6J mice, a dose of 7.5×10⁸ gc was found to exhibit a mild degree of retinal thinning, affecting loci in the superior, but not the inferior retina (FIG. 23). Dark- and light-adapted electroretinography was undertaken 2 months following subretinal injection on 3 dosing groups (1.5×10⁸ gc to 1.5×10⁹ gc) to further guide dose selection in Cdhr1^{-/-} mice (FIGS. 26 and 27). Maximal dark-adapted and light-adapted responses were greater in AAV-treated eyes than paired PBS-injected control eyes and age-matched uninjected control eyes at 7.5×10⁸ gc (n=4) and 1.5×10⁸ gc (n=5) on both dark- and light-adapted responses (FIG. 26). A dose of 1.5×10⁸ gc was selected for an open label, prospective controlled trial since the rescue effect as seen on dark- and light-adapted electroretinography was greater than that seen in the 7.5×10⁸ gc dosing group. Furthermore, in C57BL6J mice, 1.5×10⁸ gc had not produced any demonstrable toxicity as evaluated by OCT imaging (FIG. 23; P=0.046, two-way ANOVA versus 7.5×10⁸ gc at superior retinal loci in AAV treated eyes).

### AAV8.In.GRK1.CDHR1.WPRE.pA

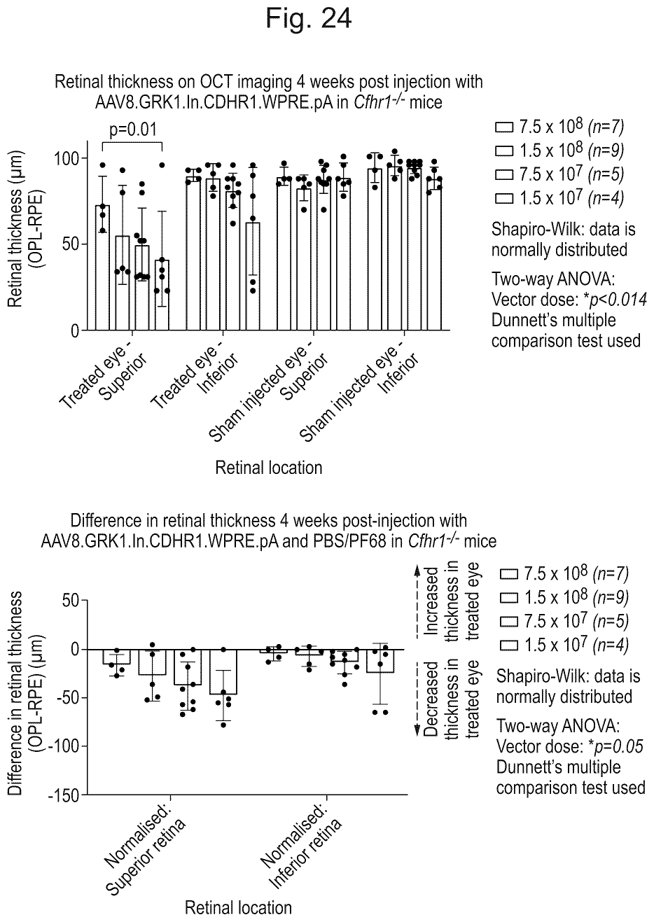

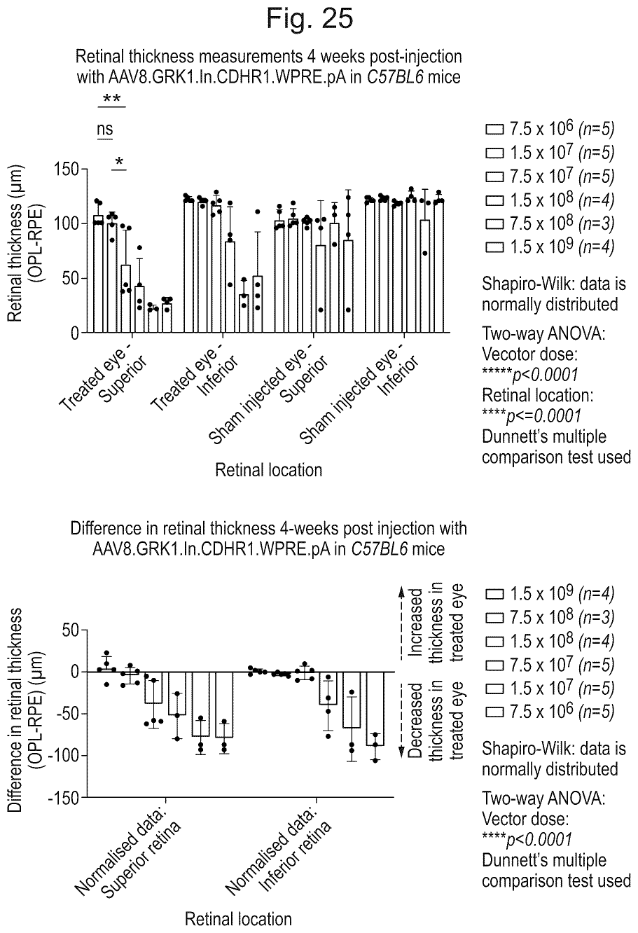

The inclusion of two cis-acting enhancing elements led to a higher degree of measured toxicity when compared to the unenhanced vector, as determined by retinal thickness measurements post-injection (FIG. 24-25).

In C57BL6J mice, subretinal delivery of 7.5×10⁶ gc to 1.5×10⁷ gc did not demonstrate retinal thinning versus PBS-injected control eyes (P>0.95 for both doses, two-way ANOVA). In Cdhr1^{-/-} mice, a dose of 1.5×10⁷ gc did not produce more retinal thinning than seen in PBS-injected control eyes (P=0.32, two-way ANOVA), although this was seen at doses of 7.5×10⁷ gc or higher. Although a safe dosing limit was thus identified, time constraints led to the selection of AAV8.GRK1.CDHR1.pA for a longitudinal gene rescue experiment.

### Example 4—CDHR1 Gene Therapy Study: Experimental Design

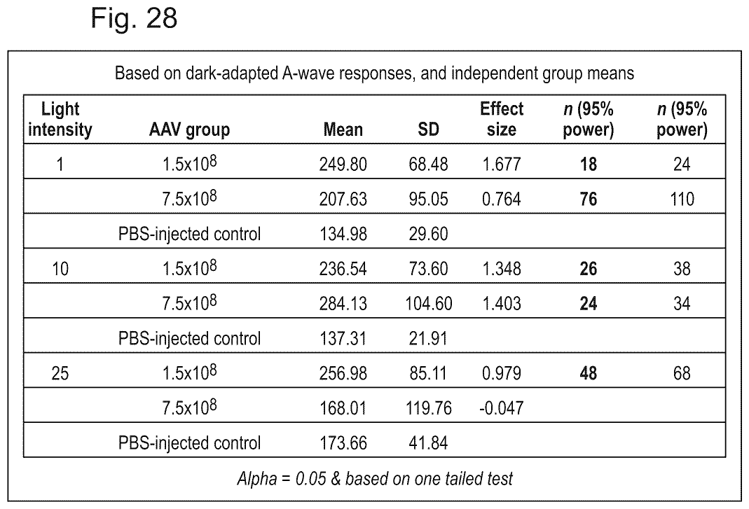

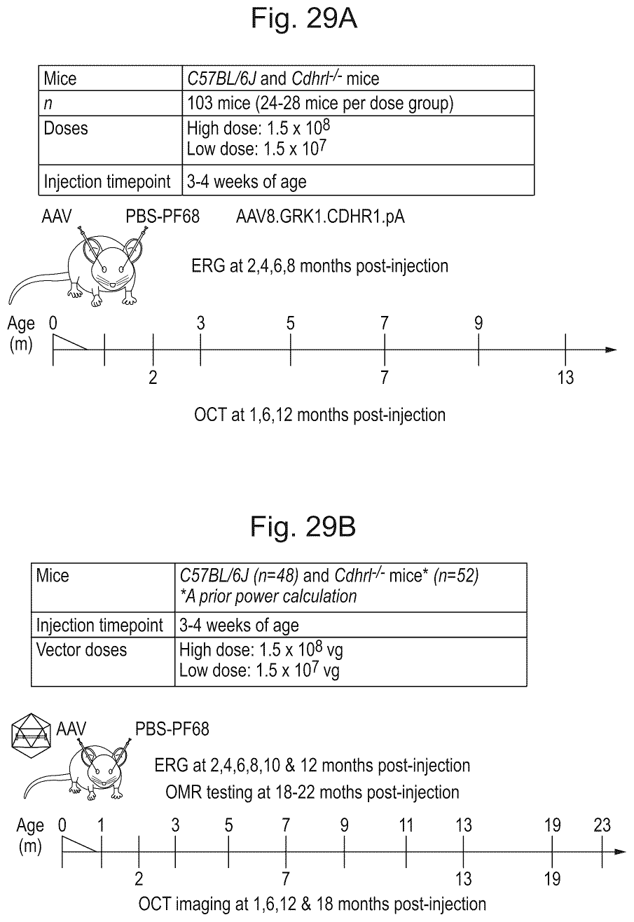

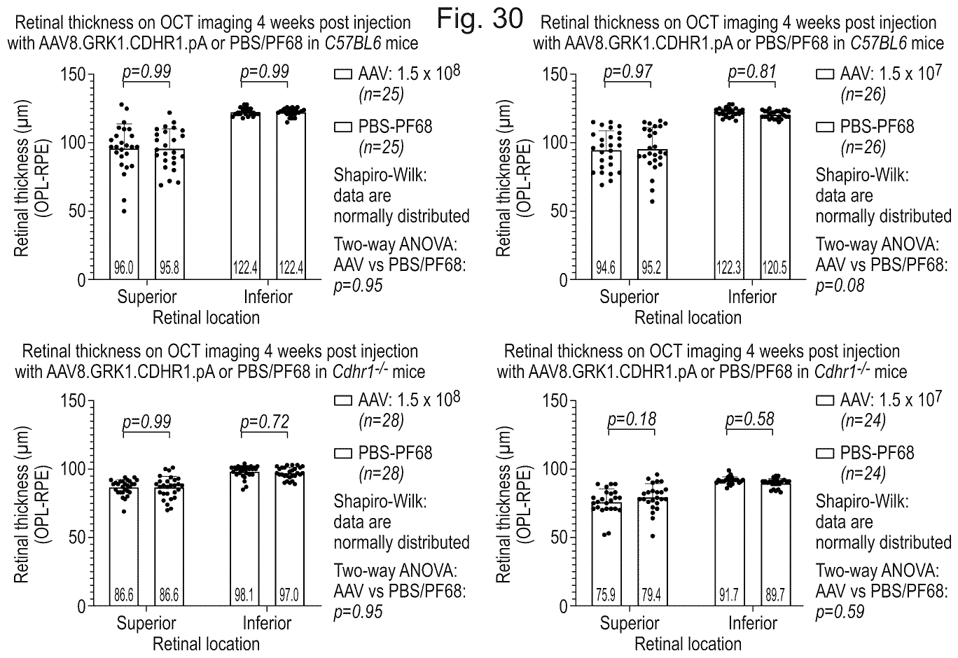

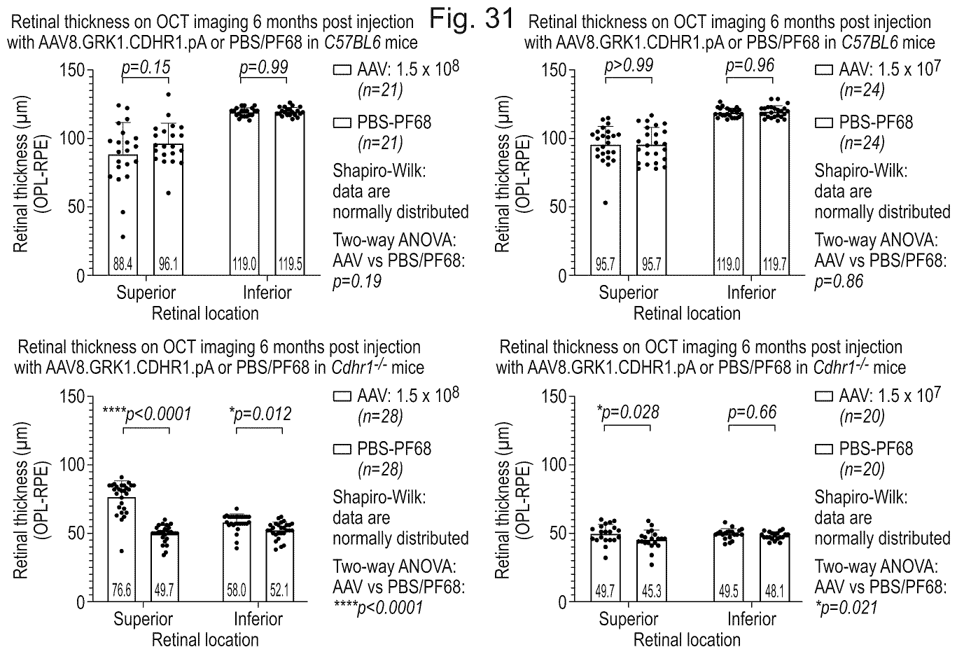

A prospective, open-label, paired controlled trial was undertaken to evaluate the safety and efficacy of the AAV8.GRK1.CDHR1.pA vector on retinal structure and function in Cdhr1^{-/-} and C57BL6J mice (experimental design is shown in FIG. 29). A priori power calculations determined that 26 mice would detect a difference between AAV- and PBS-injected paired control eyes with 95% power, based on maximal dark- and light-adapted A-wave responses seen on electroretinography in the Cdhr1^{-/-} dosing study at 1.5×10⁸ gc (FIG. 28). Maximal dark-adapted ERG responses had not differed between the doses of 1.5×10⁸ gc and 7.5×10⁸ gc in a pilot experiment (P>0.97 for all light intensities on dark-adapted luminance series; data not shown). Two doses of AAV8.GRK1.CDHR1.pA vector were evaluated: the high-dose group (1.5×10⁸ gc) and low-dose group (1.5×10⁷ gc) since it was determined that a log unit interval in dose would likely identify a difference in dose-response between the two groups.

Animals in both Cdhr1^{-/-} and C57BL6J groups underwent sub-retinal injection of AAV8.GRK1.CDHR 1.pA with PBS vehicle control in the fellow eye at 3-4 weeks of age (post-weaning). The subretinal injection volume was sufficient to detach the superior hemi-retina, leaving the inferior retina attached as an internal control for retinal imaging studies which can evaluate both retinal locations. OCT imaging was undertaken at 4-weeks post-injection to determine the effect of surgically induced retinal detachment on outer retinal thickness measurements, and to provide a baseline measurement for comparison with later time-points. Additional OCT imaging was undertaken at 6-months post-injection in all groups. Dark- and light-adapted electroretinography was undertaken at 2-, 4- and 6-months post-injection.

In the high-dose group (1.5×10⁸ gc), an extension study was undertaken with electroretinography performed at 8-, 10- and 12-months post-injection with additional OCT imaging performed at 12- and 18-months post-injection and OMR testing at 18 to 22 months post-injection for both Cdhr1^{-/-} and C57BL6J groups (FIG. 29(b)).

### Example 5—Sub-Retinal Delivery of AAV8.CDHR1 is Safe in C57BL6J Mice

At 4-weeks post-injection, outer retinal thickness measurements did not differ between paired AAV- and PBS-injected eyes at equivalent superior retinal loci (P=0.99 for 1.5×10⁸ gc and P=0.97 for 1.5×10⁷ gc, two-way ANOVA) on OCT imaging in C57BL6J mice (FIG. 30). Retinal thinning and increased variability in retinal thickness measurements were seen at superior versus inferior locations in both AAV- and PBS-injected eyes at both doses due to the effect of surgically induced retinal detachment. At 6-months post-injection (FIG. 31), there was no difference between retinal thickness measurements in AAV-injected and PBS-injected controls (P>0.99 for 1.5×10⁷ gc, and P=0.15 for 1.5×10⁸ gc; two-way ANOVA). At 12-months post-injection, further analysis in the high dose group showed no difference in mean superior retinal thickness measurements (85.5 μm in AAV-treated eyes versus 88.9 μm in PBS-injected eyes; P=0.77, two-way ANOVA).

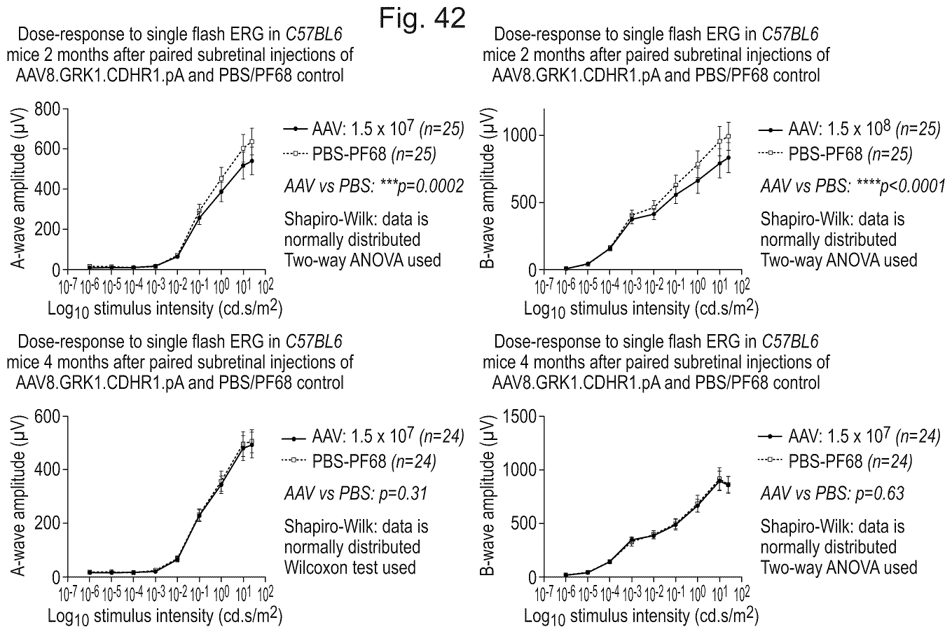

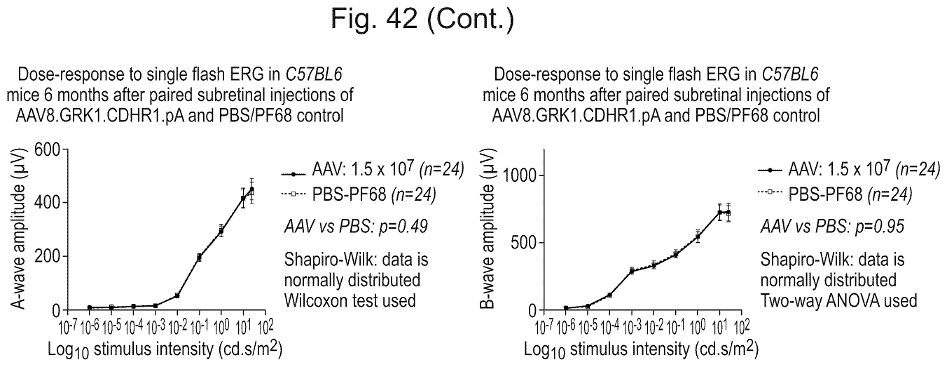

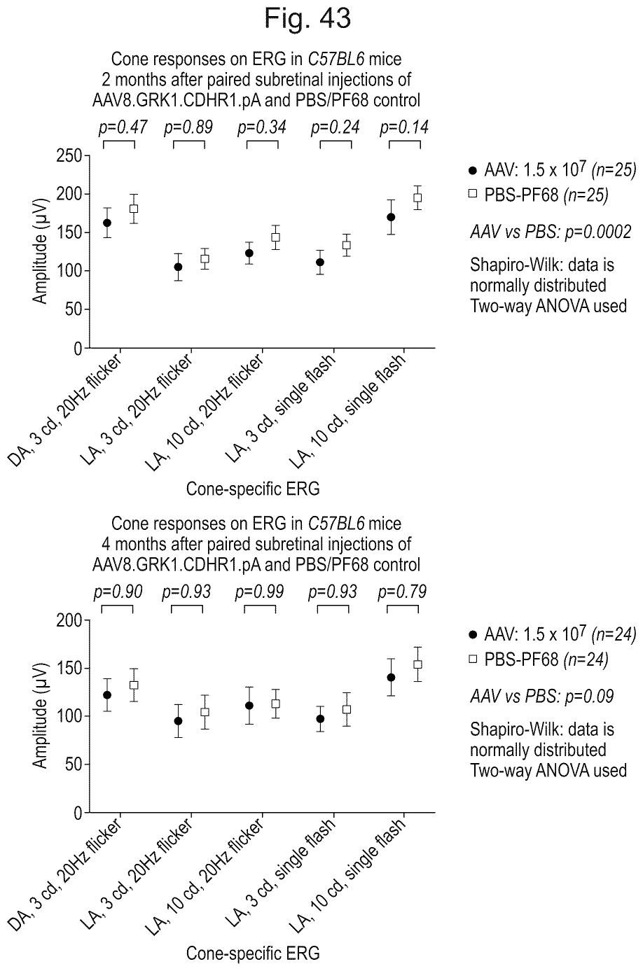

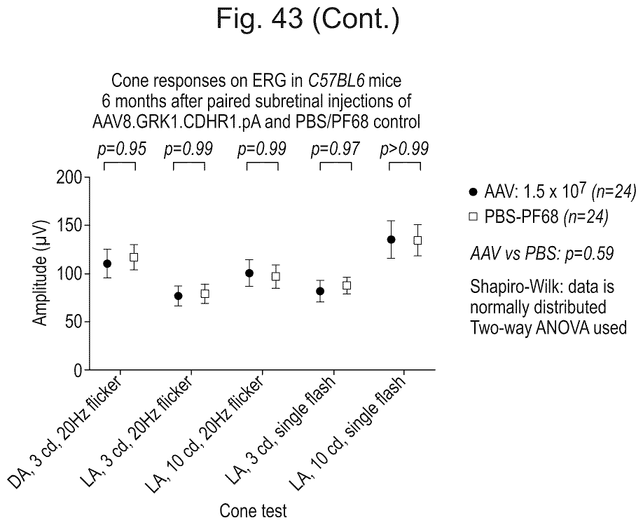

Dark-adapted electroretinography did not detect a difference between low dose (1.5×10⁷ gc) AAV- and PBS-injected paired control eyes at 6 months post-injection (FIG. 42) when comparing A-wave amplitudes (P=0.49) and B-wave amplitudes (P-0.95; two-way ANOVA for both analyses). Similarly, light-adapted ERG recordings did not detect a difference between groups on 20 Hz cone flicker at 10 cd·s/m² (P=0.99), or single-flash responses at the same intensity (P>0.99). In the high-dose group at 6-months, there was no significant difference in A-wave amplitudes (P=0.65; two-way ANOVA) and B-wave amplitude (P=0.47) between AAV- and PBS-injected eyes. Similarly, light-adapted responses were not significantly different (P=0.98 for light-adapted flicker and P=0.69 for light-adapted single-flash responses at 10 cd·s/m²). Overall, these data show that at the chosen doses, AAV8.GRK1.CDHR1.pA is safe when delivered to the wildtype retina by detailed structural and functional measures.

### Example 6—AAV8.CDHR1 Rescues Rod and Cone Photoreceptor Function in Cdhr1^{-/-} Mice

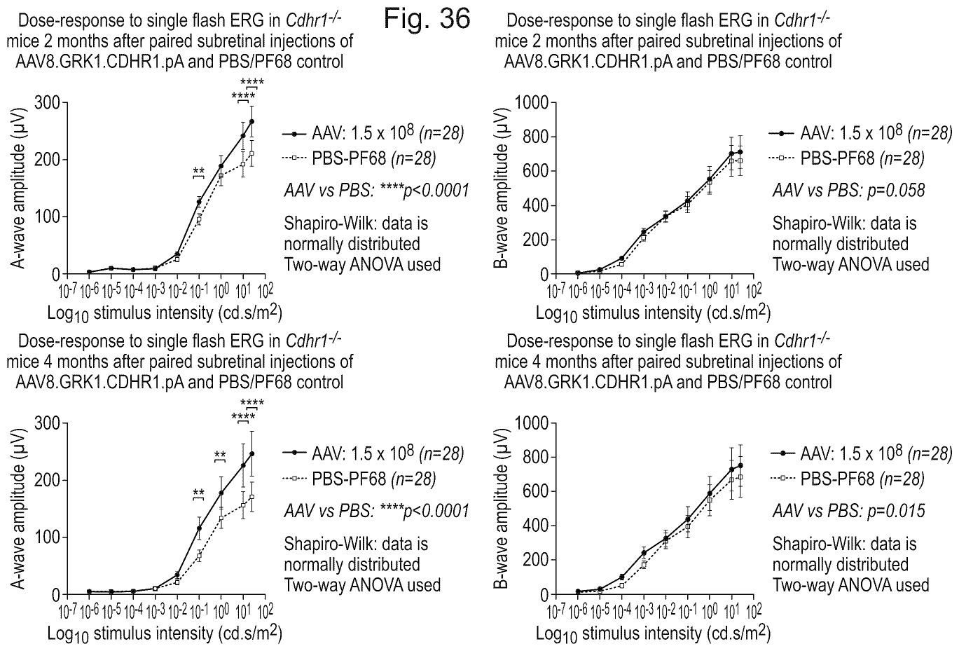

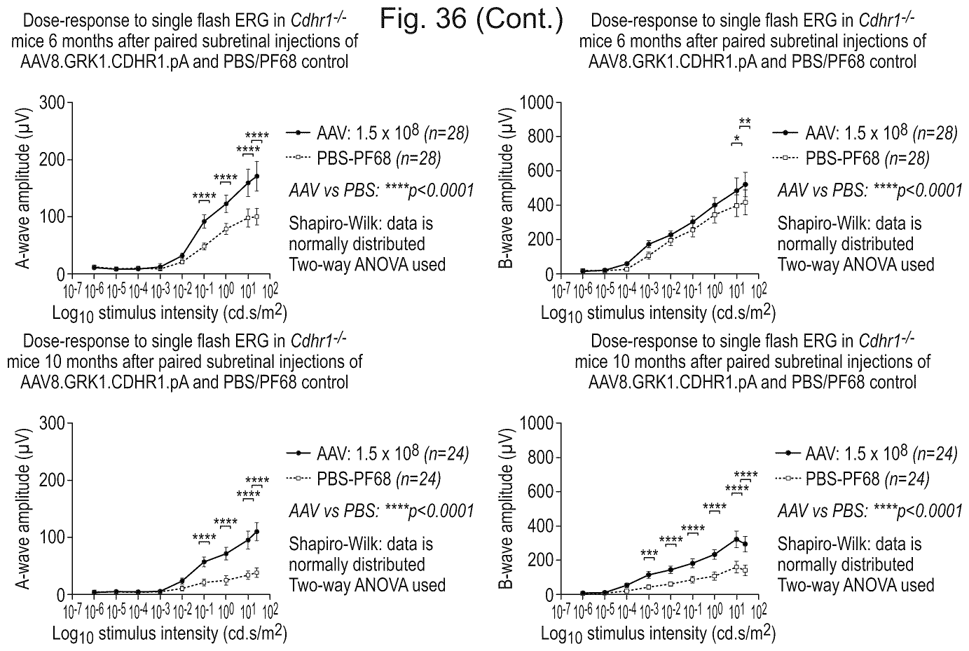

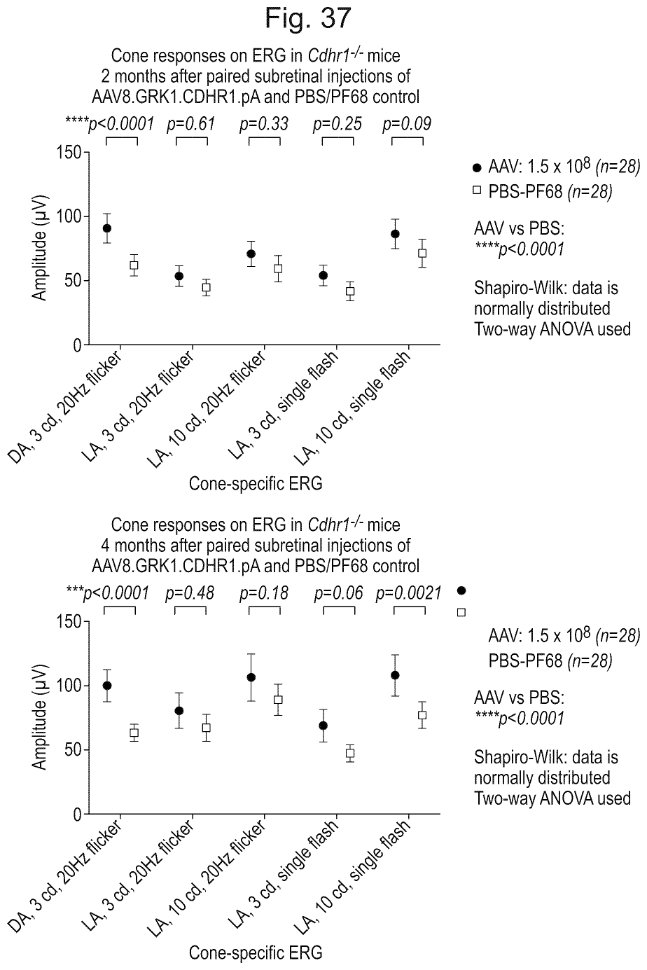

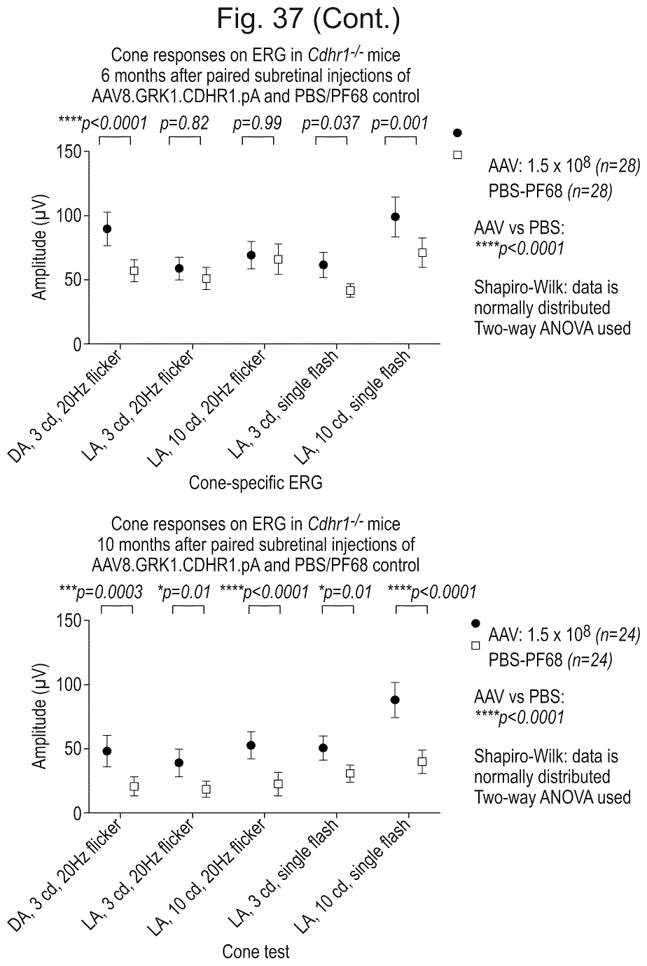

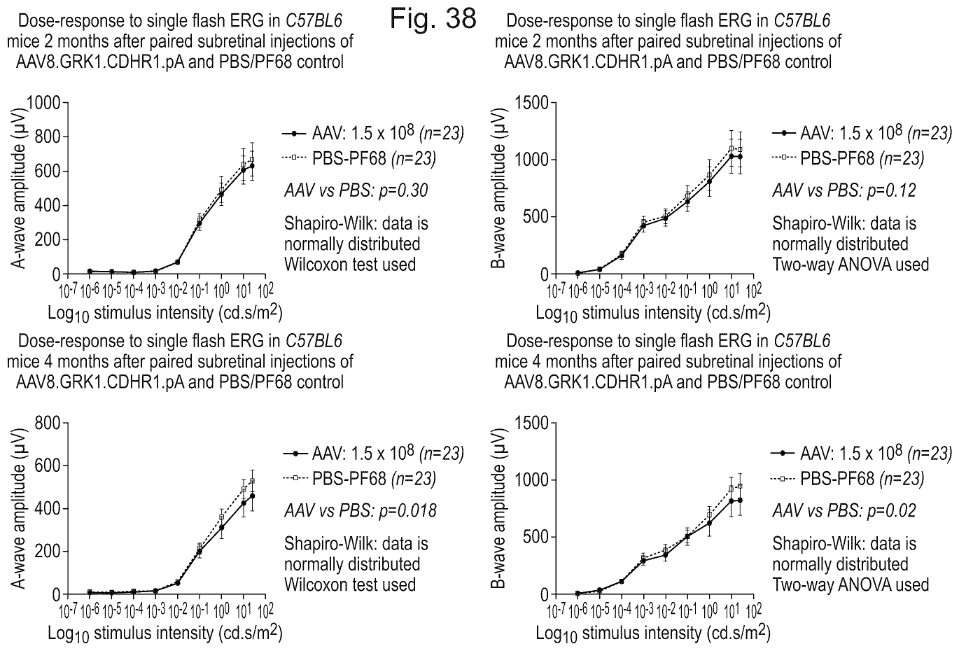

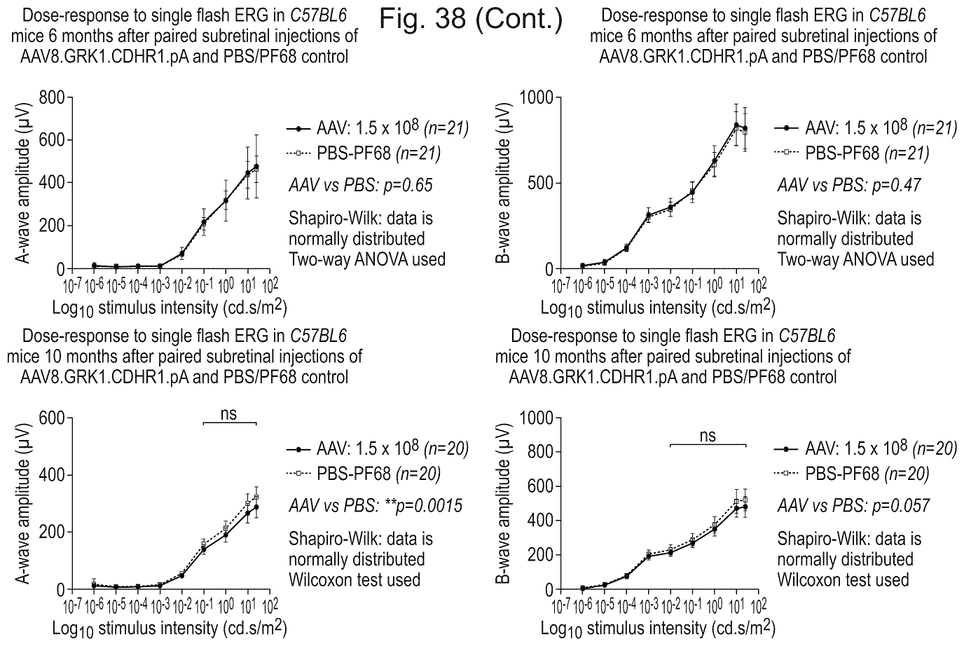

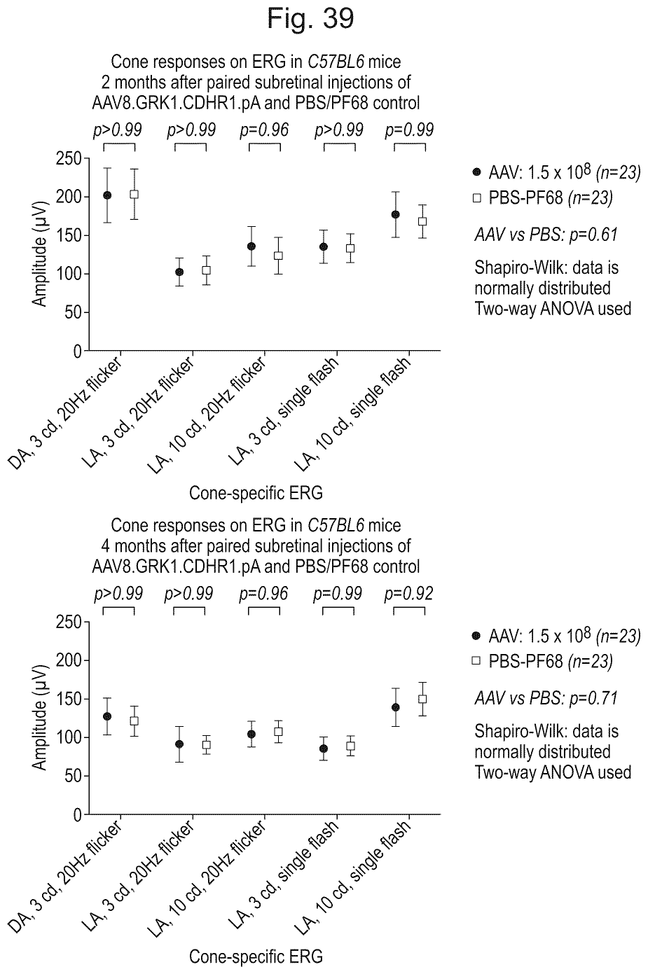

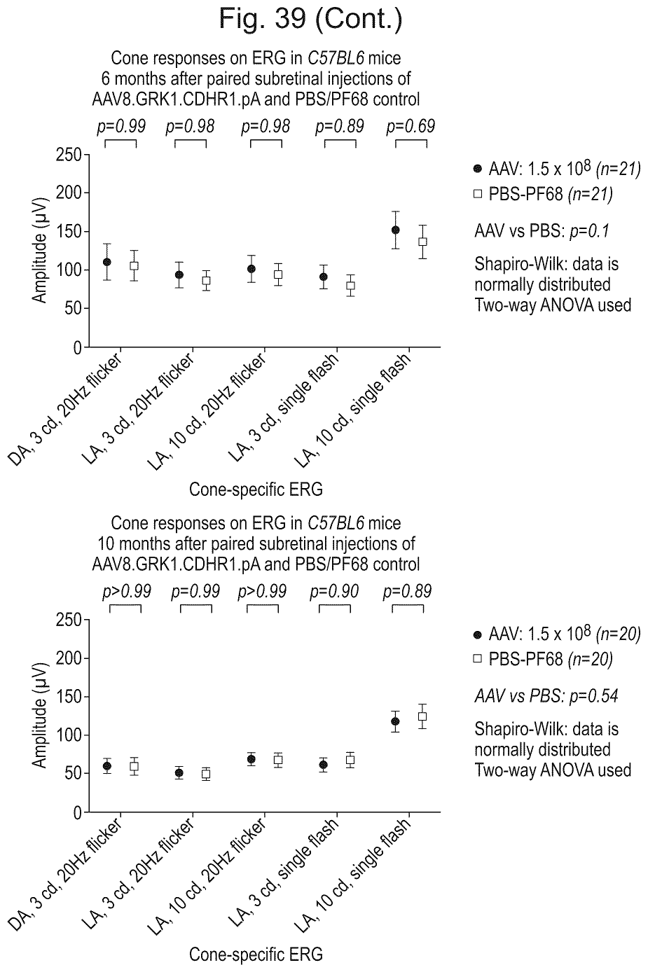

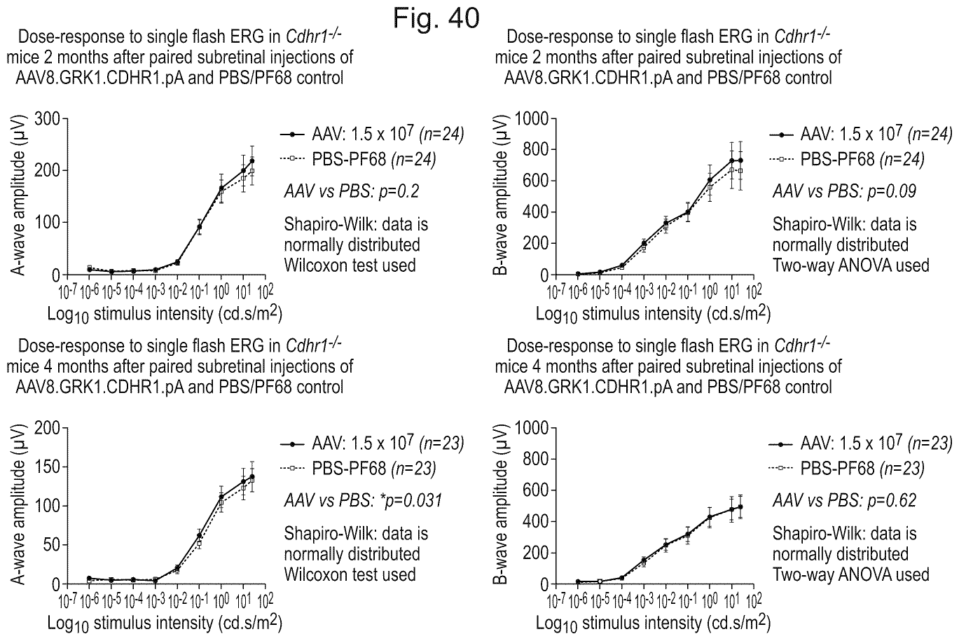

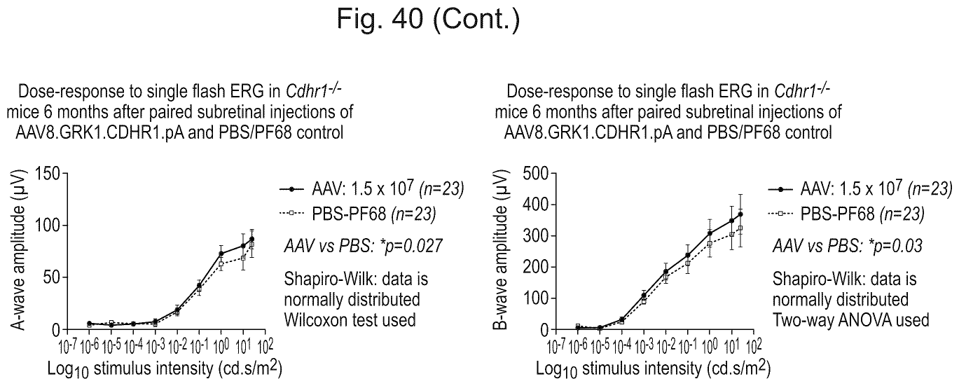

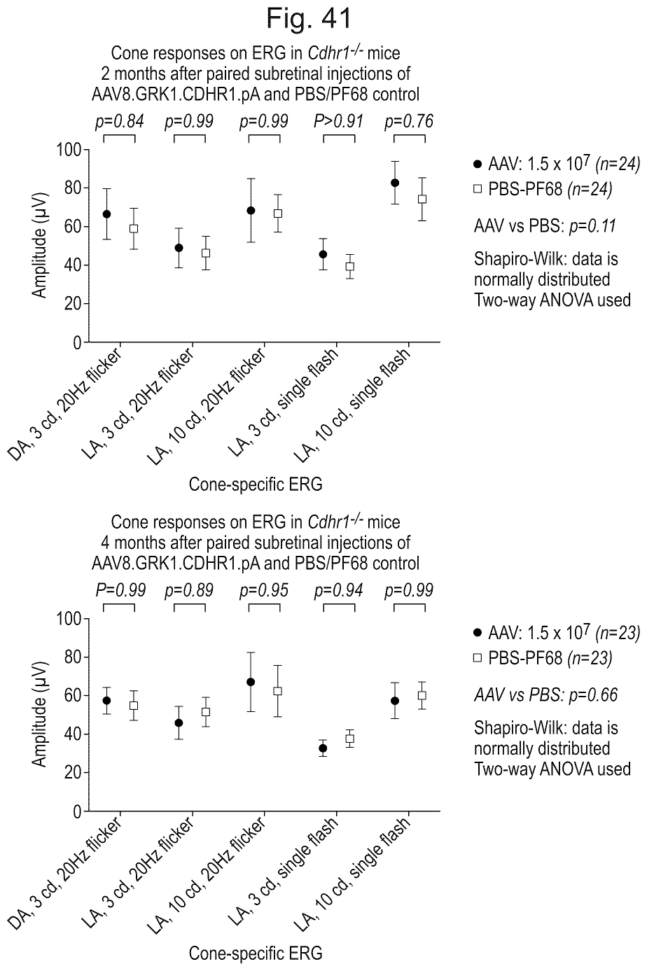

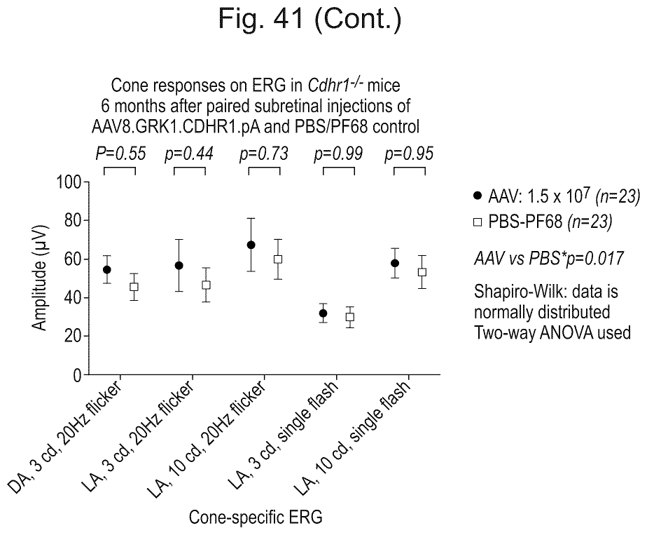

The dark-adapted electroretinography luminance series identified a benefit of sub-retinal AAV8.GRK1.CDHR1.pA as early as 2-months post-injection when A-wave responses were significantly improved compared to PBS-injected control eyes (P<0.0001 across all light intensities; two-way ANOVA; FIG. 36). Over 10 months, the difference between A-wave responses between AAV-treated and control eyes increased incrementally at each successive timepoint. B-wave responses were not significantly different between groups at 2 months (P=0.058), but were significantly improved at 4 months (P=0.015), and thereafter (P<0.0001 after 4 months). Light-adapted responses were greater overall at all timepoints in AAV versus PBS treated eyes (P<0.0001 overall; two way ANOVA; FIG. 37). The light-adapted single-flash responses at 10 cd·s/m² were significantly improved in AAV-injected eyes at 4 months (P=0.0021) and later timepoints (P=0.001 or less). Light-adapted 20 Hz flicker responses at the same intensity were significantly improved in AAV-injected eyes at 8 months and at later timepoints (P<0.0001). At 10-months post-injection, cone responses were significantly improved across all tests in the AAV-injected eyes (P<0.05 for all tests; FIG. 37).Dark-adapted ERG responses in the low-dose AAV8.GRK1.CDHR1.pA (1.5×10⁷ gc) group were not significantly different at 2-months (FIG. 40). However, A-wave responses were significantly improved at 4- and 6-months (P=0.031 and P=0.027 for both time-points), although the effect size was small. B-wave responses were significantly improved at 6 months in AAV-injected eyes (P=0.03). Light-adapted responses were not different between AAV-injected and PBS-injected eyes at the low dose at any timepoint, although were significantly improved overall at 6 months (P=0.017).

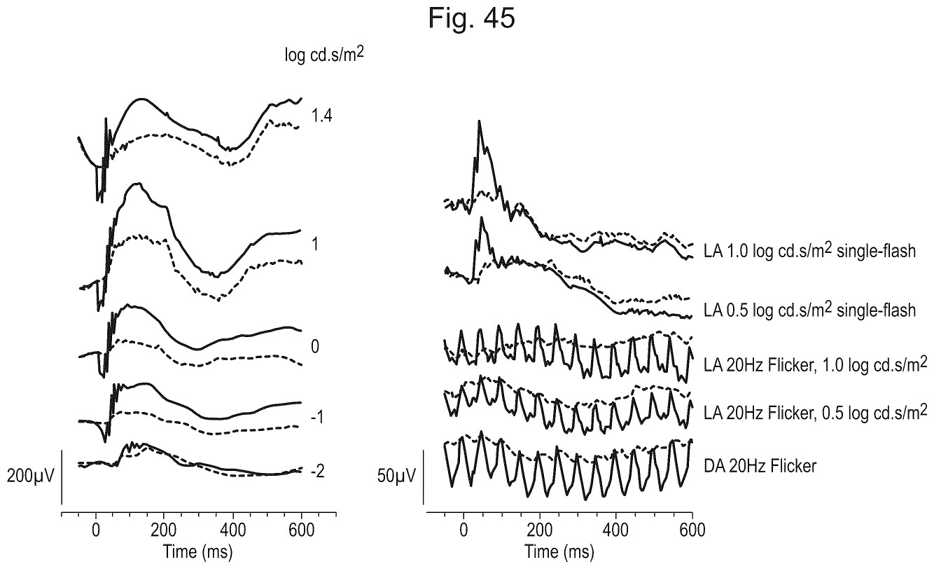

Raw ERG traces recorded simultaneously from paired Cdhr1−/− eyes 12-months post-injection illustrate the benefit of CDHR1 gene therapy (FIG. 45). A-wave responses, representing rod and combined rod-cone photoreceptor responses, are present in AAV-injected eyes only at this timepoint. Moreover, B-wave responses (ON-bipolar cells) are significantly larger and oscillatory potentials (amacrine cells) are only discernible in AAV-treated eyes.

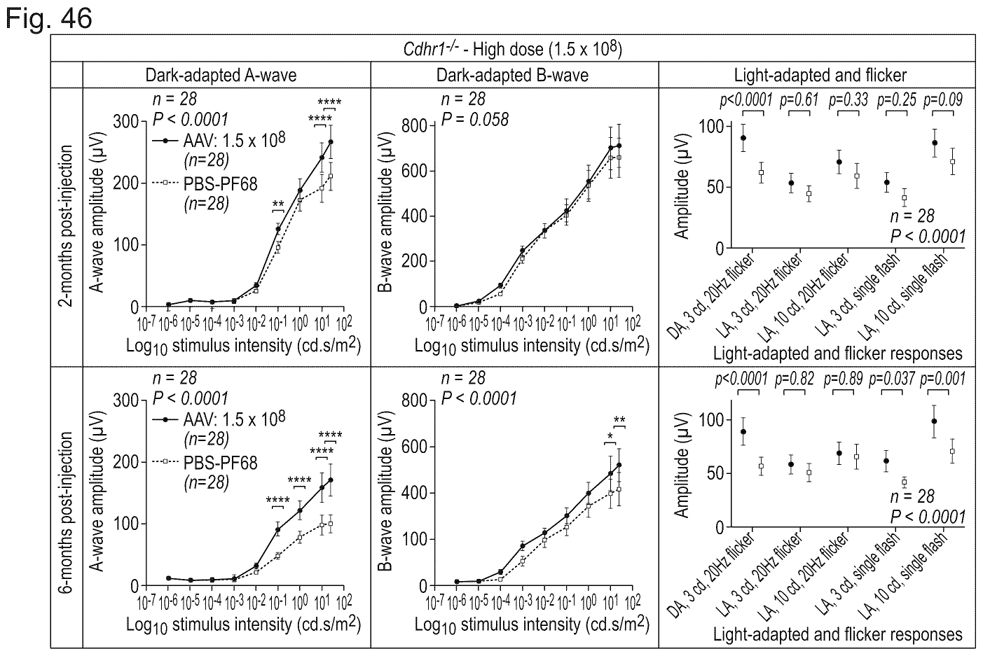

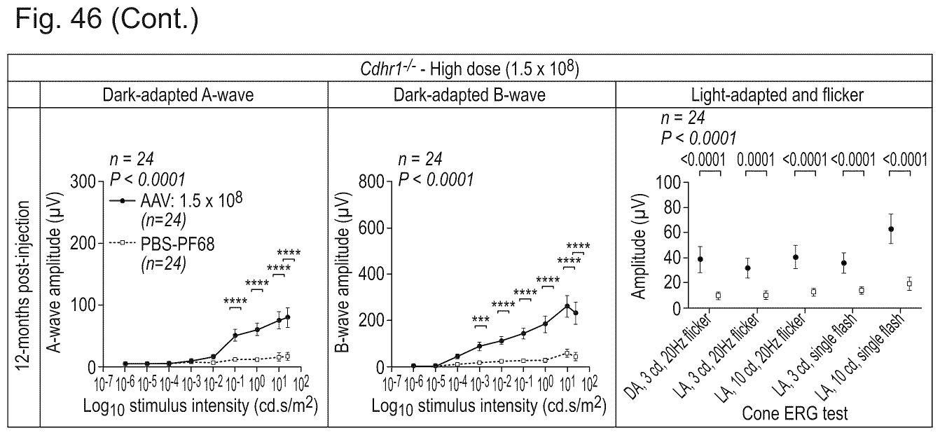

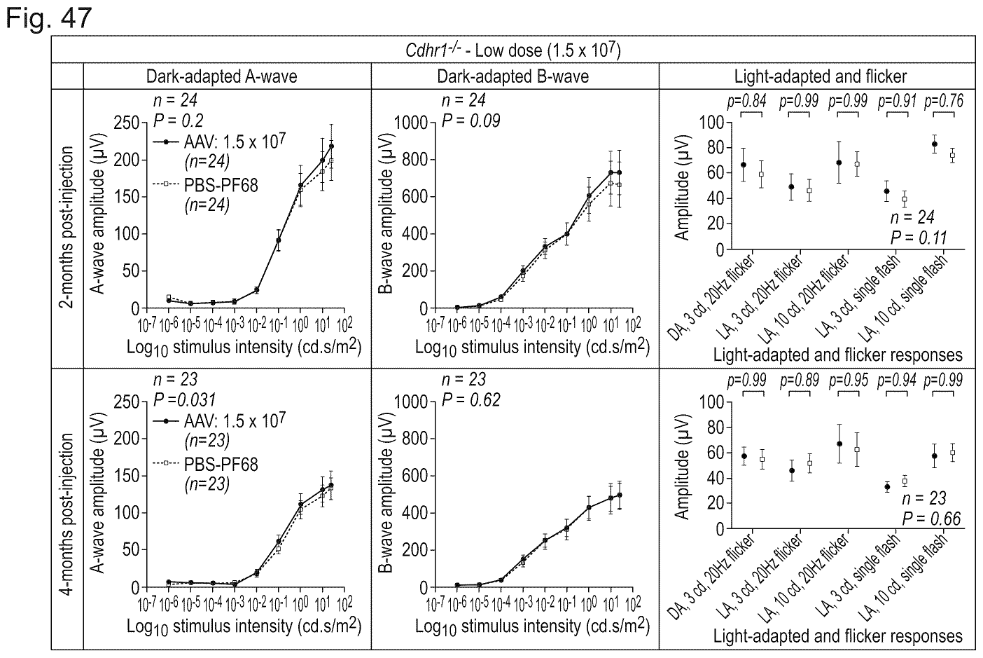

Following high-dose AAV8.GRK1.CDHR1.pA, a significant benefit was seen in dark-adapted flicker responses at 2-month post-injection compared to PBS-injected control eyes (P<0.0001; n=28). At this early timepoint, other light-adapted single flash and flicker response amplitudes were better preserved in AAV- versus PBS-injected eyes, although not significant individually (FIG. 46). At 4-months post-injection, light-adapted single-flash response amplitudes were better preserved in AAV-injected eyes (P=0.0021; n=28—data not shown), with increasing effect size at each successive timepoint. Light-adapted flicker response amplitudes were significantly larger in AAV-injected eyes from 8 months post-injection at both 3 cd·s/m² (P=0.037; n=26) and 10 cd·s/m² (P=0.0003), which became more significant at later timepoints (P<0.0001 at 12-months for both light-adapted flicker responses; n=24) (FIG. 46).

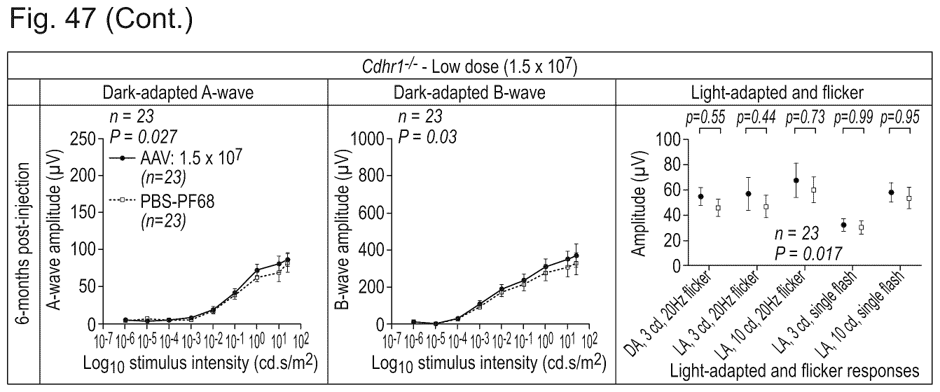

In the low dose group, although mean response amplitudes were greater in AAV-injected n=23), multiple comparison testing failed to detect a significant benefit in any individual light-adapted or flicker ERG test (FIG. 47).

Raw light-adapted ERG traces recorded simultaneously 12-months post-injection in a single Cdhr1−/− mouse illustrate the benefits of high-dose AAV-injection on cone photoreceptor function that is sustained to at least 12-months post-injection (FIG. 45). Light-adapted single-flash and flicker responses are observed in the AAV-injected eye and near-absent in the fellow PBS-injected eye.

### Example 7—Shortened A-Wave Implicit Times in Cdhr1−/− Mice Following High-Dose AAV8.GRK1.CDHR1.pA at 12-Months Post-Injection

### Dark-Adapted A-Wave Implicit Times

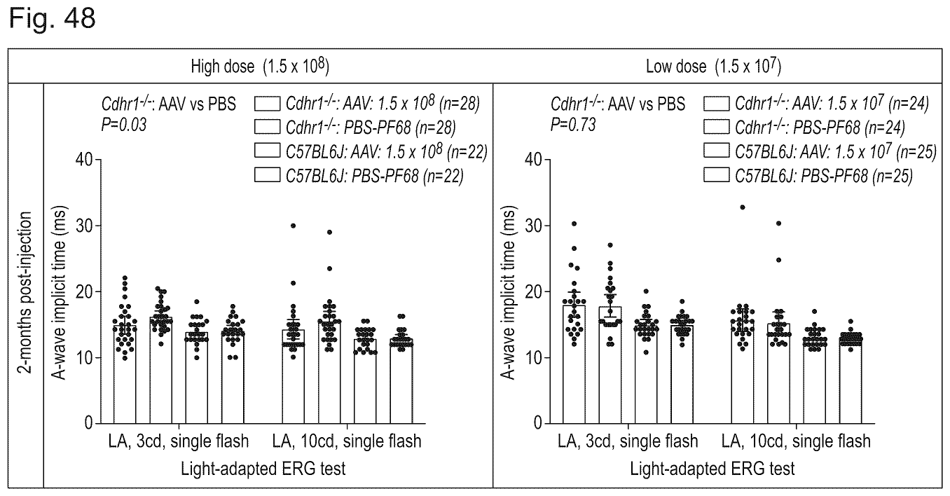

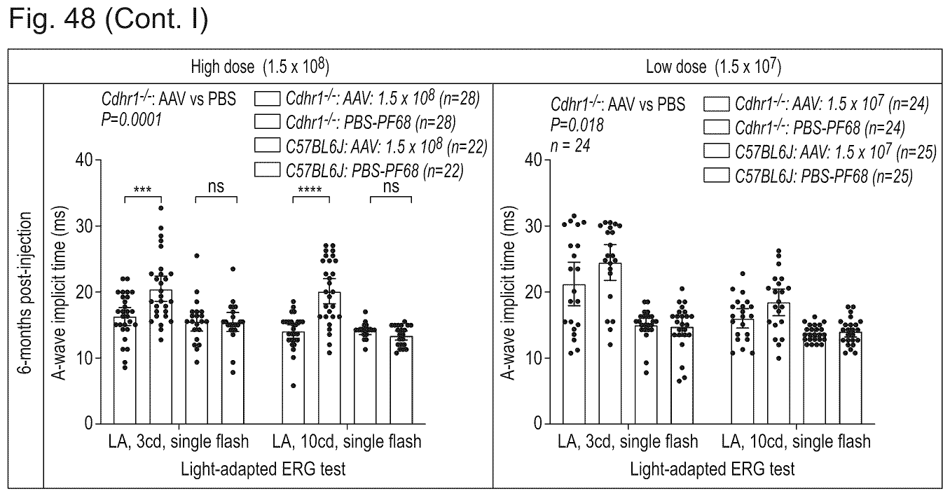

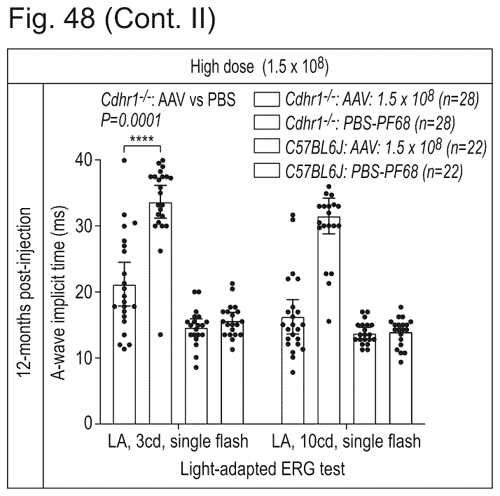

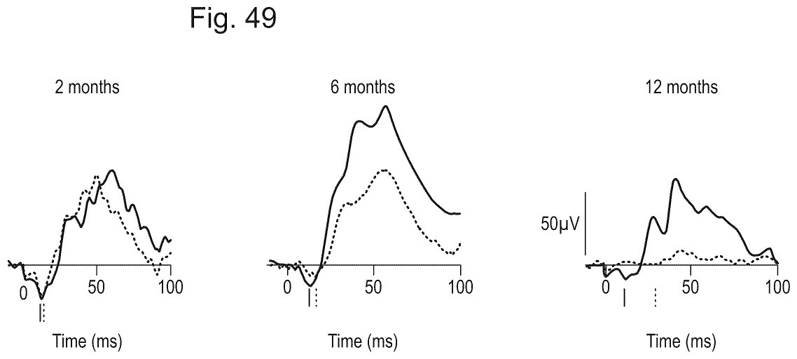

Dark-adapted A-wave implicit times were significantly shorter in AAV- versus PBS-injected Cdhr1−/− eyes (mean, 6.0 vs. 10.2 ms at 25 cd·s/m²) at 2-months post- injection in the high-dose group (P<0.0001 at all light intensities; n=28), a difference which increased to 12-months post-injection when A-waves were not easily discernible in PBS-injected eyes (FIGS. 48 and 49). Moreover, A-wave implicit times in AAV-injected Cdhr1−/− eyes were not significantly different from AAV-injected C57BL/6J eyes at the highest light intensities at 2-months post-injection (P>0.92), although this difference became significant at later timepoints.

Conversely, A-wave implicit times were significantly longer in Cdhr1−/− versus C57BL/6J eyes (P<0.0001 overall, and at the 4 highest light intensities) at 2-months following PBS injection. In the low dose group, A-wave implicit times were not significantly different between AAV- and PBS-injected eyes to 6-months post-injection in Cdhr1−/− mice (P=0.43; n=22) (FIG. 48).

### Light-Adapted Single-Flash A-Wave Implicit Times

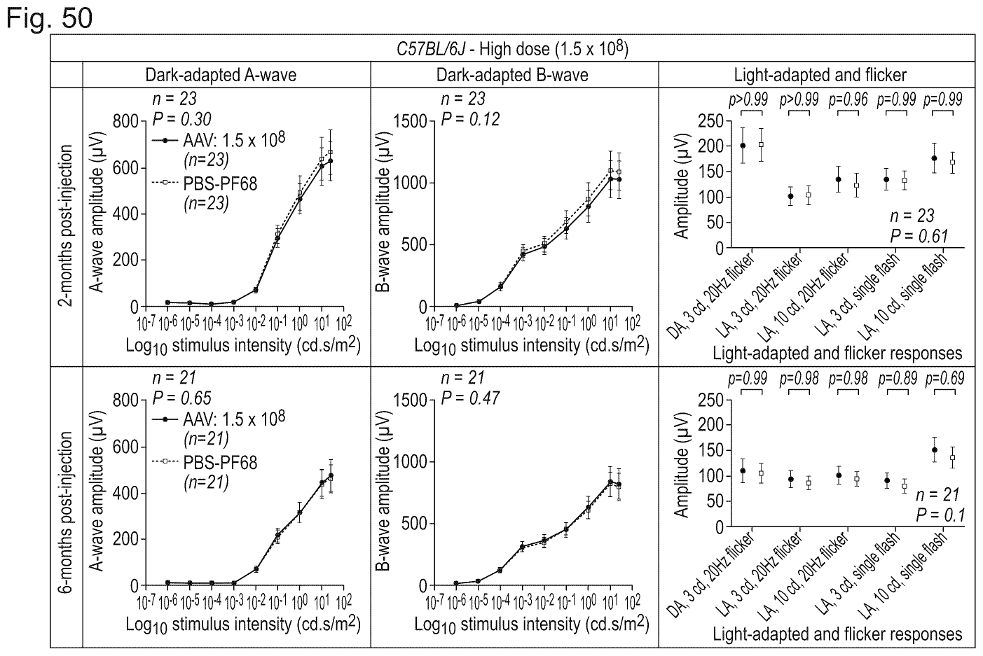

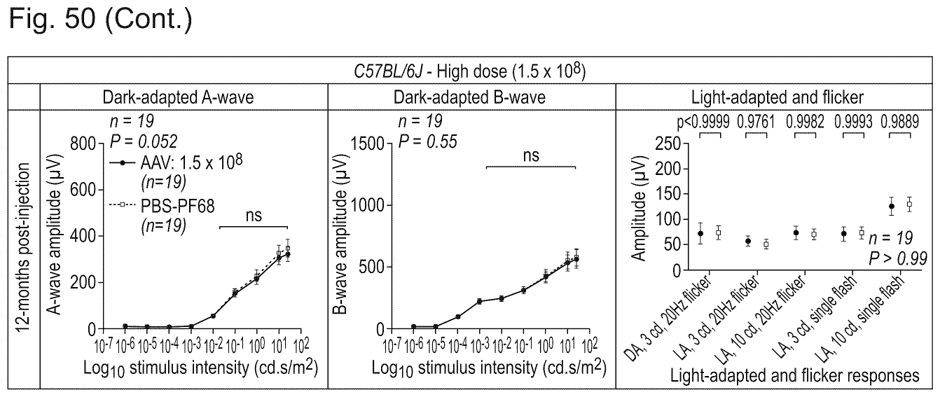

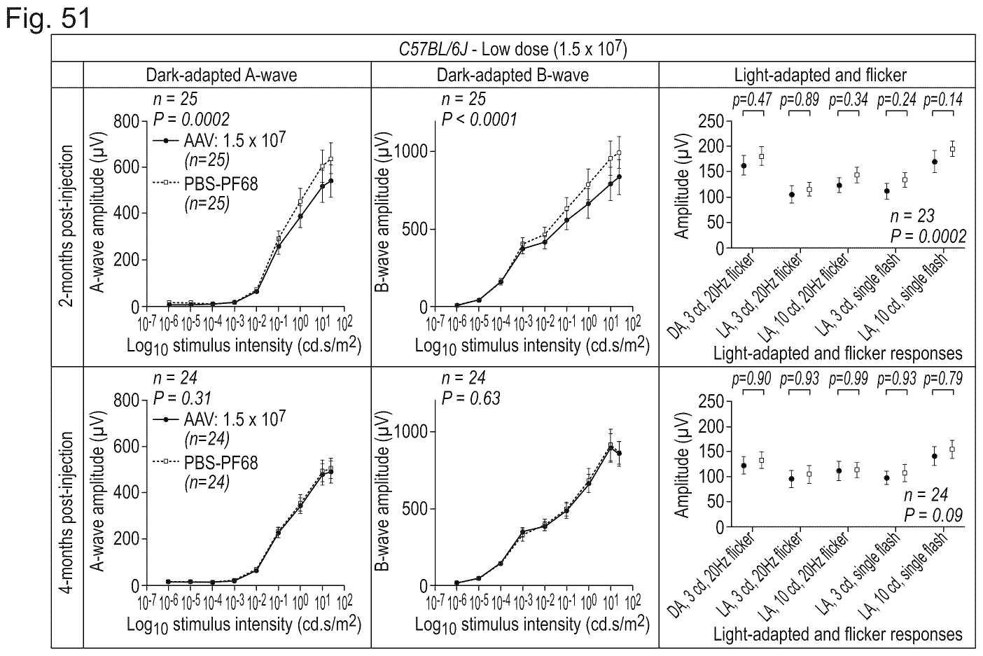

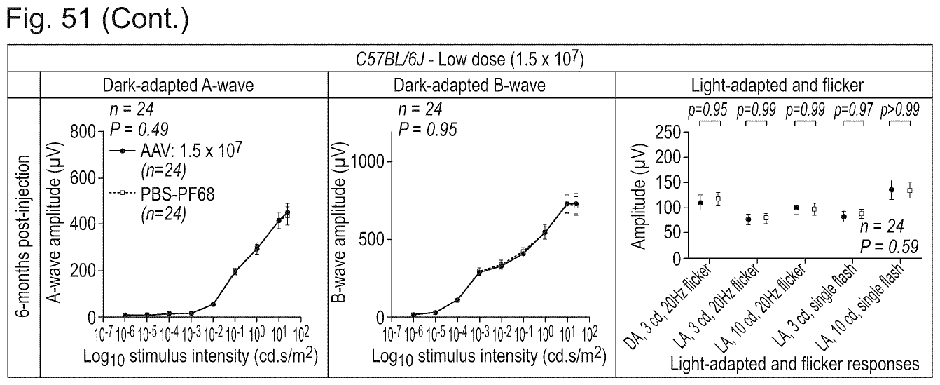

Sub-retinal injection of AAV8.GRK1.CDHR1.pA at a dose of 1.5×10⁸ shortened A-wave implicit times on light-adapted, single flash ERG versus PBS-injected control eyes at 2-months post-injection (P=0.03; n=28) in Cdhr1−/− mice (FIGS. 50 and 51). The difference in light-adapted A-wave implicit times between treated and control eyes increased further at 6-months (P<0.0001; n=28) and 12-months (P<0.0001; n=24). The rescue effect was highly significant at two light intensities at the latest timepoint, although was more prominent at 10 cd·s/m² where implicit times at 12-months were not significantly different from AAV-injected C57BL/6J eyes (P=0.23; two-way ANOVA for the effect of treatment with Šídák's multiple comparison test for all statistics presented in this section).

In the low dose group, A-wave implicit times on light-adapted single flash ERG were no different in AAV and PBS-injected eyes at 2 months (P=0.73, n=24). However, A-wave implicit times were shorter in AAV-injected eyes at 6 months post-injection overall (P=0.018; n=22) (FIG. 50), although were not significant for individual light intensities.

### Example 8—Electroretinography Excludes Cone and Rod Dysfunction in C57BL/6J Mice to 12 Months Post-Injection of AAV8.GRK1.CDHR1.pA

### Dark-Adapted ERG

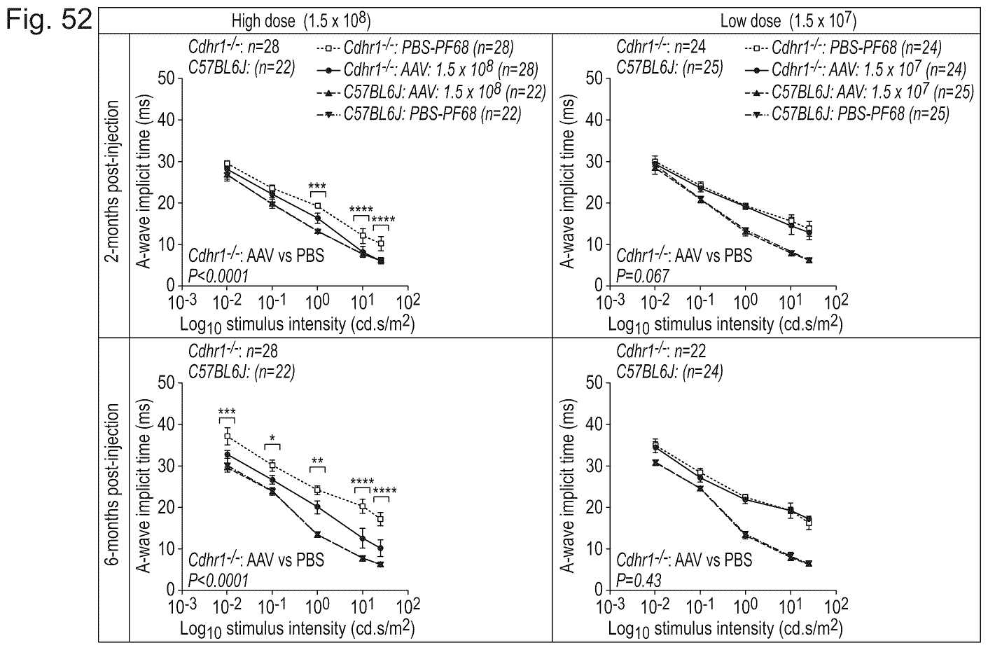

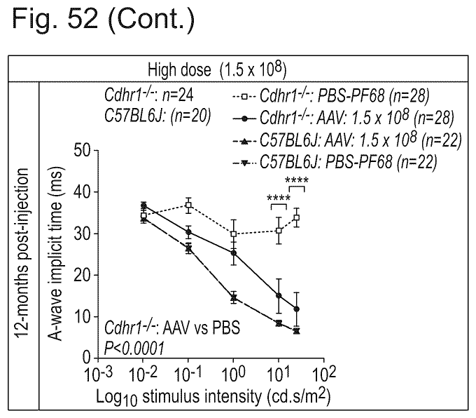

In the high-dose group, there was no difference in A-wave amplitudes (P=0.052; n=19) and B-wave amplitudes (P=0.55) in C57BL/6J mice 12-months post- injection, with no statistical difference between amplitudes at any light intensity on multiple comparison testing. The two groups were well-matched at all earlier timepoints (FIG. 50). Similarly, in the low dose group, there was no difference between AAV- and PBS-injected eyes in A- (P=0.49; n=24) or B-wave (P=0.95) amplitudes at 6-month post-injection (FIG. 51), the latest timepoint tested. A-wave implicit times, were no different between AAV- and PBS-injected eyes to 6-months (low dose) and 12-months post-injection (high dose) in C57BL/6J mice on dark-adapted ERG (P>0.99 at all light intensities) (FIG. 52).

### Light-Adapted ERG

There was no difference in light-adapted single-flash and flicker response amplitudes between high dose AAV- and PBS-injected eyes to 12-months post injection in C57BL/6J mice (P>0.99 overall; n=19), with no differences in individual ERG tests of the cone system on multiple comparison testing (P>0.97 for all tests) (FIG. 50). In the low dose cohort, there was no difference in responses between AAV- and PBS-injected eyes to 6-months post-injection (P=0.59 overall; n=24) (FIG. 51), with no differences on individual tests (P>0.95 for all).

A-wave implicit times on light-adapted, single-flash ERG at both 3 and 10 cd·s/m², were no different between AAV- and PBS-injected eyes in C57BL/6J mice in both low- (P=0.96) and high-dose (P=0.26) groups at 6- and 12-months post-injection, respectively (FIG. 48).

### Example 9—High-Dose AAV8.GRK1.CDHR1.pA Rescues Photopic and Scotopic Optomotor Reflex Responses in Cdhr1−/− Mice to 21-Months Post-Injection

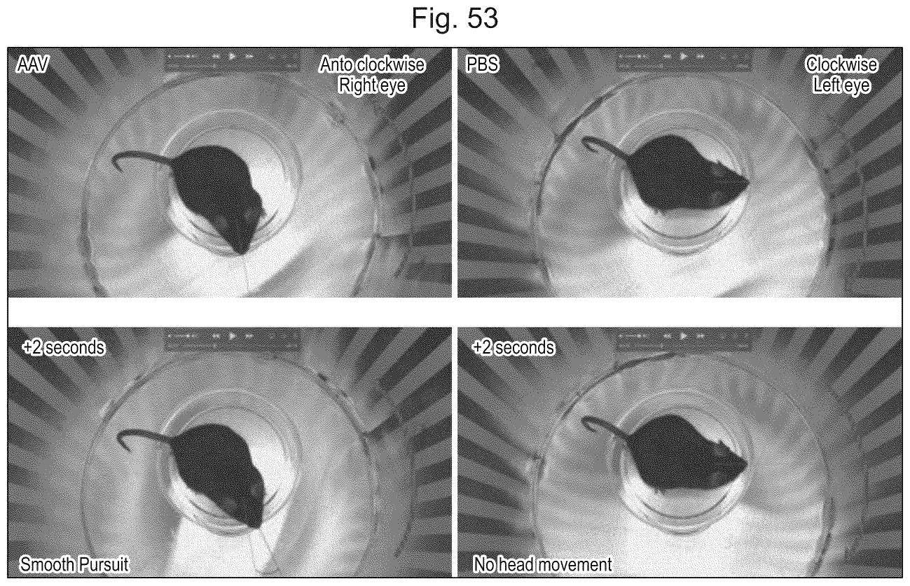

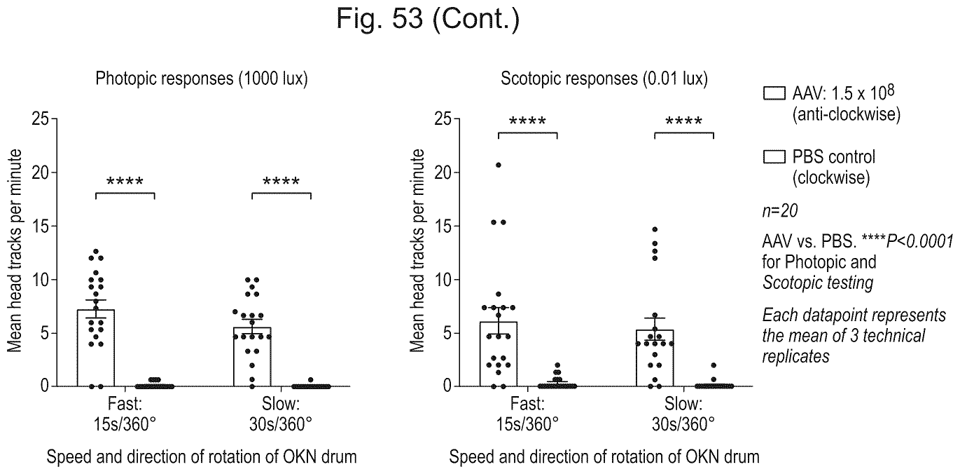

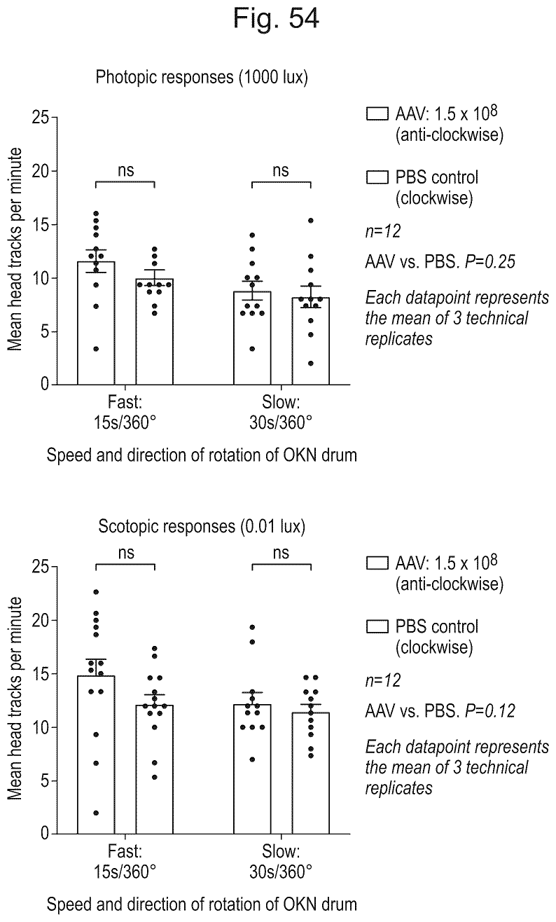

Natural history data are presented identifying the extinction of photopic and scotopic OMR in Cdhr1−/− mice at 20-24 months of age, whilst responses remained identifiable in age-matched C57BL/6J controls. Optomotor responses were preserved in AAV-injected Cdhr1−/− eyes (n=20) under photopic conditions (1000 lux) at 19-months, and scotopic conditions (0.01 lux) at 21-months post-injection at two speeds of rotation of the OKN drum; P<0.0001 for AAV-versus PBS-injected eyes for all speeds and at both levels of illuminance (FIG. 53). The best responders demonstrated optomotor responses that were comparable to C57BL/6J mice tested under the same conditions (FIG. 54).

### Example 10—High-Dose AAV8.GRK1.CDHR1.pA Does Not Affect Photopic and Scotopic Optomotor Reflex Responses in C57BL/6J Mice to 22-Months Post-Injection

Optomotor testing under photopic (20-months) and scotopic (22-months post-injection) conditions did not identify a difference between AAV- and PBS-injected eyes in C57BL/6J mice at either 15 s or 30 s per rotation of the OKN drum (FIG. 54); P=0.25—photopic; P=0.12—scotopic OMR testing

### Example 11—Correlation of Structural and Functional Rescue Following CDHR1 Gene Therapy

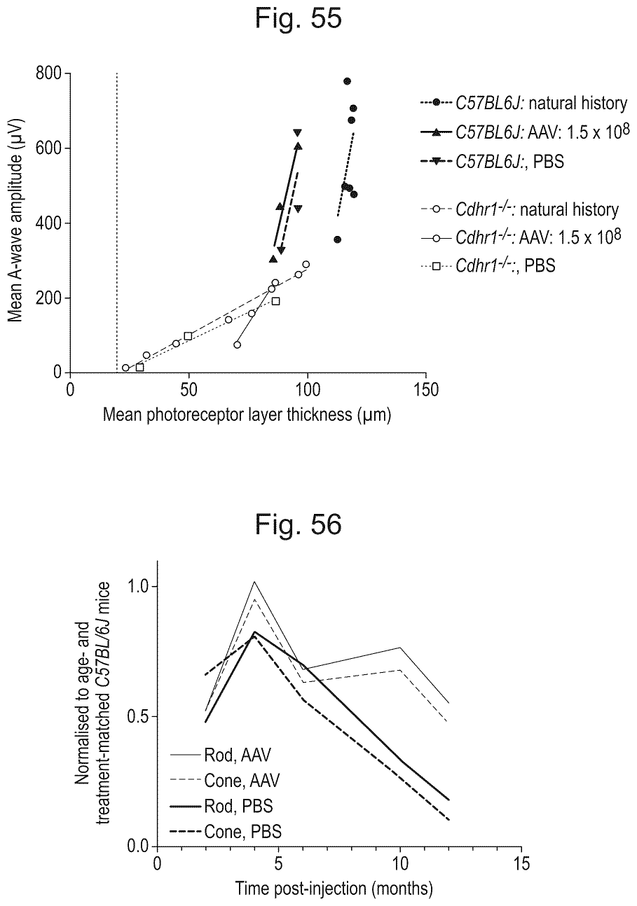

In Cdhr1−/− mice, AAV-injection modified the relationship between structural and functional measures seen in PBS-injected and untreated control eyes, further supporting a slowing of photoreceptor degeneration (R2>0.98 for all groups, FIG. 55).

In C57BL/6J mice, high dose AAV8.GRK1.CDHR1.pA injection did not affect the structure-function correlation observed in PBS-injected and untreated control eyes, further supporting other observations of safety of CDHR1 gene therapy.

### Example 12—Matched Rod and Cone Photoreceptor Rescue Following CDHR1 Gene Therapy

Functional rescue of rod and cone photoreceptor responses were well matched in AAV-injected eyes relative to age- and treatment-matched C57BL/6J mice (FIG. 56), differences also apparent in PBS-injected eyes.

### Example 13—CDHR1 Gene Therapy Prevents Photoreceptor Cell Death in Cdhr1^{-/-} Mice

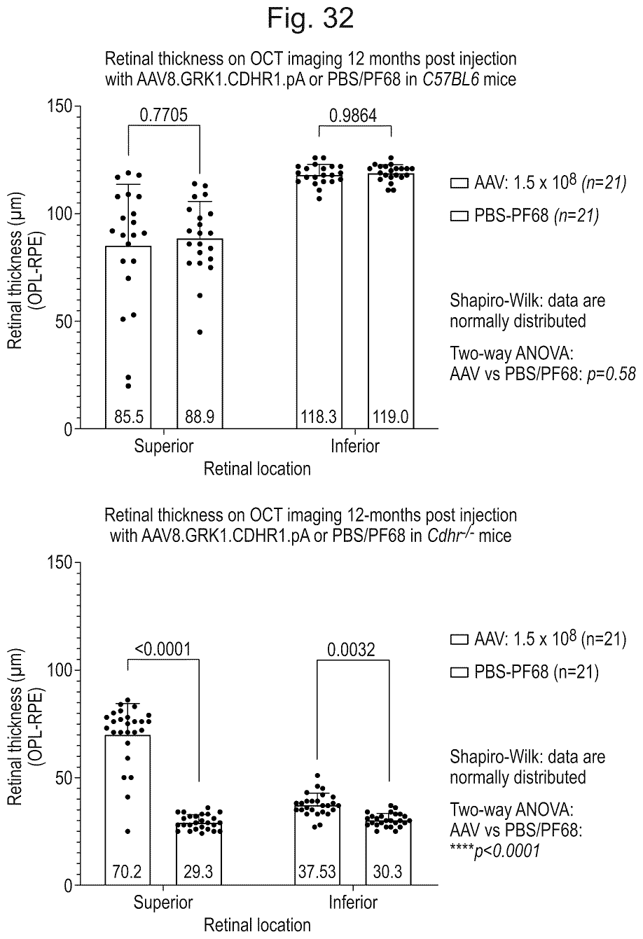

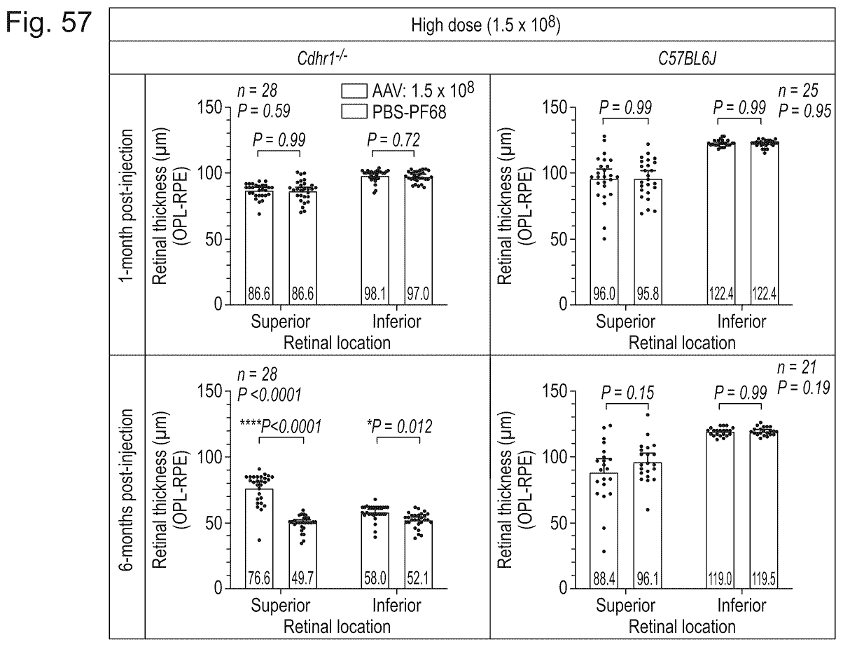

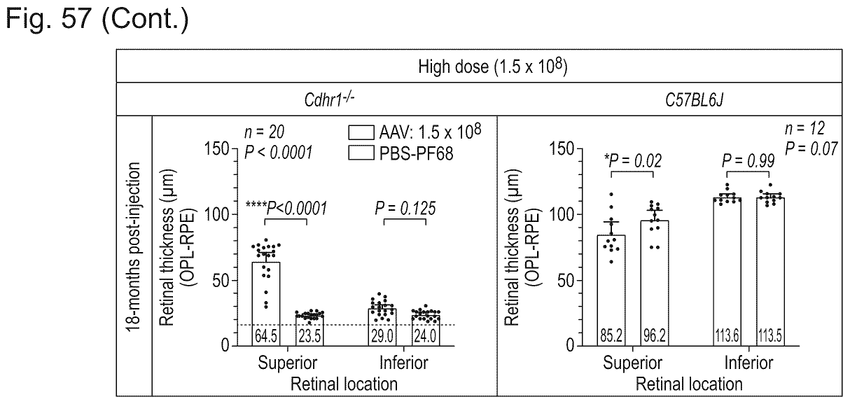

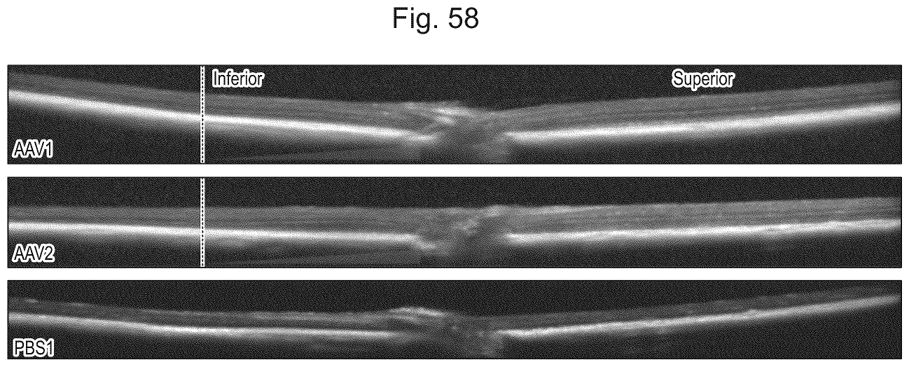

Retinal thickness measurements undertaken 4-weeks post-injection did not reveal differences between high-dose AAV- (1.5×10⁸ gc) and PBS-injected eyes at equivalent superior retinal loci (FIG. 30). However, at 6-months, eyes injected with AAV at a dose of 1.5×10⁸ gc were significantly thicker than paired PBS-injected control eyes (mean 76.6 μm versus 49.7 μm; P<0.0001, two-way ANOVA; FIG. 31). At 12-months, the difference in superior retinal thickness had increased to a mean of 70.2 μm versus 29.3 μm (P<0.0001, two-way ANOVA; FIG. 32). At 18-months, the difference had increased to 64.5 μm versus 23.5 μm (P<0.0001; n=20), with further thinning in PBS-injected eyes limited by a floor effect (FIGS. 57 and 58).

At 1.5×10⁸ gc, inferior retinal locations which were presumed not to be detached as part of the surgical procedure were also significantly thicker in AAV- versus PBS-injected control eyes at 6-months (58.0 μm vs 52.1 μm; P=0.012). This effect remained statistically significant at 12-months although with a smaller effect size (37.5 μm vs 30.3 μm; n=21; P=0.0032) and not significant at 18 months post-injection (P=0.125; n=20). However, retinal structure was preserved in the superior half of the inferior retina (FIG. 58), when compared to the peripheral inferior retinal loci where OCT measurements were derived. This is a likely consequence of a superior-inferior gradient of transduction at 1.5×10⁸ vg, beyond the limits of the initial bleb. Further analysis was undertaken at 6- and 12-months on nasal and temporal retinal locations since they were not subject to surgical trauma and received a greater dose than inferior retinal locations. At 12-months, AAV-injected eyes were significantly thicker than PBS-injected eyes at nasal and temporal retinal locations (P<0.0001 for both locations; two-way ANOVA; data not shown).

At the low dose of 1.5×10⁷ gc, retinal thickness measurements at superior retinal locations were not significantly different at 4-weeks post-injection (mean 75.9 μm versus 79.4 μm; P=0.18, two-way ANOVA; FIG. 30). However, AAV-injected eyes were less thinned at 6-months (mean 49.7 μm versus 45.3 μm; P=0.028, two-way ANOVA) at superior retinal locations. There was no significant difference in inferior retinal thickness measurements at 6-months (P=0.66). Raw OCT images illustrate the structural rescue effect of high dose AAV8.GRK1.CDHR1.pA (1.5×10⁸ vg) on the preservation of the photoreceptor layer in AAV- versus PBS-injected control eyes (FIGS. 58 to 61). This effect is sustained to at least 18-months after intervention, the latest timepoint measured.

### Example 14—CDHR1 Gene Therapy Regenerates Photoreceptor Outer Segments

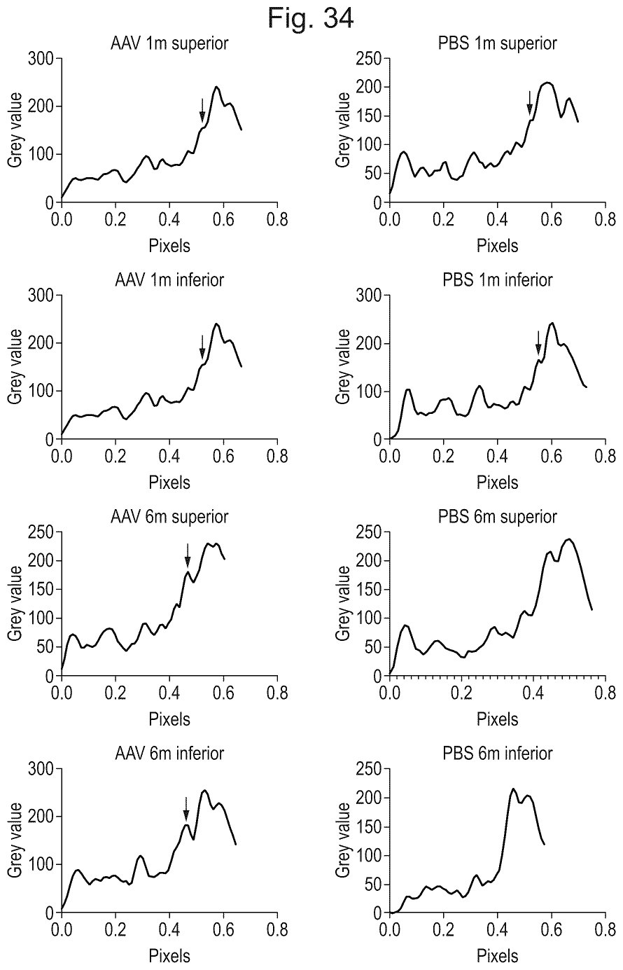

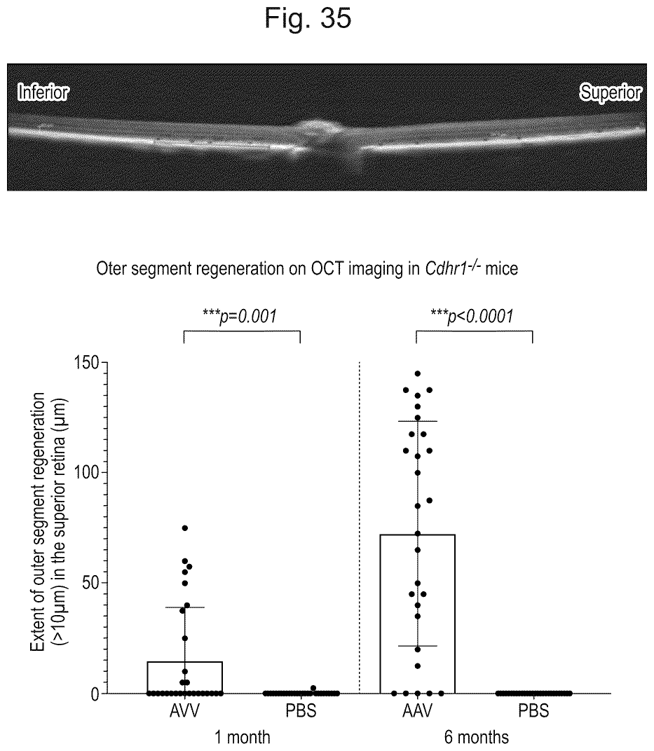

In addition to preservation of the outer retina, as defined as the distance between the outer plexiform layer and retinal pigment epithelium, several morphological changes were evident in Cdhr1^{-/-} mice following high-dose (1.5×10⁸ gc) AAV8.GRK1.CDHR1.pA. As early as 1-month after subretinal AAV injection, 11 of 28 mice showed evidence of lengthening of the photoreceptor outer segment band on OCT imaging (FIG. 35). By 6-months, 23 of 28 mice in this group exhibited this morphological change, which was not evident in PBS-injected control eyes at any timepoint (FIG. 35). Measurement of the extent of outer segment lengthening from a single vertical OCT section through the optic disc demonstrated a mean extent of outer segment lengthening of 2900 μm at 6 months (FIG. 35). Outer segment lengthening was not seen in the low-dose AAV group at 6 months. Furthermore, the ellipsoid band representing the boundary of inner and outer segments of photoreceptors appeared both thicker and more hyper-reflective in AAV-injected eyes at 6 months and more so at 12-months post-injection (FIGS. 33 & 34). This was evident as early as 1-month although was more prominent in extent at 6- and 12-months, as shown on OCT reflectivity profiles in FIG. 34. The external limiting membrane, which is no longer discernible in Cdhr1^{-/-} mice at 6 months of age was present at 6-months in eyes injected with high-dose AAV as shown in FIG. 34 at superior retinal locations.

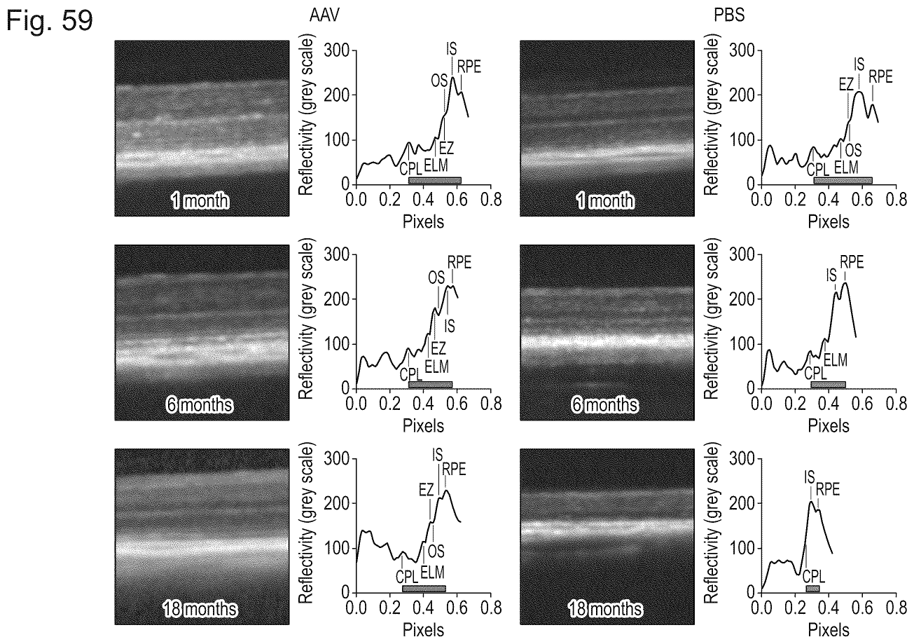

High-dose AAV.GRK1.CDHR1.pA increased the reflectivity of the ellipsoid zone and preserved the external limiting membrane in Cdhr1−/− mice to 18-months post-injection. In Cdhr1−/− mice, the EZ is no longer discernible on OCT imaging after 6-months of age. Moreover, when present, the EZ appears less reflective compared to age-matched wildtype controls, appearing similar in width and reflectivity to the ELM (FIGS. 5, 59 and 61). High-dose AAV8.GRK1.CDHR1.pA preserved the EZ to 18-months post-injection in Cdhr1−/− mice, associated with an increase in EZ reflectivity and broadening of the EZ band over time (FIGS. 59 to 61). In the inferior peripheral retina, the EZ is lost with continued retinal degeneration at later timepoints (FIG. 58). Transmission electron microscopy confirmed the number and morphology of mitochondria in the inner segments of AAV-treated Cdhr1−/− eyes were as per wildtype controls—the anatomical correlate for the EZ on OCT imaging (FIG. 68). Moreover, the ELM, which is normally lost by 12-months of age in Cdhr1−/− mice is preserved to at least 18-months following high-dose AAV8.GRK1.CDHR1.pA in AAV-injected eyes (FIGS. 59 to 61). The ELM does not appear to increase in intensity at later timepoints.

There were no specific morphological alterations seen in C57BL6J mice in either AAV-dose group.

Following high dose AAV8.GRK1.CDHR1.pA injection (1.5×10⁸ vg), preservation and regeneration of the OCT layer representing the photoreceptor OS was identified in AAV-injected eyes to 18-months post-injection on OCT imaging (FIGS. 58 to 61). These effects were not identified in the low-dose cohort, or PBS-injected eyes.

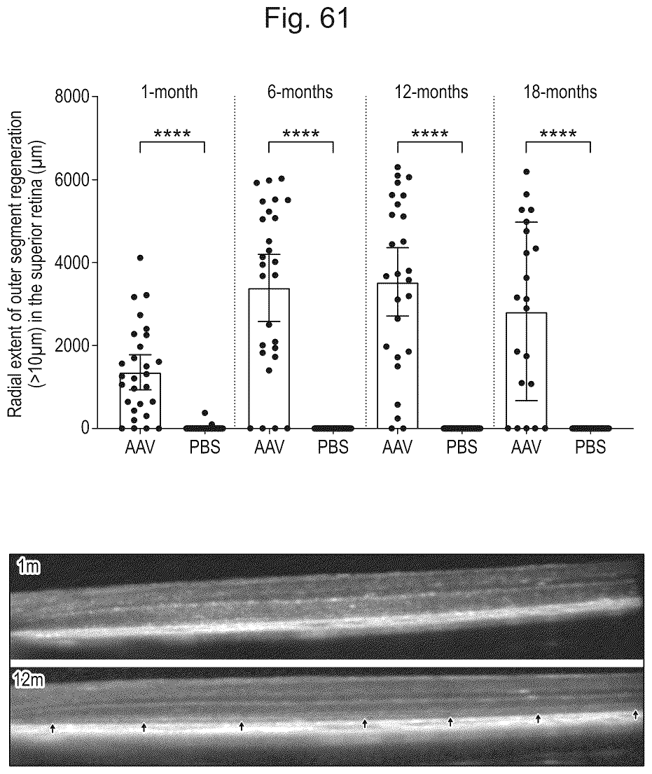

High-dose AAV8.GRK1.CDHR1.pA was found to lengthen the layer representing photoreceptor OS on OCT imaging in Cdhr1−/− mice as early as 1-month post-injection, which increased in radial extent across the superior retina to 18-months post-injection (P<0.0001 at all timepoints vs. PBS-injected eyes; FIG. 61). OCT evidence of OS lengthening of the superior half of the inferior retina was observed (shown in FIG. 58) in some AAV-injected eyes yielding a significant overall benefit compared with PBS-injected control eyes, although the effect size was smaller.

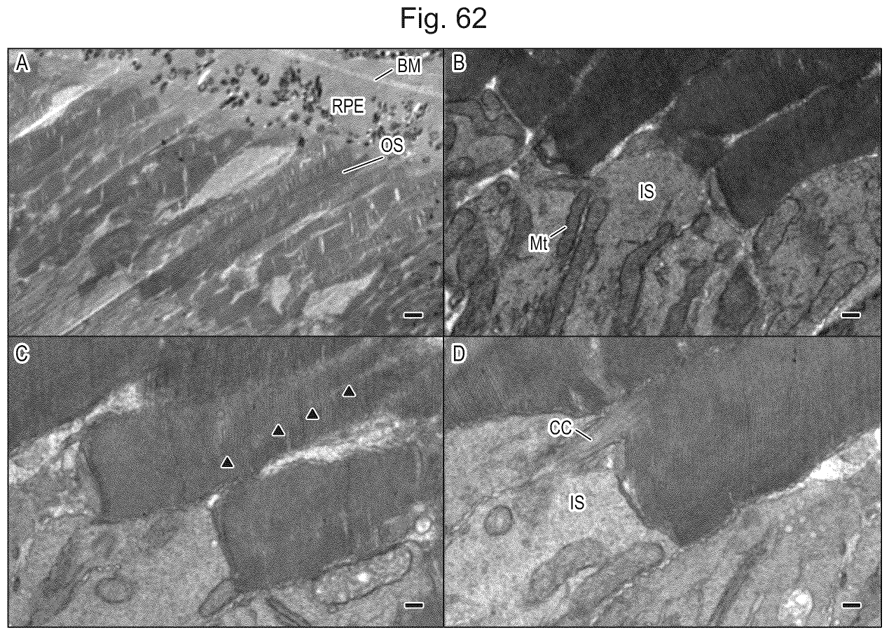

Transmission electron microscopy confirmed OS regeneration following high dose AAV8.GRK1.CDHR1.pA in Cdhr1−/− eyes to 21-months post-injection (FIG. 62). OS measured approximately 30 μm, similar to wildtype, with regular alignment, orientation and disc spacing. Incisures in rod outer segments were identifiable with normal morphology of nascent outer segment discs along the periciliary ridge of the inner segment. Photoreceptor and outer segments were not identifiable in paired PBS-injected control eyes.

### Example 15—High-Dose AAV.GRK1.CDHR1.pA Prevents RPE Degeneration to 18-Months Post-Injection in Cdhr1−/− Mice

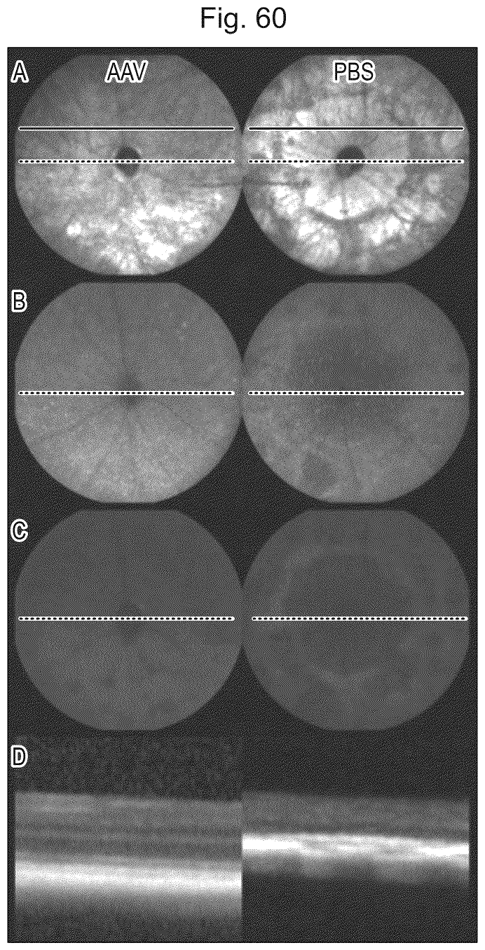

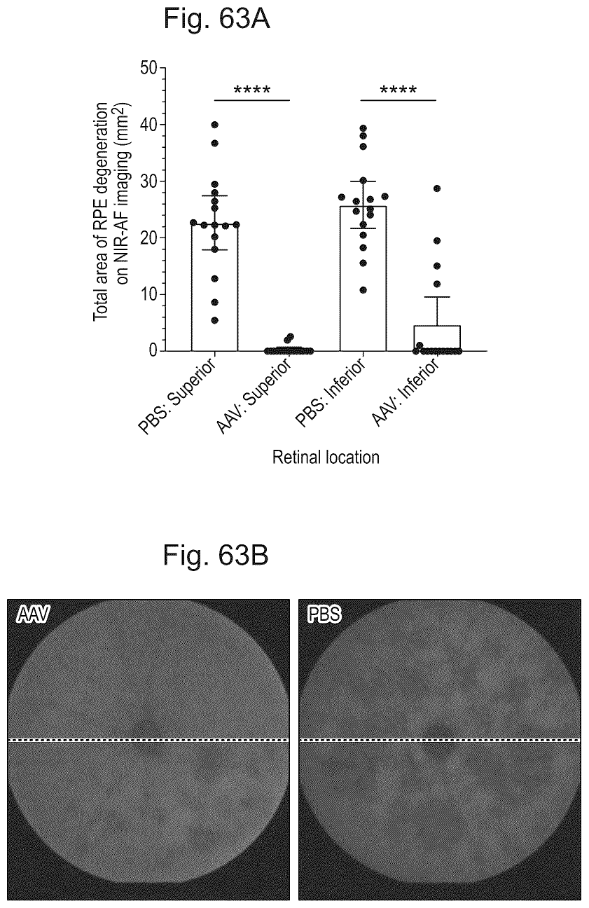

In PBS-injected Cdhr1−/− mice, progressive, patchy RPE atrophy was demonstrated, most evident on NIR-AF imaging at 18-months post-injection (FIGS. 60 and 63). High dose AAV8.GRK1.CDHR1.pA prevented RPE degeneration on NIR-AF imaging compared to PBS-injected eyes (P<0.0001) at 18-months post-injection. Moreover, this effect was greater in treated superior retinal locations, although a benefit was also identified inferiorly in AAV-injected eyes (P<0.0001 for both retinal locations versus PBS-injected eyes; FIG. 51). Contiguous areas of RPE atrophy were not present at the 6-month timepoint in PBS-injected eyes, and was therefore not assessed in the low dose groups.

### Discussion

The present Examples demonstrate the first therapy shown to ameliorate progressive photoreceptor degeneration in a validated model of CDHR1-associated retinal degeneration. In Cdhr1^{-/-} mice, sub-retinal delivery of AAV8.CDHR1 rescued A-wave amplitudes (p<0.0001 at all time points) and B-wave amplitudes (p<0.0001 from 6 months) on dark-adapted electroretinography when compared with PBS-injected control eyes. Light-adapted flicker ERG amplitudes were greater in AAV-treated eyes at 10-months post-injection (p<0.0001). Retinal imaging findings consistent with regeneration of the photoreceptor outer segment were only identified in AAV-treated eyes, with therapeutic effect seen as early as 1-month post-injection (p=0.001). CDHR1 gene therapy reduced the rate of photoreceptor cell death as indicated photoreceptor layer thickness measurements compared to controls at 6-months post-injection (p<0.0001). Sub-retinal delivery of AAV.CDHR1 was safe in C57BL6J mice as evaluated by structural and functional measures. AAV-mediated expression of the human CDHR1 transgene in the Cdhr1^{-/-} murine retina rescued cone and rod photoreceptor function through restoration of photoreceptor outer segment band and slowing of photoreceptor cell death. These beneficial structural effects were mostly limited to the superior retina of AAV-treated eyes, although mild inferior structural preservation and outer segment regeneration was attributable to a reduced level of CDHR1 expression shown in the inferior retina.

AAV8.GRK1.CDHR1.pA delivered by sub-retinal injection at a dose of 1.5×10⁸ vg preserved rod- and cone-photoreceptor response amplitudes and reduced light- and dark-adapted photoreceptor response implicit times on electroretinography to at least 12-months post-injection in Cdhr1−/− mice when compared to PBS-injected control eyes. A slowing of photoreceptor cell death, complete regeneration of rod photoreceptor outer segment length, alignment and morphology on electron microscopy, increased reflectivity of the ellipsoid zone and preservation of the ELM on OCT imaging were seen only in AAV-injected eyes, with the treatment effect sustained to at least 21-months post-injection. A clear benefit of high-dose AAV8.GRK1.CDHR1.pA on visual behaviour was demonstrated on photopic and scotopic optomotor testing, whilst responses were absent in PBS-injected eyes at 19-21 months post-injection—the most durable therapeutic response of any pre-clinical murine study of AAV retinal gene therapy. Longitudinal structural, functional and behavioural testing, using identical experimental protocols, did not detect any significant toxic effects of AAV8.GRK1.CDHR1.pA at 1.5×10⁸ vg in C57BL/6J mice to 22-months post-injection when compared to PBS-injected control eyes, except for very mild thinning on OCT imaging in matched superior retinal locations measured at 18-months post-injection. A dose of 1.5×107 vg was neither significantly therapeutic in Cdhr1−/− mice, nor toxic in C57BL/6J mice to 6-months post-injection.

Hence, a single sub-retinal injection of 1.5×10⁸ vg of AAV.GRK1.CDHR1.pA delivered at 3-4 weeks of age preserved rod- and cone-photoreceptor response amplitudes, reduced rod- and cone photoreceptor response implicit times, slowed photoreceptor cell death, preserved outer retinal structures (e.g. ELM and EZ on OCT imaging) and regenerated full-length, morphologically normal photoreceptor outer segments at 22-months post-injection in AAV-injected Cdhr1−/− eyes. A clear beneficial effect on visual behaviour through photopic and scotopic optomotor testing was demonstrated in AAV-injected eyes at 19-21 months post-injection. The treatment benefit persisted to at least 21-months post-injection, the most durable response following AAV-gene therapy demonstrated in any pre-clinical murine model of retinal degeneration to date.

PBS-injected Cdhr1−/− control eyes exhibited severe outer retinal degeneration without identifiable OS, and with minimal or absent functional responses at the equivalent timepoints. Evidence of a true therapeutic effect was further supported by the dose-dependent and location-dependent nature of the observed therapeutic effects (i.e. using the inferior retina as an internal control) and incremental therapeutic effects on both longitudinal structural and functional analyses, with a greater therapeutic effect size demonstrated across four timepoints on OCT imaging and six timepoints on dark- and light-adapted ERG.

CDHR1 functions in the development of nascent photoreceptor outer segment discs and the higher order organisation of the outer segment. Restoration of the CDHR1 transgene to rod and cone photoreceptors may result in progressive regeneration of photoreceptor outer segments as demonstrated on OCT imaging and confirmed on electron microscopy in this study. Moreover, this beneficial morphological correction appears to begin as early as 1-month, increases to 12-months, and persists to at least 21-months post-injection. Late OS regeneration (i.e. seen at 6-months onwards) may reflect the time required for photoreceptor transduction, a process which has been shown to further increase between 6- and 12-months post-injection in the murine retina. Moreover, the process of outer segment renewal occurs at a rate of approximately 2.3 μm per day with the outer segment measuring approximately ˜20-25 μm in C57BL/6J mice. Cadherins form a superfamily of proteins characterised by presence of two or more extracellular cadherin repeats. Through homophilic or heterophilic interactions, cadherins serve crucial roles in cell-to-cell adhesion. In highly evolved sensory cells, such as the photoreceptors in the retina and the hair cells of the inner ear, cadherins serve more complex functions, principally through heterophilic interactions with other proteins to support the function of the cilia. To our knowledge, this is the first time that a transgene expressing a cadherin has been shown to ameliorate a disease phenotype in vivo.

The observation of outer segment regeneration following AAV8.CDHR1 gene therapy suggests successful heterophilic interaction of CDHR1 with an as yet unidentified binding partner within the periciliary ridge of the inner segment. Moreover, lengthening of the outer segments following gene therapy suggests successful uncoupling of CDHR1-based connections to the inner segment following horizontal outer segment disc elongation. This further suggests successful post-translational modification of the expressed human CDHR1 protein.

### Example 16—Retinal Phenotype of Human Subjects with Bi-Allelic Variants in CDHR1

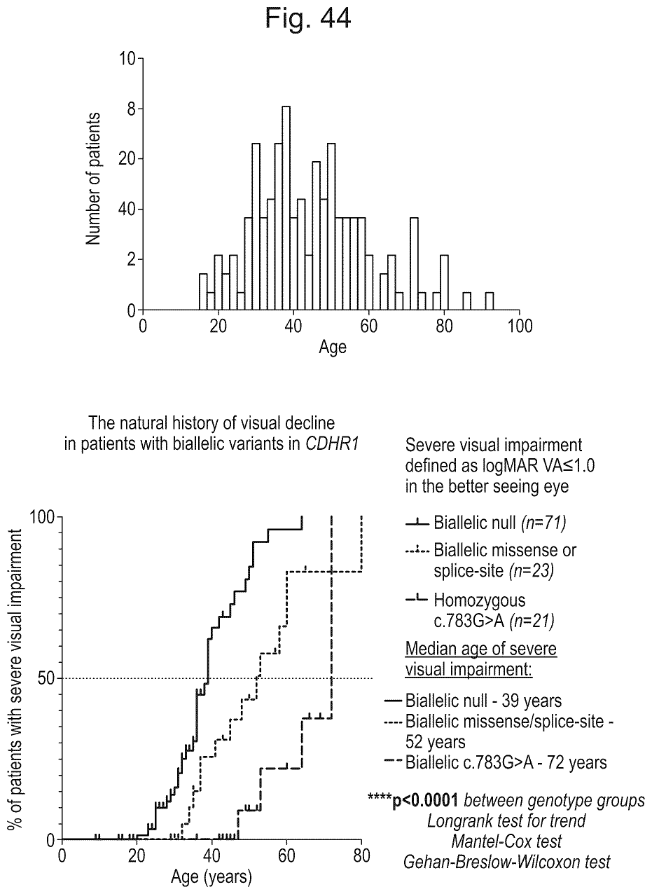

A better characterisation of the natural history of CDHR1-related retinal degeneration will help to determine the most appropriate timing, and support the ethical approval for intervention in a given patient. Original data collected involving an international collaboration identified 149 individuals with retinal degeneration due to biallelic CDHR1 variants. Furthermore, the study has established key aspects of the natural history of the disease. All individuals with biallelic null variants in CDHR1, as modelled by the Cdhr1^{-/-} mouse are blind by the age of 60 (FIG. 44). Moreover, many of the patients in this study are younger than 60 (as seen in FIG. 44), and intervention in this group may prevent blindness. This finding highlights the need for a therapeutic for this patient group. The study has further identified the main retinal phenotypes associated with biallelic CDHR1 variants: cone-rod dystrophy, retinitis pigmentosa and late-onset macular dystrophy, the latter of which is associated with hypomorphic (partially functional) variants. The most common, known hypomorphic allele is the c.783G>A variant.

### Example 17—CDHR1-Associated Retinal Degeneration is Estimated to Affect >200,000 Individuals Worldwide

Using a published methodology that uses the Hardy-Weinberg equilibrium to calculate the carrier frequency and genetic prevalence of disease (PMID: 31964843), we calculated the global genetic prevalence of CDHR1-associated retinal degeneration using all validated disease-associated variants (including ˜60 novel variants identified in the international collaborative study. We calculated a genetic prevalence estimate for all six major characterised world populations (European, European Finnish, African, South Asian, East Asian, Latino) based on the allele frequency of all reported pathological CDHR1 variants in that population.

This calculation predicts more than 200,000 individuals worldwide who are expected to manifest or to later develop retinal degeneration due to CDHR1 variants, as shown in Table 2.

TABLE 2
All pathological CDHR1 alleles

| | | Affected | Heterozygous |
|---|---|---|---|
| | Population | individuals | carriers |
| UK | 66,650,000 | 3,189 | 915,638 |
| North America | 574,000,000 | 27,462 | 7,885,614 |
| Europeans | 735,802,007 | 35,203 | 10,108,451 |
| European - Finnish | 5,516,176 | 200 | 66,075 |
| Africans | 1,258,311,831 | 18,433 | 9,595,219 |
| Latinos | 646,059,776 | 2,716 | 2,643,942 |
| South Asians | 1,870,824,410 | 8,367 | 7,895,919 |
| East Asians | 1,646,834,954 | 119,451 | 27,812,245 |
| WORLDWIDE | 6,737,349,154 | 211,832 | 66,007,465 |
| | 1 in every | 31,805 | 102 |
| Europe & North | 1,309,802,007 | 62,665 | 17,994,065 |
| America only | | | |
| | 1 in every | 20,902 | 73 |

In Europe and North America, an estimated 62,665 individuals are expected to be affected (˜1 in 20,902 individuals), with a carrier frequency of 1 in 73.

Furthermore, by establishing the retinal phenotype associated with each variant, we were able to calculate the following genetic prevalence estimates by retinal phenotype, as shown in Tables 3 and 4.

### Tables 3 and 4

| | | CDHR1 macular | CDHR1 |
|---|---|---|---|
| | | dystrophy | RP/CRD |
| UK | 66,500,000 | 3,031 | 150 |
| North America | 574,000,000 | 26,164 | 1,298 |
| Europeans | 735,802,007 | 33,539 | 1,554 |
| European - Finnish | 5,516,176 | 200 | 0 |
| Africans | 1,258,311,831 | 12,206 | 6,226 |
| Latinos | 646,059,776 | 1,399 | 1,317 |
| South Asians | 1,870,824,410 | 4,593 | 3,814 |
| East Asians | 1,646,834,954 | 118,228 | 1,223 |
| WORLDWIDE | 6,737,349,154 | 196,289 | 15,693 |
| | Global genetic prevalence of CDHR1-associated | | |
| | late onset macular dystrophy | | |
| | 1 in every | 34,324 | 429,311 |
| Europe & North | 1,315,318,183 | 59,903 | 2,962 |
| America | | | |
| | 1 in every | 21,958 | 444,025 |

| Carriers of a | Carriers of a |
|---|---|
| null variant | hypomorphic variant |
| 198,627 | 714,950 |
| 1,714,467 | 6,171,148 |
| 2,197,749 | 7,910,702 |
| 1,475 | 64,600 |
| 5,576,687 | 4,018,532 |
| 1,841,092 | 802,850 |
| 5,331,141 | 2,564,778 |
| 2,814,521 | 24,997,724 |
| 19,477,132 | 46,530,333 |
| 1 in every | |
| 346 | 145 |

TABLE 5
Clinical features of 10 patients with CDHR1-associated retinopathy.

| | | | | | | | |
|---|---|---|---|---|---|---|---|
| | | First | | | | | |
| Patient # | | symptoms | | | | | |
| (age at last | | (age at first | | Best | Refraction | | |
| examination | CDHR1 | symptoms | Current | corrected | (spheric/ | | |
| [years]) | mutation | [years]) | symptoms | visual acuity | cylinder) | Visual field | ERG |

Group 1: c.783G > A - homozygous

| | | | | | | | |
|---|---|---|---|---|---|---|---|
| 1.1 (46) | c.783G > A | Metamorphopsia | Metamorphopsia, | OD 20/20 | OD −4.0/−1.25 | Humphrey 30°: | Borderline low |
| | homozygous | (45) | NO night vision | OS 20/20 | OS −3.0/−1.0 | relative | photopic responses, |
| | | | problems | | | paracentral | scotopic responses |
| | | | | | | scotoma | within normal limits |
| 1.2 (45) | | Reading | Mild patchy vision | OD 20/20 | Not assessed | N.P. | Photopic and |
| | | difficulties | OD | OS 20/20 | | | scotopic responses |
| | | (40) | | | | | within normal limits |
| 1.3 (49) | | Glare | Glare, reading | OD 20/25 | OD −6.25/−1.0 | Goldmann III/4 | Photopic > scotopic |
| | | (42) | difficulties, NO | OS 20/25 | OS −6.0/−1.5 | normal peripheral | responses mildly |
| | | | night vision | | | thresholds | reduced |
| | | | problems | | | | |
| 1.4 (51) | | Metamorphopsia, | Reduced visual | OD 20/200 | OD −4.0/−1.0 | Microperimetry: | — |
| | | reading | acuity, glare, NO | OS 20/200 | OS −2.25/−0.75 | sensitivity outside | |
| | | difficulties | night vision | | | atrophy normal | |
| | | (41) | problems | | | | |
| 1.5 (63) | | Metamorphopsia | Reduced visual | OD CF | OD −6.25/−1.0 | N.P. | Photopic and |
| | | (43) | acuity, glare, poor | OS 20/1000 | OS −5.75/−0.75 | | scotopic responses |
| | | | contrast. NO | | | | within normal limits |
| | | | night vision | | | | |
| | | | problems | | | | |
| 1.6 (76) | | ? | Reduced visual | OD 20/800 | OD +1.75/−0.75 | Goldmann III/4 | Photopic and |
| | | (55) | acuity, glare, NO | OS 20/50 | OS +2.5/−0.25 | normal peripheral | scotopic responses |
| | | | night vision | | | thresholds | within normal limits |
| | | | problems | | | | |

Group 2: c.783G > A - compound heterozygous with different second CDHR1 mutation

| | | | | | | | |
|---|---|---|---|---|---|---|---|
| 2.1 (52) | c.783G > A | Glare | Reduced visual | OD 20/200 | OD −0.75 | Microperimetry: | Photopic~scotopic |
| | c.2522_2528del | (47) | acuity, glare, | OS 20/200 | OS −0.5/−0.25 | sensitivity outside | responses reduced |
| | | | night vision | | | atrophy reduced | |
| | | | problems | | | | |
| 2.2 (54) | c.783G > A | Glare | Reduced visual | OD 20/50 | OD −2.0/−0.25 | N.P. | Photopic responses |
| | c.1570_1592del | (45) | acuity, glare, poor | OS 20/80 | OS −2.5/−1.5 | | reduced, scotopic |
| | | | reading ability, | | | | responses within |
| | | | reduced color | | | | normal limits |
| | | | vision | | | | |

Group 3: 2 different CDHR1 mutations

| | | | | | | | |
|---|---|---|---|---|---|---|---|
| 3.1 (49) | c.1503_1507del | Glare, | Reduced visual | OD 20/800 | OD +2.5/−4.0 | Microperimetry: | Photopic and |
| | homozygous | reduced | acuity, glare, | OS 20/800 | OS +2.5/−4.75 | sensitivity outside | scotopic responses |
| | | visual | night vision | | | atrophy severely | extinguished |
| | | acuity (43) | problems | | | reduced | |
| 3.2 (39) | c.18G > A | Nyctalopia | Nyctalopia, glare, | OD 20/600 | N.P. (bilateral | Concentric | Photopic and |
| | c.438 + 1G > A | (~25) | reduced visual | OS CF | corneal graft for | restriction | scotopic responses |
| | | | acuity | | keratoconus | | extinguished |

Note the absence of nyctalopia in patients from group 1 homozygous for the CDHR1 c.783G > A variant which are present in group 2 and most significantly in group 3. Abbreviations: OCT, optical coherence tomography; ERG, electroretinography; CF, counting fingers; NP, not performed.

Patients in group 1 are homozygous for c.783G>A variant, patients in group 2 have one c.783G>A variant and one truncating variant, and patients in group 3 have two truncating variants in CDHR1. In retinal imaging using fundus autofluorescence (488 nm), near infra-red autofluorescence (790 nm), SD-OCT central, well-defined area of retinal atrophy can be seen in most patients except for patient 1 who has an early stage of disease. Group 1 patients have qualitatively normal fundus autofluorescence and OCT imaging outside the central area of atrophy. Group 2 patients have some evidence of structural abnormalities outside of the area of macular atrophy. Group 3 patients have significant peripheral retinal degeneration with RPE loss. SD-OCT imaging demonstrates interruption of the EZ in patient 1.1 (early disease) and IZ with eventual loss of these layers and the RPE in the central zone with ONL thinning. Patient 2.2 has generalised thinning of the ONL beyond the central area. Patients in group 3 have generalised outer retinal thinning beyond the central area of macular atrophy.

This series in particular highlights the relatively high frequency of patients with the hypomorphic variant (c.783G>A) in CDHR1. This variant leads to in-frame exon skipping an unusual phenotype with early macular involvement without peripheral degeneration. Patients in this group develop visual symptoms in their 5^{th} decade which progresses to severe loss of central vision within 10 years. However, patients with two null variants in CDHR1 present earlier with more severe visual loss affecting the central and peripheral retina on functional testing as characterised on visual field testing and ERG. Patients with one c.783G>A variant and one null variant have an intermediate phenotype. This data suggests a wide-therapeutic window for intervention with gene replacement during which time the natural history of disease may be ameliorated.

Sequences

SEQ ID NO 1-DNA sequence for AAV2 ITR (5′)
TAGCTGCGCGCTCGCTCGCTCACTGAGGCCGCCCGGGCAAAGCCCGGGCGTCGGGCGACCTTTGGTCGCCCGGCCTCAGTGAGCGAGCGAG
CGCGCAGAGAGGGAGTGGCCAACTCCATCACTAGGGGTTCCT
SEQ ID NO 2-Human rhodopsin kinase promoter (GRK1)
GGGCCCCAGAAGCCTGGTGGTTGTTTGTCCTTCTCAGGGGAAAAGTGAGGCGGCCCCTTGGAGGAAGGGGCCGGGCAGAATGATCTAATCG
GATTCCAAGCAGCTCAGGGGATTGTCTTTTTCTAGCACCTTCTTGCCACTCCTAAGCGTCCTCCGTGACCCCGGCTGGGATTTAGCCTGGT
GCTGTGTCAGCCCCGGG
SEQ ID NO 3-Human rhodopsin promoter (RHOp)
CAGATCTTCCCCACCTAGCCACCTGGCAAACTGCTCCTTCTCTCAAAGGCCCAAACATGGCCTCCCAGACTGCAACCCCCAGGCAGTCAGG
CCCTGTCTCCACAACCTCACAGCCACCCTGGACGGAATCTGCTTCTTCCCACATTTGAGTCCTCCTCAGCCCCTGAGCTCCTCTGGGCAGG
GCTGTTTCTTTCCATCTTTGTATTCCCAGGGGCCTGCAAATAAATGTTTAATGAACGAACAAGAGAGTGAATTCCAATTCCATGCAACAAG
GATTGGGCTCCTGGGCCCTAGGCTATGTGTCTGGCACCAGAAACGGAAGCTGCAGGTTGCAGCCCCTGCCCTCATGGAGCTCCTCCTGTCA
GAGGAGTGTGGGGACTGGATGACTCCAGAGGTAACTTGTGGGGGAACGAACAGGTAAGGGGCTGTGTGACGAGATGAGAGACTGGGAGAAT
AAACCAGAAAGTCTCTAGCTGTCCAGAGGACATAGCACAGAGGCCCATGGTCCCTATTTCAAACCCAGGCCACCAGACTGAGCTGGGACCT
TGGGACAGACAAGTCATGCAGAAGTTAGGGGACCTTCTCCTCCCTTTTCCTGGATCCTGAGTACCTCTCCTCCCTGACCTCAGGCTTCCTC
CTAGTGTCACCTTGGCCCCTCTTAGAAGCCAATTAGGCCCTCAGTTTCTGCAGCGGGGATTAATATGATTATGAACACCCCCAATCTCCCA
GATGCTGATTCAGCCAGGAGCTTAGGAGGGGGAGGTCACTTTATAAGGGTCTGGGGGGGTCAGAACCCAGAGTCATCC
SEQ ID NO 4-Chicken beta-actin promoter exon-intron-exon (Ex/In/Ex)
TCGCTGCGCGCTGCCTTCGCCCCGTGCCCCGCTCCGCCGCCGCCTCGCGCCGCCCGCCCCGGCTCTGACTGACCGCGTTACTCCCACAGGT
GAGCGGGCGGGACGGCCCTTCTCCTCCGGGCTGTAATTAGCGCTTGGTTTAATGACGGCTTGTTTCTTTTCTGTGGCTGCGTGAAAGCCTT
GAGGGGCTCCGGGAGGGCCCTTTGTGCGGGGGGAGCGGCTCGGGGCTGTCCGCGGGGGGACGGCTGCCTTCGGGGGGGACGGGGCAGGGCG
GGGTTCGGCTTCTGGCGTGTGACCGGCGGCTCTAGAGCCTCTGCTAACCATGTTCATGCCTTCTTCTTTTTCCTACAGCTCCTGGGCAACG
TGCTGGTTATTGTGCTGTCTCATCATTTTGGCAAAGAATTGGATCCTAGCTTGATATCGAATTCCTGCAG
SEQ ID NO 5-Reference DNA sequence for CDHR1
ATGAGGCGCTGCCGGTGGGCCGCCCTGGCCCTGGGGCTGCTGCGCCTCTGCTTGGCTCAGGCCAACTTCGCCCCGCACTTCTTCGACAACG
GGGTCGGCAGCACCAACGGAAACATGGCTCTGTTCAGCCTCCCAGAGGACACCCCTGTAGGCTCTCACGTATACACCCTGAATGGGACAGA
CCCTGAGGGAGACCCCATCTCCTACCACATCAGCTTTGACCCCAGCACTAGAAGCGTCTTTTCTGTTGACCCCACTTTTGGAAACATCACC
CTGGTTGAAGAGCTGGACAGAGAGAGGGAAGATGAGATTGAAGCCATCATCAGCATTTCTGATGGCCTGAATCTGGTGGCCGAAAAAGTCG
TGATCCTGGTGACCGATGCCAATGATGAGGCGCCCAGGTTCATCCAGGAGCCTTATGTTGCCCTGGTTCCCGAGGACATACCTGCTGGGAG
CATCATCTTTAAGGTCCATGCAGTGGACAGGGACACAGGCTCTGGAGGGAGTGTCACCTACTTCCTGCAGAACCTGCACTCCCCATTTGCC
GTGGACCGCCACAGCGGTGTGCTGCGCCTCCAGGCTGGGGCCACTCTGGACTACGAGAGGTCCCGGACCCACTACATCACCGTGGTCGCCA
AGGATGGCGGTGGGAGGCTTCATGGGGCTGATGTGGTGTTCTCAGCCACCACCACGGTCACGGTCAATGTGGAGGATGTTCAGGACATGGC
CCCTGTCTTCGTGGGCACACCCTACTATGGCTATGTGTACGAGGACACCCTTCCGGGCTCGGAGGTACTGAAGGTGGTCGCCATGGATGGA
GACCGGGGCAAACCCAATCGAATTCTCTACAGCCTTGTAAATGGGAACGATGGAGCCTTTGAAATTAATGAGACATCTGGAGCCATCTCCA
TCACTCAGAGCCCGGCCCAGCTCCAGAGAGAGGTGTATGAGCTGCATGTACAGGTGACTGAAATGAGCCCTGCGGGGAGCCCAGCTGCCCA
GGCCACCGTCCCAGTCACCATCAGGATTGTGGACCTCAACAACCACCCGCCAACATTCTATGGAGAGAGCGGACCCCAAAACAGGTTTGAG
CTGTCCATGAATGAGCACCCACCCCAGGGAGAGATCCTGCGGGGCCTCAAGATCACCGTCAATGACTCCGACCAGGGAGCCAATGCCAAAT
TCAACTTGCAGCTGGTGGGACCCAGGGGCATCTTCCGAGTGGTTCCACAGACAGTCCTGAATGAAGCCCAAGTCACAATCATTGTGGAGAA
CTCAGCTGCCATTGACTTTGAAAAGTCCAAAGTATTAACCTTCAAGCTCCTGGCTGTTGAAGTGAACACCCCAGAGAAGTTCAGTTCCACA
GCGGATGTTGTGATCCAGCTCCTGGACACCAATGACAATGTCCCCAAGTTCGACTCCCTCTACTACGTTGCCAGGATTCCTGAGAACGCCC
CAGGGGGCTCCAGCGTGGTGGCTGTCACAGCTGTGGATCCAGATACAGGACCCTGGGGCGAAGTGAAATATTCCACCTATGGGACTGGGGC
AGACCTCTTCCTGATCCACCCATCCACTGGGCTTATCTACACCCAGCCCTGGGCTAGCCTGGACGCTGAGGCCACTGCCAGGTACAACTTC
TATGTGAAGGCAGAGGACATGGAAGGCAAGTACAGCGTAGCTGAGGTGTTTATCACACTGCTGGATGTCAATGACCACCCCCCTCAGTTTG
GAAAGAGCGTTCAGAAGAAGACGATGGTGCTAGGGACCCCAGTGAAAATTGAGGCCATAGACGAGGATGCAGAGGAACCCAACAACCTGGT
GGACTATTCCATCACCCATGCAGAGCCCGCCAACGTGTTCGACATCAATTCCCACACGGGGGAGATCTGGCTCAAGAATTCCATCCGCTCC
CTGGATGCCCTGCACAACATCACACCTGGAAGGGACTGCCTATGGTCCCTAGAGGTGCAGGCCAAGGACCGGGGCTCCCCATCCTTCAGCA
CCACAGCCTTACTCAAGATTGACATCACAGATGCTGAGACCCTCTCCCGGAGCCCCATGGCTGCCTTCCTGATACAGACCAAGGACAACCC
CATGAAGGCCGTGGGTGTGCTGGCCGGCACCATGGCCACCGTCGTGGCCATCACTGTCCTCATCTCCACCGCCACCTTCTGGCGCAACAAG
AAGTCTAACAAGGTCCTGCCAATGCGGCGGGTGCTCCGCAAGCGGCCCAGCCCTGCGCCCCGCACCATCCGCATTGAGTGGCTCAAGTCCA
AGAGCACCAAAGCCGCTACCAAGTTCATGCTCAAAGAGAAACCTCCCAATGAGAACTGTAACAACAACAGCCCAGAAAGCTCTCTGCTCCC
GAGAGCTCCGGCTCTCCCTCCACCACCCAGCGTGGCGCCCAGCACTGGCGCAGCCCAGTGGACCGTGCCTACTGTCTCTGGCTCTCTCACT
CCGCAGCCGACCCAACCCCCGCCAAAACCCAAAACTATGGGAAGCCCCGTCCAGTCAACTCTGATCTCTGAGCTCAAGCAAAAGTTTGAGA
AGAAGAGTGTGCACAACAAGGCTTACTTCTAA
SEQ ID NO 6-Reference amino acid sequence for CDHR1
MRRCRWAALALGLLRLCLAQANFAPHFFDNGVGSTNGNMALFSLPEDTPVGSHVYTLNGTDPEGDPISYHISFDPSTRSVFSVDPTFGNIT
LVEELDREREDEIEAIISISDGLNLVAEKVVILVTDANDEAPRFIQEPYVALVPEDIPAGSIIFKVHAVDRDTGSGGSVTYFLQNLHSPFA
VDRHSGVLRLQAGATLDYERSRTHYITVVAKDGGGRLHGADVVFSATTTVTVNVEDVQDMAPVFVGTPYYGYVYEDTLPGSEVLKVVAMDG
DRGKPNRILYSLVNGNDGAFEINETSGAISITQSPAQLQREVYELHVQVTEMSPAGSPAAQATVPVTIRIVDLNNHPPTFYGESGPQNRFE
LSMNEHPPQGEILRGLKITVNDSDQGANAKENLQLVGPRGIFRVVPQTVLNEAQVTIIVENSAAIDFEKSKVLTFKLLAVEVNTPEKFSST
ADVVIQLLDTNDNVPKFDSLYYVARIPENAPGGSSVVAVTAVDPDTGPWGEVKYSTYGTGADLFLIHPSTGLIYTQPWASLDAEATARYNF
YVKAEDMEGKYSVAEVFITLLDVNDHPPQFGKSVQKKTMVLGTPVKIEAIDEDAEEPNNLVDYSITHAEPANVFDINSHTGEIWLKNSIRS
LDALHNITPGRDCLWSLEVQAKDRGSPSFSTTALLKIDITDAETLSRSPMAAFLIQTKDNPMKAVGVLAGTMATVVAITVLISTATFWRNK
KSNKVLPMRRVLRKRPSPAPRTIRIEWLKSKSTKAATKFMLKEKPPNENCNNNSPESSLLPRAPALPPPPSVAPSTGAAQWTVPTVSGSLT
PQPTQPPPKPKTMGSPVQSTLISELKQKFEKKSVHNKAYF
SEQ ID NO 7-WPRE
AATCAACCTCTGGATTACAAAATTTGTGAAAGATTGACTGGTATTCTTAACTATGTTGCTCCTTTTACGCTATGTGGATACGCTGCTTTAA
TGCCTTTGTATCATGCTATTGCTTCCCGTATGGCTTTCATTTTCTCCTCCTTGTATAAATCCTGGTTGCTGTCTCTTTATGAGGAGTTGTG
GCCCGTTGTCAGGCAACGTGGCGTGGTGTGCACTGTGTTTGCTGACGCAACCCCCACTGGTTGGGGCATTGCCACCACCTGTCAGCTCCTT
TCCGGGACTTTCGCTTTCCCCCTCCCTATTGCCACGGCGGAACTCATCGCCGCCTGCCTTGCCCGCTGCTGGACAGGGGCTCGGCTGTTGG
GCACTGACAATTCCGTGGTGTTGTCGGGGAAATCATCGTCCTTTCCTTGGCTGCTCGCCTGTGTTGCCACCTGGATTCTGCGCGGGACGTC
CTTCTGCTACGTCCCTTCGGCCCTCAATCCAGCGGACCTTCCTTCCCGCGGCCTGCTGCCGGCTCTGCGGCCTCTTCCGCGTCTTCGCCTT
CGCCCTCAGACGAGTCGGATCTCCCTTTGGGCCGCCTCCCCGC
SEQ ID NO 8-Bovine growth hormone polyadenylation tail
CGACTGTGCCTTCTAGTTGCCAGCCATCTGTTGTTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGGGGGTGGGGTGGGGCAGGACAGCAAGGGGGAGGAT
TGGGAAGACAATAGCAGGCATGCTGGGGATGCGGTGGGCTCTATGG
SEQ ID NO 9-DNA sequence for AAV2 ITR (3′-notated as reverse complement, 5′ to 3′)
TGCGCGCTCGCTCGCTCACTGAGGCCGCCCGGGCGTCGGGCGACCTTTGGTCGCCCGGCCTCAGTGAGCGAGCGAGCGCGCAGAGAGGGAG
TGGCCAACTCCATCACTAGGGGTTCCT
SEQ ID NO 10-Vector GRK1.CDHR1.pA (5′ITR to 3′ITR, inclusive)
TAGCTGCGCGCTCGCTCGCTCACTGAGGCCGCCCGGGCAAAGCCCGGGCGTCGGGCGACCTTTGGTCGCCCGGCCTCAGTGAGCGAGCGAG
CGCGCAGAGAGGGAGTGGCCAACTCCATCACTAGGGGTTCCTTGTAGTTAATGATTAACCCGCCATGCTACTTATCTACGTAGCCATGCTC
TAGGTACCGGGCCCCAGAAGCCTGGTGGTTGTTTGTCCTTCTCAGGGGAAAAGTGAGGCGGCCCCTTGGAGGAAGGGGCCGGGCAGAATGA
TCTAATCGGATTCCAAGCAGCTCAGGGGATTGTCTTTTTCTAGCACCTTCTTGCCACTCCTAAGCGTCCTCCGTGACCCCGGCTGGGATTT
AGCCTGGTGCTGTGTCAGCCCCGGGGTTAACGCCACCATGAGGCGCTGCCGGTGGGCCGCCCTGGCCCTGGGGCTGCTGCGCCTCTGCTTG
GCTCAGGCCAACTTCGCCCCGCACTTCTTCGACAACGGGGTCGGCAGCACCAACGGAAACATGGCTCTGTTCAGCCTCCCAGAGGACACCC
CTGTAGGCTCTCACGTATACACCCTGAATGGGACAGACCCTGAGGGAGACCCCATCTCCTACCACATCAGCTTTGACCCCAGCACTAGAAG
CGTCTTTTCTGTTGACCCCACTTTTGGAAACATCACCCTGGTTGAAGAGCTGGACAGAGAGAGGGAAGATGAGATTGAAGCCATCATCAGC
ATTTCTGATGGCCTGAATCTGGTGGCCGAAAAAGTCGTGATCCTGGTGACCGATGCCAATGATGAGGCGCCCAGGTTCATCCAGGAGCCTT
ATGTTGCCCTGGTTCCCGAGGACATACCTGCTGGGAGCATCATCTTTAAGGTCCATGCAGTGGACAGGGACACAGGCTCTGGAGGGAGTGT
CACCTACTTCCTGCAGAACCTGCACTCCCCATTTGCCGTGGACCGCCACAGCGGTGTGCTGCGCCTCCAGGCTGGGGCCACTCTGGACTAC
GAGAGGTCCCGGACCCACTACATCACCGTGGTCGCCAAGGATGGCGGTGGGAGGCTTCATGGGGCTGATGTGGTGTTCTCAGCCACCACCA
CGGTCACGGTCAATGTGGAGGATGTTCAGGACATGGCCCCTGTCTTCGTGGGCACACCCTACTATGGCTATGTGTACGAGGACACCCTTCC
GGGCTCGGAGGTACTGAAGGTGGTCGCCATGGATGGAGACCGGGGCAAACCCAATCGAATTCTCTACAGCCTTGTAAATGGGAACGATGGA
GCCTTTGAAATTAATGAGACATCTGGAGCCATCTCCATCACTCAGAGCCCGGCCCAGCTCCAGAGAGAGGTGTATGAGCTGCATGTACAGG
TGACTGAAATGAGCCCTGCGGGGAGCCCAGCTGCCCAGGCCACCGTCCCAGTCACCATCAGGATTGTGGACCTCAACAACCACCCGCCAAC
ATTCTATGGAGAGAGCGGACCCCAAAACAGGTTTGAGCTGTCCATGAATGAGCACCCACCCCAGGGAGAGATCCTGCGGGGCCTCAAGATC
ACCGTCAATGACTCCGACCAGGGAGCCAATGCCAAATTCAACTTGCAGCTGGTGGGACCCAGGGGCATCTTCCGAGTGGTTCCACAGACAG
TCCTGAATGAAGCCCAAGTCACAATCATTGTGGAGAACTCAGCTGCCATTGACTTTGAAAAGTCCAAAGTATTAACCTTCAAGCTCCTGGC
TGTTGAAGTGAACACCCCAGAGAAGTTCAGTTCCACAGCGGATGTTGTGATCCAGCTCCTGGACACCAATGACAATGTCCCCAAGTTCGAC
TCCCTCTACTACGTTGCCAGGATTCCTGAGAACGCCCCAGGGGGCTCCAGCGTGGTGGCTGTCACAGCTGTGGATCCAGATACAGGACCCT
GGGGCGAAGTGAAATATTCCACCTATGGGACTGGGGCAGACCTCTTCCTGATCCACCCATCCACTGGGCTTATCTACACCCAGCCCTGGGC
TAGCCTGGACGCTGAGGCCACTGCCAGGTACAACTTCTATGTGAAGGCAGAGGACATGGAAGGCAAGTACAGCGTAGCTGAGGTGTTTATC
ACACTGCTGGATGTCAATGACCACCCCCCTCAGTTTGGAAAGAGCGTTCAGAAGAAGACGATGGTGCTAGGGACCCCAGTGAAAATTGAGG
CCATAGACGAGGATGCAGAGGAACCCAACAACCTGGTGGACTATTCCATCACCCATGCAGAGCCCGCCAACGTGTTCGACATCAATTCCCA
CACGGGGGAGATCTGGCTCAAGAATTCCATCCGCTCCCTGGATGCCCTGCACAACATCACACCTGGAAGGGACTGCCTATGGTCCCTAGAG
GTGCAGGCCAAGGACCGGGGCTCCCCATCCTTCAGCACCACAGCCTTACTCAAGATTGACATCACAGATGCTGAGACCCTCTCCCGGAGCC
CCATGGCTGCCTTCCTGATACAGACCAAGGACAACCCCATGAAGGCCGTGGGTGTGCTGGCCGGCACCATGGCCACCGTCGTGGCCATCAC
TGTCCTCATCTCCACCGCCACCTTCTGGCGCAACAAGAAGTCTAACAAGGTCCTGCCAATGCGGCGGGTGCTCCGCAAGCGGCCCAGCCCT
GCGCCCCGCACCATCCGCATTGAGTGGCTCAAGTCCAAGAGCACCAAAGCCGCTACCAAGTTCATGCTCAAAGAGAAACCTCCCAATGAGA
ACTGTAACAACAACAGCCCAGAAAGCTCTCTGCTCCCGAGAGCTCCGGCTCTCCCTCCACCACCCAGCGTGGCGCCCAGCACTGGCGCAGC
CCAGTGGACCGTGCCTACTGTCTCTGGCTCTCTCACTCCGCAGCCGACCCAACCCCCGCCAAAACCCAAAACTATGGGAAGCCCCGTCCAG
TCAACTCTGATCTCTGAGCTCAAGCAAAAGTTTGAGAAGAAGAGTGTGCACAACAAGGCTTACTTCTAAAAGCTCTCGCTGATCAGCCTCG
ACTGTGCCTTCTAGTTGCCAGCCATCTGTTGTTTGCCCCTCCCCCGTGCCTTCCTTGACCCTGGAAGGTGCCACTCCCACTGTCCTTTCCT
AATAAAATGAGGAAATTGCATCGCATTGTCTGAGTAGGTGTCATTCTATTCTGGGGGGTGGGGTGGGGCAGGACAGCAAGGGGGAGGATTG
GGAAGACAATAGCAGGCATGCTGGGGATGCGGTGGGCTCTATGGCTTCTGAGGCGGAAAGAACCAGCTGGGGCTCGACTAGAGCATGGCTA
CGTAGATAAGTAGCATGGCGGGTTAATCATTAACTACAAGGAACCCCTAGTGATGGAGTTGGCCACTCCCTCTCTGCGCGCTCGCTCGCTC
ACTGAGGCCGGGCGACCAAAGGTCGCCCGACGCCCGGGCGGCCTCAGTGAGCGAGCGAGCGCGCA
SEQ ID NO 11-Vector GRK1.CDHR1.WPRE.pA (5′ITR to 3′ITR, inclusive)
TAGCTGCGCGCTCGCTCGCTCACTGAGGCCGCCCGGGCAAAGCCCGGGCGTCGGGCGACCTTTGGTCGCCCGGCCTCAGTGAGCGAGCGAG
CGCGCAGAGAGGGAGTGGCCAACTCCATCACTAGGGGTTCCTTGTAGTTAATGATTAACCCGCCATGCTACTTATCTACGTAGCCATGCTC
TAGGTACCGGGCCCCAGAAGCCTGGTGGTTGTTTGTCCTTCTCAGGGGAAAAGTGAGGCGGCCCCTTGGAGGAAGGGGCCGGGCAGAATGA
TCTAATCGGATTCCAAGCAGCTCAGGGGATTGTCTTTTTCTAGCACCTTCTTGCCACTCCTAAGCGTCCTCCGTGACCCCGGCTGGGATTT
AGCCTGGTGCTGTGTCAGCCCCGGGGTTATTGTTAACGCCACCATGAGGCGCTGCCGGTGGGCCGCCCTGGCCCTGGGGCTGCTGCGCCTC
TGCTTGGCTCAGGCCAACTTCGCCCCGCACTTCTTCGACAACGGGGTCGGCAGCACCAACGGAAACATGGCTCTGTTCAGCCTCCCAGAGG
ACACCCCTGTAGGCTCTCACGTATACACCCTGAATGGGACAGACCCTGAGGGAGACCCCATCTCCTACCACATCAGCTTTGACCCCAGCAC
TAGAAGCGTCTTTTCTGTTGACCCCACTTTTGGAAACATCACCCTGGTTGAAGAGCTGGACAGAGAGAGGGAAGATGAGATTGAAGCCATC
ATCAGCATTTCTGATGGCCTGAATCTGGTGGCCGAAAAAGTCGTGATCCTGGTGACCGATGCCAATGATGAGGCGCCCAGGTTCATCCAGG
AGCCTTATGTTGCCCTGGTTCCCGAGGACATACCTGCTGGGAGCATCATCTTTAAGGTCCATGCAGTGGACAGGGACACAGGCTCTGGAGG
GAGTGTCACCTACTTCCTGCAGAACCTGCACTCCCCATTTGCCGTGGACCGCCACAGCGGTGTGCTGCGCCTCCAGGCTGGGGCCACTCTG
GACTACGAGAGGTCCCGGACCCACTACATCACCGTGGTCGCCAAGGATGGCGGTGGGAGGCTTCATGGGGCTGATGTGGTGTTCTCAGCCA
CCACCACGGTCACGGTCAATGTGGAGGATGTTCAGGACATGGCCCCTGTCTTCGTGGGCACACCCTACTATGGCTATGTGTACGAGGACAC
CCTTCCGGGCTCGGAGGTACTGAAGGTGGTCGCCATGGATGGAGACCGGGGCAAACCCAATCGAATTCTCTACAGCCTTGTAAATGGGAAC
GATGGAGCCTTTGAAATTAATGAGACATCTGGAGCCATCTCCATCACTCAGAGCCCGGCCCAGCTCCAGAGAGAGGTGTATGAGCTGCATG
TACAGGTGACTGAAATGAGCCCTGCGGGGAGCCCAGCTGCCCAGGCCACCGTCCCAGTCACCATCAGGATTGTGGACCTCAACAACCACCC
GCCAACATTCTATGGAGAGAGCGGACCCCAAAACAGGTTTGAGCTGTCCATGAATGAGCACCCACCCCAGGGAGAGATCCTGCGGGGCCTC
AAGATCACCGTCAATGACTCCGACCAGGGAGCCAATGCCAAATTCAACTTGCAGCTGGTGGGACCCAGGGGCATCTTCCGAGTGGTTCCAC
AGACAGTCCTGAATGAAGCCCAAGTCACAATCATTGTGGAGAACTCAGCTGCCATTGACTTTGAAAAGTCCAAAGTATTAACCTTCAAGCT
CCTGGCTGTTGAAGTGAACACCCCAGAGAAGTTCAGTTCCACAGCGGATGTTGTGATCCAGCTCCTGGACACCAATGACAATGTCCCCAAG
TTCGACTCCCTCTACTACGTTGCCAGGATTCCTGAGAACGCCCCAGGGGGCTCCAGCGTGGTGGCTGTCACAGCTGTGGATCCAGATACAG
GACCCTGGGGCGAAGTGAAATATTCCACCTATGGGACTGGGGCAGACCTCTTCCTGATCCACCCATCCACTGGGCTTATCTACACCCAGCC
CTGGGCTAGCCTGGACGCTGAGGCCACTGCCAGGTACAACTTCTATGTGAAGGCAGAGGACATGGAAGGCAAGTACAGCGTAGCTGAGGTG
TTTATCACACTGCTGGATGTCAATGACCACCCCCCTCAGTTTGGAAAGAGCGTTCAGAAGAAGACGATGGTGCTAGGGACCCCAGTGAAAA
TTGAGGCCATAGACGAGGATGCAGAGGAACCCAACAACCTGGTGGACTATTCCATCACCCATGCAGAGCCCGCCAACGTGTTCGACATCAA
TTCCCACACGGGGGAGATCTGGCTCAAGAATTCCATCCGCTCCCTGGATGCCCTGCACAACATCACACCTGGAAGGGACTGCCTATGGTCC
CTAGAGGTGCAGGCCAAGGACCGGGGCTCCCCATCCTTCAGCACCACAGCCTTACTCAAGATTGACATCACAGATGCTGAGACCCTCTCCC
GGAGCCCCATGGCTGCCTTCCTGATACAGACCAAGGACAACCCCATGAAGGCCGTGGGTGTGCTGGCCGGCACCATGGCCACCGTCGTGGC
CATCACTGTCCTCATCTCCACCGCCACCTTCTGGCGCAACAAGAAGTCTAACAAGGTCCTGCCAATGCGGCGGGTGCTCCGCAAGCGGCCC
AGCCCTGCGCCCCGCACCATCCGCATTGAGTGGCTCAAGTCCAAGAGCACCAAAGCCGCTACCAAGTTCATGCTCAAAGAGAAACCTCCCA
ATGAGAACTGTAACAACAACAGCCCAGAAAGCTCTCTGCTCCCGAGAGCTCCGGCTCTCCCTCCACCACCCAGCGTGGCGCCCAGCACTGG
CGCAGCCCAGTGGACCGTGCCTACTGTCTCTGGCTCTCTCACTCCGCAGCCGACCCAACCCCCGCCAAAACCCAAAACTATGGGAAGCCCC
GTCCAGTCAACTCTGATCTCTGAGCTCAAGCAAAAGTTTGAGAAGAAGAGTGTGCACAACAAGGCTTACTTCTAAAAGCTTGGGAGCTTAT
CGATAATCAACCTCTGGATTACAAAATTTGTGAAAGATTGACTGGTATTCTTAACTATGTTGCTCCTTTTACGCTATGTGGATACGCTGCT
TTAATGCCTTTGTATCATGCTATTGCTTCCCGTATGGCTTTCATTTTCTCCTCCTTGTATAAATCCTGGTTGCTGTCTCTTTATGAGGAGT
TGTGGCCCGTTGTCAGGCAACGTGGCGTGGTGTGCACTGTGTTTGCTGACGCAACCCCCACTGGTTGGGGCATTGCCACCACCTGTCAGCT
CCTTTCCGGGACTTTCGCTTTCCCCCTCCCTATTGCCACGGCGGAACTCATCGCCGCCTGCCTTGCCCGCTGCTGGACAGGGGCTCGGCTG
TTGGGCACTGACAATTCCGTGGTGTTGTCGGGGAAATCATCGTCCTTTCCTTGGCTGCTCGCCTGTGTTGCCACCTGGATTCTGCGCGGGA
CGTCCTTCTGCTACGTCCCTTCGGCCCTCAATCCAGCGGACCTTCCTTCCCGCGGCCTGCTGCCGGCTCTGCGGCCTCTTCCGCGTCTTCG
CCTTCGCCCTCAGACGAGTCGGATCTCCCTTTGGGCCGCCTCCCCGCATCGATACCGTCGACTCGCTGATCAGCCTCGACTGTGCCTTCTA
GTTGCCAGCCATCTGTTGTTTGCCCCTCCCCCGTGCCTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGTGGGGTGGGGCAGGACAGCAAGGGGGAGGATTGGGAAGACAATAGC
AGGCATGCTGGGGATGCGGTGGGCTCTATGGCTTCTGAGGCGGAAAGAACCAGCTGGGGCTCGACTAGAGCATGGCTACGTAGATAAGTAG
CATGGCGGGTTAATCATTAACTACAAGGAACCCCTAGTGATGGAGTTGGCCACTCCCTCTCTGCGCGCTCGCTCGCTCACTGAGGCCGGGC
GACCAAAGGTCGCCCGACGCCCGGGCGGCCTCAGTGAGCGAGCGAGCGCGCA
SEQ ID NO 12-Vector GRK1.In.CDHR1.WPRE.pA (5′ITR to 3′ITR, inclusive)
TAGCTGCGCGCTCGCTCGCTCACTGAGGCCGCCCGGGCAAAGCCCGGGCGTCGGGCGACCTTTGGTCGCCCGGCCTCAGTGAGCGAGCGAG
CGCGCAGAGAGGGAGTGGCCAACTCCATCACTAGGGGTTCCTTGTAGTTAATGATTAACCCGCCATGCTACTTATCTACGTAGCCATGCTC
TAGGTACCGGGCCCCAGAAGCCTGGTGGTTGTTTGTCCTTCTCAGGGGAAAAGTGAGGCGGCCCCTTGGAGGAAGGGGCCGGGCAGAATGA
TCTAATCGGATTCCAAGCAGCTCAGGGGATTGTCTTTTTCTAGCACCTTCTTGCCACTCCTAAGCGTCCTCCGTGACCCCGGCTGGGATTT
AGCCTGGTGCTGTGTCAGCCCCGGGGTTATTGTTTCGCTGCGCGCTGCCTTCGCCCCGTGCCCCGCTCCGCCGCCGCCTCGCGCCGCCCGC
CCCGGCTCTGACTGACCGCGTTACTCCCACAGGTGAGCGGGCGGGACGGCCCTTCTCCTCCGGGCTGTAATTAGCGCTTGGTTTAATGACG
GCTTGTTTCTTTTCTGTGGCTGCGTGAAAGCCTTGAGGGGCTCCGGGAGGGCCCTTTGTGCGGGGGGAGCGGCTCGGGGCTGTCCGCGGGG
GGACGGCTGCCTTCGGGGGGGACGGGGCAGGGCGGGGTTCGGCTTCTGGCGTGTGACCGGCGGCTCTAGAGCCTCTGCTAACCATGTTCAT
GCCTTCTTCTTTTTCCTACAGCTCCTGGGCAACGTGCTGGTTATTGTGCTGTCTCATCATTTTGGCAAAGAATTGGATCCTAGCTTGATAT
CGAATTCCTGCAGCAACGCCACCAUGAGGCGCUGCCGGUGGGCCGCCCUGGCCCUGGGGCUGCUGCGCCUCUGCUUGGCUCAGGCCAACUU
CGCCCCGCACUUCUUCGACAACGGGGUCGGCAGCACCAACGGAAACAUGGCUCUGUUCAGCCUCCCAGAGGACACCCCUGUAGGCUCUCAC
GUAUACACCCUGAAUGGGACAGACCCUGAGGGAGACCCCAUCUCCUACCACAUCAGCUUUGACCCCAGCACUAGAAGCGUCUUUUCUGUUG
ACCCCACUUUUGGAAACAUCACCCUGGUUGAAGAGCUGGACAGAGAGAGGGAAGAUGAGAUUGAAGCCAUCAUCAGCAUUUCUGAUGGCCU
GAAUCUGGUGGCCGAAAAAGUCGUGAUCCUGGUGACCGAUGCCAAUGAUGAGGCGCCCAGGUUCAUCCAGGAGCCUUAUGUUGCCCUGGUU
CCCGAGGACAUACCUGCUGGGAGCAUCAUCUUUAAGGUCCAUGCAGUGGACAGGGACACAGGCUCUGGAGGGAGUGUCACCUACUUCCUGC
AGAACCUGCACUCCCCAUUUGCCGUGGACCGCCACAGCGGUGUGCUGCGCCUCCAGGCUGGGGCCACUCUGGACUACGAGAGGUCCCGGAC
CCACUACAUCACCGUGGUCGCCAAGGAUGGCGGUGGGAGGCUUCAUGGGGCUGAUGUGGUGUUCUCAGCCACCACCACGGUCACGGUCAAU
GUGGAGGAUGUUCAGGACAUGGCCCCUGUCUUCGUGGGCACACCCUACUAUGGCUAUGUGUACGAGGACACCCUUCCGGGCUCGGAGGUAC
UGAAGGUGGUCGCCAUGGAUGGAGACCGGGGCAAACCCAAUCGAAUUCUCUACAGCCUUGUAAAUGGGAACGAUGGAGCCUUUGAAAUUAA
UGAGACAUCUGGAGCCAUCUCCAUCACUCAGAGCCCGGCCCAGCUCCAGAGAGAGGUGUAUGAGCUGCAUGUACAGGUGACUGAAAUGAGC
CCUGCGGGGAGCCCAGCUGCCCAGGCCACCGUCCCAGUCACCAUCAGGAUUGUGGACCUCAACAACCACCCGCCAACAUUCUAUGGAGAGA
GCGGACCCCAAAACAGGUUUGAGCUGUCCAUGAAUGAGCACCCACCCCAGGGAGAGAUCCUGCGGGGCCUCAAGAUCACCGUCAAUGACUC
CGACCAGGGAGCCAAUGCCAAAUUCAACUUGCAGCUGGUGGGACCCAGGGGCAUCUUCCGAGUGGUUCCACAGACAGUCCUGAAUGAAGCC
CAAGUCACAAUCAUUGUGGAGAACUCAGCUGCCAUUGACUUUGAAAAGUCCAAAGUAUUAACCUUCAAGCUCCUGGCUGUUGAAGUGAACA
CCCCAGAGAAGUUCAGUUCCACAGCGGAUGUUGUGAUCCAGCUCCUGGACACCAAUGACAAUGUCCCCAAGUUCGACUCCCUCUACUACGU
UGCCAGGAUUCCUGAGAACGCCCCAGGGGGCUCCAGCGUGGUGGCUGUCACAGCUGUGGAUCCAGAUACAGGACCCUGGGGCGAAGUGAAA
UAUUCCACCUAUGGGACUGGGGCAGACCUCUUCCUGAUCCACCCAUCCACUGGGCUUAUCUACACCCAGCCCUGGGCUAGCCUGGACGCUG
AGGCCACUGCCAGGUACAACUUCUAUGUGAAGGCAGAGGACAUGGAAGGCAAGUACAGCGUAGCUGAGGUGUUUAUCACACUGCUGGAUGU
CAAUGACCACCCCCCUCAGUUUGGAAAGAGCGUUCAGAAGAAGACGAUGGUGCUAGGGACCCCAGUGAAAAUUGAGGCCAUAGACGAGGAU
GCAGAGGAACCCAACAACCUGGUGGACUAUUCCAUCACCCAUGCAGAGCCCGCCAACGUGUUCGACAUCAAUUCCCACACGGGGGAGAUCU
GGCUCAAGAAUUCCAUCCGCUCCCUGGAUGCCCUGCACAACAUCACACCUGGAAGGGACUGCCUAUGGUCCCUAGAGGUGCAGGCCAAGGA
CCGGGGCUCCCCAUCCUUCAGCACCACAGCCUUACUCAAGAUUGACAUCACAGAUGCUGAGACCCUCUCCCGGAGCCCCAUGGCUGCCUUC
CUGAUACAGACCAAGGACAACCCCAUGAAGGCCGUGGGUGUGCUGGCCGGCACCAUGGCCACCGUCGUGGCCAUCACUGUCCUCAUCUCCA
CCGCCACCUUCUGGCGCAACAAGAAGUCUAACAAGGUCCUGCCAAUGCGGCGGGUGCUCCGCAAGCGGCCCAGCCCUGCGCCCCGCACCAU
CCGCAUUGAGUGGCUCAAGUCCAAGAGCACCAAAGCCGCUACCAAGUUCAUGCUCAAAGAGAAACCUCCCAAUGAGAACUGUAACAACAAC
AGCCCAGAAAGCUCUCUGCUCCCGAGAGCUCCGGCUCUCCCUCCACCACCCAGCGUGGCGCCCAGCACUGGCGCAGCCCAGUGGACCGUGC
CUACUGUCUCUGGCUCUCUCACUCCGCAGCCGACCCAACCCCCGCCAAAACCCAAAACUAUGGGAAGCCCCGUCCAGUCAACUCUGAUCUC
UGAGCUCAAGCAAAAGUUUGAGAAGAAGAGUGUGCACAACAAGGCUUACUUCUAGAAGCTTGGGAGCTTATCGATAATCAACCTCTGGATT
ACAAAATTTGTGAAAGATTGACTGGTATTCTTAACTATGTTGCTCCTTTTACGCTATGTGGATACGCTGCTTTAATGCCTTTGTATCATGC
TATTGCTTCCCGTATGGCTTTCATTTTCTCCTCCTTGTATAAATCCTGGTTGCTGTCTCTTTATGAGGAGTTGTGGCCCGTTGTCAGGCAA
CGTGGCGTGGTGTGCACTGTGTTTGCTGACGCAACCCCCACTGGTTGGGGCATTGCCACCACCTGTCAGCTCCTTTCCGGGACTTTCGCTT
TCCCCCTCCCTATTGCCACGGCGGAACTCATCGCCGCCTGCCTTGCCCGCTGCTGGACAGGGGCTCGGCTGTTGGGCACTGACAATTCCGT
GGTGTTGTCGGGGAAATCATCGTCCTTTCCTTGGCTGCTCGCCTGTGTTGCCACCTGGATTCTGCGCGGGACGTCCTTCTGCTACGTCCCT
TCGGCCCTCAATCCAGCGGACCTTCCTTCCCGCGGCCTGCTGCCGGCTCTGCGGCCTCTTCCGCGTCTTCGCCTTCGCCCTCAGACGAGTC
GGATCTCCCTTTGGGCCGCCTCCCCGCATCGATACCGTCGACTCGCTGATCAGCCTCGACTGTGCCTTCTAGTTGCCAGCCATCTGTTGTT
TGCCCCTCCCCCGTGCCTTCCTTGACCCTGGAAGGTGCCACTCCCACTGTCCTTTCCTAATAAAATGAGGAAATTGCATCGCATTGTCTGA
GTAGGTGTCATTCTATTCTGGGGGGTGGGGTGGGGCAGGACAGCAAGGGGGAGGATTGGGAAGACAATAGCAGGCATGCTGGGGATGCGGT
GGGCTCTATGGCTTCTGAGGCGGAAAGAACCAGCTGGGGCTCGACTAGAGCATGGCTACGTAGATAAGTAGCATGGCGGGTTAATCATTAA
CTACAAGGAACCCCTAGTGATGGAGTTGGCCACTCCCTCTCTGCGCGCTCGCTCGCTCACTGAGGCCGGGCGACCAAAGGTCGCCCGACGC
CCGGGCGGCCTCAGTGAGCGAGCGAGCGCGCA
SEQ ID NO 13-CDHR1 extracellular cadherin repeat domain 1
FSLPEDTPVGSHVYTLNGTDPEGDPISYHISFDPSTRSVFSVDPTFGNITLVEELDREREDEIEAIISISDGLNLVAEKVVILVTDAND
SEQ ID NO 14-CDHR1 extracellular cadherin repeat domain 2
PYVALVPEDIPAGSIIFKVHAVDRDTGSGGSVTYFLQNLHSPFAVDRHSGVLRLQAGATLDYERSRTHYITVVAKDGGGRLHGADVVFSAT
TTVTVNVEDVQD
SEQ ID NO 15-CDHR1 extracellular cadherin repeat domain 3
PYYGYVYEDTLPGSEVLKVVAMDGDRGKPNRILYSLVNGNDGAFEINETSGAISITQSPAQLQREVYELHVQVTEMSPAGSPAAQATVPVT
IRIVDLNNH
SEQ ID NO 16-CDHR1 extracellular cadherin repeat domain 4
FELSMNEHPPQGEILRGLKITVNDSDQGANAKFNLQLVGPRGIFRVVPQTVLNEAQVTIIVENSAAIDFEKSKVLTFKLLAVEVNTPEKES
STADVVIQLLDTNDN
SEQ ID NO 17-CDHR1 extracellular cadherin repeat domain 5
YVARIPENAPGGSSVVAVTAVDPDTGPWGEVKYSTYGTGADLFLIHPSTGLIYTQPWASLDAEATARYNFYVKAEDMEGKYSVAEVFITLL
DVNDH
SEQ ID NO 18-CDHR1 extracellular cadherin repeat domain 6
GTPVKIEAIDEDAEEPNNLVDYSITHAEPANVEDINSHTGEIWLKNSIRSLDALHNITPGRDCLWSLEVQAKDRGSPSFSTTALLKIDITD
A

1. A method of treating, preventing or reversing retinal degeneration in a subject in need thereof, the method comprising administering to the subject a vector that expresses a Cadherin-related family member 1 (CDHR1) polypeptide.

2. A gene therapy vector that expresses a CDHR1 polypeptide.

3. The method according to claim 1 or the gene therapy vector according to claim 2, wherein the vector is a viral vector.

4. The method or gene therapy vector according to claim 3, wherein the viral vector is an adeno-associated virus (AAV) vector.

5. The method or gene therapy vector according to claim 4, wherein the vector comprises an AAV genome or a derivative thereof.

6. The method or gene therapy vector according to claim 5, wherein the AAV genome is AAV serotype 2 (AAV2) and/or wherein the capsid is serotype 8 (AAV8).

7. The method or gene therapy vector according to claim 5, wherein the capsid comprises wildtype AAV8 capsid having the amino acid sequence of SEQ ID NO: 13.

8. The method or the gene therapy vector according to any one of the preceding claims, wherein the vector comprises, in a 5′ to 3′ direction:
(a) a 5′ inverted terminal repeat sequence (5′ITR);
(b) a promoter sequence, wherein the promoter is operably linked to a polynucleotide sequence encoding the CDHR1 polypeptide;
(c) a translation initiation sequence;
(d) optionally a chicken beta-actin promoter exon-intron-exon sequence (Ex/In/Ex);
(e) the polynucleotide sequence encoding the CDHR1 polypeptide;
(f) optionally a woodchuck hepatitis post-transcriptional regulatory element (WPRE) having the sequence of SEQ ID NO: 7;
(g) a polyadenylation tail sequence; and
(h) a 3′ inverted terminal repeat sequence (3′ITR).

9. The method or the gene therapy vector according to any one of the preceding claims, wherein
(i) the 5′ITR has the sequence of SEQ ID NO: 1;
(ii) expression of the CDHR2 polypeptide is controlled by a human rhodopsin kinase promoter (GRK1);
(iii) the translation initiation sequence comprises the Kozak consensus sequence GCCACC;
(iv) the Ex/In/Ex comprises the sequence of SEQ ID NO: 4;
(v) the CDHR1 polypeptide comprises the amino acid sequence of SEQ ID NO: 6, or a variant thereof having at least 80% sequence identity to SEQ ID NO: 6;
(vi) the WPRE comprises the sequence of SEQ ID NO: 7;
(vii) the polyadenylation tail sequence comprises a bovine growth hormone polyadenylation tail sequence of SEQ ID NO: 8; and/or
(viii) the 5′ITR has the sequence of SEQ ID NO: 9.

10. The method or the gene therapy vector according to any one of the preceding claims, wherein the vector comprises a sequence selected from SEQ ID NO: 10, a sequence having at least 80% sequence identity to SEQ ID NO: 10, SEQ ID NO: 12, a sequence having at least 80% sequence identity to SEQ ID NO: 11, SEQ ID NO: 12, and a sequence having at least 80% sequence identity to SEQ ID NO: 12.

11. A host cell that produces the gene therapy vector of any one of claims 2 to 10.

12. A pharmaceutical composition comprising the gene therapy vector according to any one claims 2 to 10, and optionally at least one pharmaceutically acceptable diluent, carrier, or preservative.

13. The method, gene therapy vector or pharmaceutical composition according to any one of the preceding claims, wherein the gene therapy vector or pharmaceutical composition is administered or for administration by sub-retinal injection.

14. The gene therapy vector according to any one of claims 2 to 10 or the pharmaceutical composition of claim 12, for use in a method of treating, preventing or reversing CDHR1-associated retinal degeneration.

15. Use of a gene therapy vector according to any one of claims 2 to 10 in the manufacture of a medicament for of treating, preventing or reversing CDHR1-associated retinal degeneration in a subject.
